SEQUENCE LISTING (98 sequences)

SEQ ID NO: 1 - DNA, 4531 bp, Parachlorella sp.
  misc_feature 1..4531: note = SGI1 gene
  source 1..4531: mol_type = unassigned DNA
atgtctggttcagctggatcgggccaggctactctcagacatgacggtggctctgctggcggcagtgggcctgtctcagacggtttttcaccggccggcctgaaggtaaagtagaaagacactcatacacatcttggttcggcgttgaaagtaggtcattaacatactctataaccaatatttgtaggttctggtcgtggacgacgacctcatgtgccttaaggtggtgtcagccatgttgaagaggtgcagctatcaaggtgaggtctttactggtgtctgttattgctgtaacatcatttcgctgttgcacaatttaaacatttgtaatttactgttgttattgcagtggccacttgtagcagtggcagcgaggcactgacacttctacgtgaacgcaacgaggacggatcctccgaccagttcgacctcgtactgtcagatgtttacatgccgggtatgtcgtattcctttgtaaactttacaatatgcgtctagtttgacgcgtacactttgtacactttgcaaaaacgcaccctgcgaggtctgccatttggtcactacaacttggccaccttggttgcaagtttgcaagttcgctctacgtcaacgctgcaaaatgaaccaattgttttgcactgaccctgccaaccttcatttgtggctgcagacatggacggtttcaagctgcttgaacacatcggtctagagttggagcttcccgttatcagtaagttgatcgagccgagtccagagcgaagcctgcttctatactattagcagctgtcttttgatatttgacagcttgacttgatatggtcacagagcatacttgcaaccaggttacctgttgaactagcaactgtgcccaagcatctcttcaagcacctccgtcagtccatagggtactgttgatttgtactctgcaatactgcactgtaatgcgctgtgaatcactgcccttcacctctagatggtgcttccctggagccctcccccacctccgcctcaagcccctcacatgcctctcccccccctgcagtgatgtcatccaacggggacacgaatgtcgtgctgcggggggtcacccacggggctgtggactttctgatcaagcccgttcgaattgaggagctgcggaacgtgtggcagcacgtggtgcgtcgtcgttccatggcgctggccaggacgccagacgaggggggacactcggacgaggactctcaggtgcccttggcagcttctgggcggcttgctgtgtcggatgccacttggactggggatgcacgaggggtggggggacaatgggagatgggccatagtaggccagagttgatggcagtggtggtgggggggagtaggcgggagagaagcagccatcctggtgttggttttgatgattgagtgcatggggatgatgcacaggtgagctgactggatgccttgtcttgctgtgctgcgctgcagcggcacagtgtgaaacgcaaggagtcggagcagagcccgctgcagctcagcacagagcagggcgggaacaagaagccaagagtggtgtggtcggtggagatgcaccaacaggtgtgcttgcgggcgggtgtatacgggggaggggggccagctgctggctgacctggcgtgcgcggtgcattgcacttggcgatgaggggcgtgcttcagtatgtagctgggacgcaattggttgtgctgtgtgaccagtgcacaaaatacatccctgaattccagtgggttgaacagagttgtcctggaggtgggaagcaaacgcgcacgtggtagaggggagcagggtgcagaacagccgcagcaggggtgttgcgcagtgtgcaggtatcctgcctccatgccccgggccatgggcatactacgctggtaccgtcaggatgggcgttgagcctggcttggggggcagggggcgagcgaatgcggaatgggagcggcaggtgctgggagggtggctgactggcttgcaggagcgcaagtcctgtcgggggcgtcgtcctgttccctcctgcccgcttcacccacgttcactctcatgcctccacactcctgctgctgacacacctgtcgccacctccgctgcagtttgtgaacgcggtcaactccctgggcattgacaaggcggtgcccaagcggattctggacctgatgaacgtggaggggctgacgcgcgagaacgtggccagccatctgcaggtgcctgccatgacccctcccaccagggacctggtgttttgacaccctggaactcctctttgacggagcctccagttcaattccagcaatcgaattgaatcaaaaagcatgtgcacccacgtgctgtttgaatgtcccatgtggtaggaaacacaactgcccccttgccatttgctggagggtgcccgctgcgccatgcccgagtgcgctgtgctcagcgttgtgctgcgccccccgctgactgaagctgacagcgtgcggctgaggagggtactgggggagggggggtgggaggcggccgctggcggcggaagggagggtgtgcacgcatggacacagggcctttccgccctgcacggcctctactgcaccctgccacgtgatgtatcgacatggtgggccatgctgtgctgtgccgctgcagaagtaccgcctgtacctgaagcgggtggagggagtgcaatcgggtgcggcagcctccaagcagcaccagcacccgcagtatcaccagcagcagcagcagcagcaagcgcaacctcgtgcagctgtctcccctgcagcagcttcctttggtgccctttccttgggagccccgcagcaggcgcagcagggcatgccgcagctggggatgcctgtgcaggtgaagactgcccccccccccctccccctttccatcttccctccatcagcctgctgttccttacccttgtcaacccgtctctcctttttcgcaagcagcgcaccaccccccatgcacgccttgcctggcactgttgtcagctgcccccctagaaatacacaaggtgtgggtgcaactggtgggaccccctcccccccccccctggggctgcagggtctccctccaaacttggcagccatgggatcccagccgccgcacatccccttccagcaggccctggccatgcaggcggcggctgcggcggctgcagccagcggcgcgctccccgggagtctgcccccctacatgccacccccggggatgatgccccccggcatgccggggggggtccccggtatgggaggggtggtggggcatcctcaggtacgggcagcacatgagtgggcaggggtattggagaggggaagggcagggaggttgcatgtgaggggctgcatggcaaagaggctgcagcgcaggtgttgcttgcagcacttcccctcggtggcgcttgcatcaaattttgaatcctcccccgatgggcacgcccgtgtgtgggggggggtgggatgggggatgggggtggttttgtggcatgtcgggcgctttcatctacccgggcccctgcccctgcctgtacgcgtgcgcatgtgtgcagatgcccgccccagggatggactttgcgggtttcaacgggtatggcaacgctgcgggggggctgatgtttggcgggcagcagcaggcgcagcacgcgcagcagcacgcgtcagcgcaagcgggctcgctggcgcagcagcaggcgcagcaagtatccatgggcttgggccttatgccccccccgttggggttcccgcccacctcgctcgccgcgccagccccgcgctccgcagcaactgagcccgccgcagccccactccccctgacgtcctcgccgccagctgcttcagcaggcggcagcggcggcccagcagcagctgctccgcagcacagcagcggcgccgcagcagcccaagccccccatcaccacccacagtgctcggagcagggagcgggggggctcccgcccccgctgcccgcgtccagcgccccgcagtcctatcccctccctcccccctcctcgcaggccgctttgcatgacccggacgaacactaccccccaggctcggcagaggtgagcacgtccccccgccccctccccccccccccccccccttcccttcaccctggcttggcgtgcaatgaaaccctaaataaccctaaaacctcattatcagttgcaaattggacccgtgaagcgggcgggggcaactgcgctctgctggtgtcagcgctgtctctgccggttcctgcccagcgtgcgcctgcatgcaaggggggatggggggggggaggcatttaacaataggccagtcatctccaatccaccgtcaatttcagccccctccccccccctccctcatccccttgcagatgcaccaccagcacctcccagggctgtgtggctttaacccggacgacctgctgggggggcagctgggggacatggggttcctgggggagctggggggggcggtgggaggaaagcacgaacaggacgacttcctggacctgctgctgaagggggaggaggagctgtga

SEQ ID NO: 2 - DNA, 1860 bp, Parachlorella sp.
  misc_feature 1..1860: note = SGI1 coding sequence
  source 1..1860: mol_type = unassigned DNA
atgtctggttcagctggatcgggccaggctactctcagacatgacggtggctctgctggcggcagtgggcctgtctcagacggtttttcaccggccggcctgaaggttctggtcgtggacgacgacctcatgtgccttaaggtggtgtcagccatgttgaagaggtgcagctatcaagtggccacttgtagcagtggcagcgaggcactgacacttctacgtgaacgcaacgaggacggatcctccgaccagttcgacctcgtactgtcagatgtttacatgccggacatggacggtttcaagctgcttgaacacatcggtctagagttggagcttcccgttatcatgatgtcatccaacggggacacgaatgtcgtgctgcggggggtcacccacggggctgtggactttctgatcaagcccgttcgaattgaggagctgcggaacgtgtggcagcacgtggtgcgtcgtcgttccatggcgctggccaggacgccagacgaggggggacactcggacgaggactctcagcggcacagtgtgaaacgcaaggagtcggagcagagcccgctgcagctcagcacagagcagggcgggaacaagaagccaagagtggtgtggtcggtggagatgcaccaacagtttgtgaacgcggtcaactccctgggcattgacaaggcggtgcccaagcggattctggacctgatgaacgtggaggggctgacgcgcgagaacgtggccagccatctgcagaagtaccgcctgtacctgaagcgggtggagggagtgcaatcgggtgcggcagcctccaagcagcaccagcacccgcagtatcaccagcagcagcagcagcagcaagcgcaacctcgtgcagctgtctcccctgcagcagcttcctttggtgccctttccttgggagccccgcagcaggcgcagcagggcatgccgcagctggggatgcctgtgcagggtctccctccaaacttggcagccatgggatcccagccgccgcacatccccttccagcaggccctggccatgcaggcggcggctgcggcggctgcagccagcggcgcgctccccgggagtctgcccccctacatgccacccccggggatgatgccccccggcatgccggggggggtccccggtatgggaggggtggtggggcatcctcagatgcccgccccagggatggactttgcgggtttcaacgggtatggcaacgctgcgggggggctgatgtttggcgggcagcagcaggcgcagcacgcgcagcagcacgcgtcagcgcaagcgggctcgctggcgcagcagcaggcgcagcaagtatccatgggcttgggccttatgccccccccgttggggttcccgcccacctcgctcgccgcgccagccccgcgctccgcagcaactgagcccgccgcagccccactccccctgacgtcctcgccgccagctgcttcagcaggcggcagcggcggcccagcagcagctgctccgcagcacagcagcggcgccgcagcagcccaagccccccatcaccacccacagtgctcggagcagggagcgggggggctcccgcccccgctgcccgcgtccagcgccccgcagtcctatcccctccctcccccctcctcgcaggccgctttgcatgacccggacgaacactaccccccaggctcggcagagatgcaccaccagcacctcccagggctgtgtggctttaacccggacgacctgctgggggggcagctgggggacatggggttcctgggggagctggggggggcggtgggaggaaagcacgaacaggacgacttcctggacctgctgctgaagggggaggaggagctgtga

SEQ ID NO: 3 - protein, 619 aa, Parachlorella sp.
  REGION 1..619: note = misc_feature - SGI1 polypeptide
MSGSAGSGQATLRHDGGSAGGSGPVSDGFSPAGLKVLVVDDDLMCLKVVSAMLKRCSYQVATCSSGSEALTLLRERNEDGSSDQFDLVLSDVYMPDMDGFKLLEHIGLELELPVIMMSSNGDTNVVLRGVTHGAVDFLIKPVRIEELRNVWQHVVRRRSMALARTPDEGGHSDEDSQRHSVKRKESEQSPLQLSTEQGGNKKPRVVWSVEMHQQFVNAVNSLGIDKAVPKRILDLMNVEGLTRENVASHLQKYRLYLKRVEGVQSGAAASKQHQHPQYHQQQQQQQAQPRAAVSPAAASFGALSLGAPQQAQQGMPQLGMPVQGLPPNLAAMGSQPPHIPFQQALAMQAAAAAAAASGALPGSLPPYMPPPGMMPPGMPGGVPGMGGVVGHPQMPAPGMDFAGFNGYGNAAGGLMFGGQQQAQHAQQHASAQAGSLAQQQAQQVSMGLGLMPPPLGFPPTSLAAPAPRSAATEPAAAPLPLTSSPPAASAGGSGGPAAAAPQHSSGAAAAQAPHHHPQCSEQGAGGLPPPLPASSAPQSYPLPPPSSQAALHDPDEHYPPGSAEMHHQHLPGLCGFNPDDLLGGQLGDMGFLGELGGAVGGKHEQDDFLDLLLKGEEEL

SEQ ID NO: 4 - DNA, 23 bp, Parachlorella sp.
  misc_feature 1..23: note = Target sequence in SGI1 gene of Parachlorella strain WT-1185, including PAM
  source 1..23: mol_type = unassigned DNA
acaccaccttaaggcacatgagg

SEQ ID NO: 5 - RNA, 105 nt, synthetic construct
  misc_feature 1..105: note = Synthetic
  misc_feature 1..105: note = Guide RNA for targeting Parachlorella SGI1 gene
  source 1..105: mol_type = other RNA
ggacaccaccttaaggcacatggttttagagctagaaatagcaagttaaaataaggctagtccgttatcaacttgaaaaagtggcaccgagtcggtgcttttttt

SEQ ID NO: 6 - DNA, 2667 bp, synthetic construct
  misc_feature 1..2667: note = Synthetic
  misc_feature 1..2667: note = bleomycin resistance "BleR" gene, codon optimized for Parachlorella with Parachlorella introns
  source 1..2667: mol_type = other DNA
atggccaaactgacatccgctgttcctgtgttgacagcaagagatgttgcaggtgcagtggagttttgtgagttctgagaagctgattgttgtttaacttctttgaaagctttatcgaagattctgcaagcgatgaacattgcttgtcaagaccgagagctgcatgcccacttgacatccagctttgaacggctcttcatgtttgatttgtttctgattgtagggacagatagactggggtttagcagggactttgtggaggacgattttgcaggagtggtgagggatgatgtgacactgtttatctcagcagtgcaggatcaagtgagtgcagcgtcagctgtggcagttgttggctttcgtctcagtcagtagtttgctgggattgattatggagggcacagttgcaattttgagttgcacgttgcgacaagcgtgttgacaaagcgtggtcaagccggccagtcttgccggtggcgggtggcttggtctaacttccgctctacagcaatcgttttgttcatggttacggggctggcgtgccagaaagtcctggtcagccaccctcgcttcaaagccgtagcccaacaactttgcgaatatgttcgatttgcaggtggtgcccgataatacactggcatgggtttgggtgagaggtacagctctgcgtgcaacaggttgcaagatgcagcgcaggtcttccctggtcaaacgatgtatgcagagttgagaggcacttgagctgggtgaatggcgtgggctcgtaggtagtgtgcagggcaggaagggcagccaattttggagttgtggtccggtgtcgttgcttcgagccttattaggactcttgctcatcaaagcgttagttgtgaataagttgatctgaaaggatgttatgtacagcaagcagcagcagttaagagtctggggagtagctgcacagggcgaggtgtcaagatgggaagggtcctgcctccttatgtgtttttccctgtaggggaggaagcctcttatgggcaatggttgggcatattttccagccagcccttctttctataggggccagggtgggcccagctcgtcttggcttccaccaccaggagagtgagggcattgaagggccataaatagtcctcccatctacgtgcaccagagggtgtcgtctaggctgtgcatgccacgaggggaaggagccaagaatgagtgtatgggttgttttcatgtttaggctgggataaaactgttttcaattgcgcctgccgggtgaaaaccacagcagcatcagcaagcttggagaaggccagcccgcccagcacaggctcacgttcccactcaggcggtcagtcgggcgggggtgtgagtcaggcaggcgagggtgtctgtgcctgacatcagcacctctgcttagccactgcagcccctggagcagggtagggcgtcatttgcagcaatcacctgctgcctcacacgtcgcagcttggaatttcaacgaccatcagcgctggggttgttgagggatcatagcagattttggtgcagcctggttgtcatgctctttgtggaatggcctctatgttcgagcaattcgttggatgttgaggtgcttggggacagagagtcgaatgatgggccagggtcaaacatgcgagcgtttggctgagtcagcggtttttgctggtcactttttcttttgtttcttatttaggtttgatggatgtgttttgtgctgctgccctgaagctgcagcagcgtgtctgccctgcgctactgcgggcaccaaggctatgtgctggtgcactcggctgcgctgcacctgtgcacctcgcactccgtccagcctccatgcagcacacgtactcacggtgtcctcctgacctgtcgtacgctattccaaacttgctcttttgctgccgctgctctcgtacacaattgctgttgattatcgatatctaatcgagcgcctgctgactgaactccgcaggtttggatgaactgtatgcagagtggtctgaagtggtgagcaccaactttaggtgggtgggctctgaaggaggaggagggagcgggtgattaaacagggcctgcatgaagaggagcaggggctgcatggacagcagggggaaggtgcagaagggagggtcaagcggggttcaggtggctgtgggtttctgcacgagcagtgaaagaagctgtatccttccacctgctttcactggcgaaaggttgaaaacaggatgtcgcagctggaaagatgttgcgctgtcaagtgcaagccatggttgagggtatgcctgtgtgcatgtgcttcttaaagttactcctgttctatggttctgggtgcttgttgtttgtggtgcagggatgcaagcggacctgcaatgacagagattggagaacaaccttggggaagggagtttgcattgagagatcctgcaggtgagggggcatgtaagcaatggcaggcaattcaagaacgaatcattgctgcaaatgctgggatggtatgcagctgaggtatctattgccttgtattttgtctcgcattgcatcggtggtgcgttctgtggcctgaggcacagttcttgctgtttgataagggttcgactgagttgtcgtgtgtgctgtgctgcaggcaattgcgtgcactttgttgcagaagaacaggactga

SEQ ID NO: 7 - DNA, 530 bp, Parachlorella sp.
  misc_feature 1..530: note = RPS4 promoter
  source 1..530: mol_type = unassigned DNA
ccaccatgggggaggtttgaagtgtgcgcctgatataatcatacacctaaaagcaccacttgctgattgtgaagggactatgtcgtttatgacgggacgttacgctggccgatggtttgaatttggacgctgtggtagaatgttatatggacgtaaaggttggcatattgaaaatcgtcttcgcaggcaaacttctagacgtgtgacccaccggtaaaacgacaagcgtggcgcgtcgattgcgctttgaacgtcgtttgttggactccagatgaacctcaaaatcaaagcggtgattgacgaaaatcaaatgacagcccgcaaaatttcatcagccttcggatcggattctcagaatctgattgtccctgctggctacatttatgaaatttcgtacattttggcagaaatgtcccaataccatagcactgccgcctgagctcacccgagcaatgcatactgggtacctcgcccatctcgccctctttccaagcccagtgctgttgtaatagccaaagggctcagtaaca

SEQ ID NO: 8 - DNA, 546 bp, Parachlorella sp.
  misc_feature 1..546: note = RPS4 terminator
  source 1..546: mol_type = unassigned DNA
gcatagcatcagcctgtggcagggttgtggtagggctgagtggcagggttaaaggggttgcctaccccacccctactctcatgacaccagcaacagcagcagctcatgcagtactcaaatcactgatgtcaatggtgtgacacatttggttaaggctgctttttaaagtgctgctttgggggcagtgactgtgcagagcttggagcgtatccccatgtaatcagaaccgacgagagttcggggcaacctttcatcttcacattttttgtgatcagctacagagtctgaaatcaaatagaggctgccatctaaacgcaggagtcacaacgaaggcgaaaactccaattgctgtactcaatgcactaagtgattgttcaatggataaatacactatgctcaattcatgccagcagagctgctccttccagccagctacaatggctttttccacgccttttgaagtatgaatgttcagcttgctgtgcttgatgcatcaccataaacacaattctacaacatttcatgccaacaacagtacgggctttc

SEQ ID NO: 9 - protein, 302 aa, unidentified
  REGION 1..302: note = misc_feature - SGI1 polypeptide, NCBI Accession XP_005652114
  source 1..302: note = Coccomyxa subellipsoidea
MGLKARAASVSVHSSANNTASPLSSGRRGFPHSGEMSGEDLARSDSWEMFPAGLKVLVVDDDPLCLKVVEHMLRRCNYQVTTCPNGKAALEKLRDRSVHFDLVLSDVYMPDMDGFKLLEHIGLELDLPVIMMSSNGETNVVLRGVTHGAVDFLIKPVRVEELRNVWQHVVRRKRDQAVSQARDSRDISDEEGTDDGKPRDKKRKEVILVLWWDMQRRDSDDGVSAKKARVVWSVEMHQQFVQAVNQLGIDKAVPKRILDLMNVDGLTRENVASHLQVPHLSIFSPLFAELMSTLPRRCFYDF

SEQ ID NO: 10 - protein, 270 aa, unidentified
  REGION 1..270: note = misc_feature - SGI1 polypeptide, NCBI Accession XP_001415508
  source 1..270: note = Ostreococcus lucimarinus
FPAGLGVLVVDDDLLCLKVVEKMLKACKYKVTACSTAKTALEILRTRKEEFDIVLSDVHMPDMDGFKLLEIIQFELALPVLMMSANSDSSVVLRGIIHGAVDYLLKPVRIEELRNIWQHVVRRDYSSAKSSGSEDVEASSPSKRAKTSGSNSKSEEVDRTASEMSSGKARKKPTGKKGGKSVKEAEKKDVVDNSNSKKPRVVWSAELHAQFVTAVNQLGIDKAVPKRILDLMGVQGLTRENVASHLQKYRLYLKRLQGNDARGGGNASST

SEQ ID NO: 11 - protein, 941 aa, Chlamydomonas reinhardtii
  REGION 1..941: note = misc_feature - SGI1 polypeptide
MDSQGVKLEEHPGHTGGHWQGFPAGLRLLVVDDDPLCLKVVEQMLRKCSYEVTVCSNATTALNILRDKNTEYDLVLSDVYMPDMDGFRLLELVGLEMDLPVIMMSSNGDTSNVLRGVTHGACDYLIKPVRLEELRNLWQHVVRRRRQHAQEIDSDEQSQERDEDQTRNKRKADAAGVTGDQCRLNGSGSGGAAGPGSGGGAGGMTDEMLMMSGGENGSNKKARVVWSVEMHQQFVNAVNQLGIDKAVPKKILEIMGVDGSAGRLADTSGRDVCGTVYRLYLKRVSGVTPSGHHHNAAHKSNKPSPHTTPPPPALPGQAGTHPANQATAIPPPPQPGSGTAAGAGAAAAGTGGGAAAANGHAATTGAGTPGAAPGAGGGVGGTGAGGLGSGPDGAAAAAGPGPGAAVPGGLGGLPLPPGAGPGPGPGGFGGPSPPPPPHPAALLANPMAAAVAGLNQSLLNAMGSLGVGVGGMSPLGPVGPLGPLGGLPGLPGMQPPPLGMGGLQPGMGPLGPLGLPGMGGLPGLPGMNPMANLMQGMAAGMAAANQMNGMGGHMGGHMGGMNGPMGALAGMNGLNGAMMGGLPGMGGPQNMFQAAAAAAAQQQQQQQEQQHAMMQQAAAGLLASQQQQQQQQQQQQQQQALQQQQQQGMAVSPPGPHNATPNGQLHTHPQAHHPHQHGLHAHAHPHQHLNTAPAGALGLSPPQPPAGLLSASGLSSGPDGSGLGSGVGGLLDGLQQHPHHPQLQLAGSLGTGGTGRSSGAAGRGSLDLPADLMGMALLDFPPVPVPGGADVGMAGAGGGAAGAHHHGHQGHQGIGGGAGVGIAGGVGCGVPAAAHGLEPAILMDDPADLGAVFSDVMYGTPGGGGVPGGVPGGGVGLGLGAGQVPSGPAGAGGLHSHHHQHHHHQHHLGHVVPVGGVDPLAGDAAKMAMNDDDFFNFLLKN

SEQ ID NO: 12 - protein, 523 aa, unidentified
  REGION 1..523: note = misc_feature - SGI1 polypeptide
  source 1..523: note = Chromochloris zofingiensis
MDGFKLLETVGLELDLPVIMMSSNGEHTTVMRGVTHGACDFLIKPVRIEELRNIWQHVIRRTRHPVFRDLEPDDHEGGDYEASKKRKDLYRGENSSGSGGAGGLERDDDGSASKKPRVVWSVEMHQQFVQAVNQLGIDKAVPKKILELMNVDGLTRENVASHLQKYRLYLKRVQGVQAPFGLPNIQLPRQTSSKGAGSSSQQQHHQQQQHQQQHQHQHQTALGTGQQQSHQLQPCPVSTATPVMPSPDAMVAASMMSSQAMAAMAPGVMNPMTAMNSMMAGLNPNMMGMAAGLGLAGLGIGGMAGHPVPNPMLAGMGPMGLGLPPPPGMPPPPPGMPPGMPPGMPPGMPAMMQGLSMAGMSHLAAAGMRPPPGALGGHLGGPGLSPFGPPPPPGADPANMMANMSSMMANMQAALAFQADAAAAAQHQAASTGSVAPGRQQQVHQHQQAVGMAVDDAAAFPSPGCRPNGSADAGAQSAAEPNDFSRVFDDPFAQPAASPSGAAAAGSNEAPGMDDFLDFFLKS

SEQ ID NO: 13 - protein, 834 aa, unidentified
  REGION 1..834: note = misc_feature - SGI1 polypeptide
  source 1..834: note = Volvox carteri
MDGRAEGTVAIKQEDHASGHWHNFPAGLRLLVVDDDPLCLKVVEQMLRKCSYDVTTCTNATMALNLLRDKSTEYDLVLSDVYMPDMDGFKLLEVVGLEMDLPVIMMSSNGDTSNVLRGVTHGACDYLIKPVRLEELRNLWQHVVRRRRQLNLDMDSDEHSQERDDDQGRKRKADTAGCIGDQLRMMGAGCSGGANGLGSTGNLGAVATGSAGLGLGLGTAADELGLGLDNGSSKKARVVWSVEMHQQFVNAVNQLGIDKAVPKKILEIMNVDGLTRENVASHLQKYRLYLKRVSGAQQPGQNRVSRPSPPQPQSPQVPSQQQQSLPGGGGAAAAGAGQLQGGGGAAAAAASLASILAGGGPAGGGAGAGPPPGGGQLGADGGGPGPGLSSAVANAMSAAAAAGGFPTPPPPPPPHPAALLAANPMMAAAAGLNPLLGAMGGLGVGPLGPLNPLNGMPMPGMQPPLGLLPGLPGPGGQLGLGPLGPIGLPGPGPLPSLPAGLPLNPMANGLQQMAAANLMQGMAGMGQLPALSMNGMNGIMGPLPGVGLPGPQQHLFPQQQQPHLQQQQQQQQQKDLQMAQKQHQAAAAAAAVAAAVAAAQHQQQQPQAQQQPQPQQQQQQPGKLPQATVGTPALASPAGALPRQPSGQHPHTLSSSSLHTQQPHQQQLLHSQPSSTHLATNNTLAMAPALNGTLDVGGKGHLHAAGGQGAGAGAGAVLDIPPDLIGGLIEDGFGAPPGPTIQLAHGTAAVLDPTMLLDEGDNSDFAAVFQEMSSYGGGGVIGGGGSGAGAMGVLGHGLLAAGGPVMVDVAAGLAGVTETATRVDDDFLNFLLKS

SEQ ID NO: 14 - protein, 446 aa, Tetraselmis sp.
  REGION 1..446: note = misc_feature - SGI1 polypeptide 5172
MSCTVASFPPAAGGQGSPATPVPYQDLLVKRQDQWSNFPAGLRVLVADNDPASLQQVEKMLKKCSYQVTLCSSGKNSLEILRKRREEFDLVLADANLPDIDGFKLLHVCHTELSLPVVLMSGTSDTQLVMRGVMDGARDFLIKPLRVEELKVLWQHLVRFTSEITKTDAQLNVVKVELDGGRPAGEVSTSQNGSQCTEREGEGNSSKKQRMNWSDEMHQQFVNAVNQLGIDKAVPKRILDLMSVEGLTRENVASHLQKYRIYLKRMANHQENGKQAVMSTDTIARAEAAYQGGMPQGQQMMQQEHSGQAVQYSQPHAPGGLHQQAMPAQMHMGMMPAGPQPGSMQMAPHHVMQMPNGQVMVMQQMGPRPGMPPGMPQQMMASSQQMGMLQPGMPAGQMLHFQHPQQVHQHPPSSGPMHAVQHMEYAYSQPMQMAGWPVQGQPGNQA

SEQ ID NO: 15 - protein, 490 aa, Tetraselmis sp.
  REGION 1..490: note = misc_feature - SGI1 polypeptide 5185
MTPTPPMSCTVASFPPAAGGQGSPATPVPYQDLLVKRQDQWSNFPAGLRVLVADNDPASLQQVEKMLKKCSYQVTLCSSGKNSLEILRKRREEFDLVLADANLPDIDGFKLLHVCHTELSLPVVLMSGTSDTQLVMRGVMDGARDFLIKPLRVEELKVLWQHLVRFTSEITKTDAQLNVVKVELDGGRPAGEVSTSQNGSQCTEREGEGNSSKKQRMNWSDEMHQQFVNAVNQLGIDKAVPKRILDLMSVEGLTRENVASHLQKYRIYLKRMANHQENGKQAVMSTDTIARAEAAYQGGMPQGQQMMQQEHSGQAVQYSQPHAPGGLHQQAMPAQMHMGMMPAGPQPGSMQMAPHHVMQMPNGQVMVMQQMGPRPGMPPGMPQQMMASSQQMGMLQPGMPAGQMLHFQHPQQVHQHPPSSGPMHAGGEMIDPGSMQRLHQQPHYIGPNGQHMPAPAMGMPSGTVQHMEYAYSQPMQMAGWPVQGQPGNQA

SEQ ID NO: 16 - protein, 574 aa, Tetraselmis sp.
  REGION 1..574: note = misc_feature - SGI1 polypeptide 5230
MTMPLGGGLCMKDRIHGDERYRSKAKRQVNTIFAFTQRNTWRGRFRLCSYRTTELLGGSKTTEPGRGTFVLQIFMCVKNASIDDGSRHISTSRGLESVLKRRGGQGAPAAPVPYHDLLVKRQDQWSNFPAGLRVLVADNDPASLQQVEKMLKKCSYQVTLCSSGKNSLEILRKRREEFDLVLADANLPDIDGFKLLHVCHTELSLPVVLMSGTSDTQLVMRGVMDGARDFLIKPLRVEELKVLWQHLVRFTSEITKTDAQLNVVKVELDSGRPAGEVSTSQNGSQCAEREGEGNSSKKQRMNWSDEMHQQFVNAVNQLGIDKAVPKRILDLMSVEGLTRENVASHLQKYRIYLKRMANHQENGKQAVMSTDTIARAEAAYQGGMPQGQQMMQQEHSGQAVQYSQPHAPSGLHQQAMPAQMHMGMMPAGPQPGSMQMAPHHVMQMPNGQVMVMQQMGPRPGMPPGMPQQMMASSQQMGMLQPGMPAGQMLHFQHPQQVHQHPPSSGPMHAGGEMIDPGSMQRLHQQPHYIVPNAQHMPAPAMGMPPGAVQHMEYAYSQPMQMAGWPVQGQPGSQA

SEQ ID NO: 17 - protein, 674 aa, Oocystis sp.
  REGION 1..674: note = misc_feature - SGI1 polypeptide 5549
MLAFTHQRMTTAPALAVATSHFFAHVRVTTGSSAIATVFAARSRGSGLLAGFNTMENVKVEVPEVVPENVNFPAGLKVLVVDDDPLCLKVIDQMLRRCNYAATTCQSSLEALELLRSSKENHFDLVLSDVYMPDMDGFKLLEIIGLEMGLPVIMMSSNGETGVVFRGVTHGAVDFLIKPVRIEELRNLWQHVVRKTMVVPSNDKATSEEDGEESKHRVDRKRKESFHSRAREQVEIACSVVPALLWPTVPPSSVHPTSSSFLRSHVLLLQRSSGGKDVLDEGGSNAKKPRVVWSVEMHQQFVNAVNQLGIDKAVPKRILDLMNVDGLTRENVASHLQKYRLYLKRVAGINTATGSRNGKGRSDVSGLSGMPNGSLPMPGMMPPHMAAGMLLAGMAADVGPRPHPFPIMPMPAMALQGMHGGMAQMMQLPPGMPPPMMMPMAPLLPSQLAALGQQQQQQQQQQVARSESMPSENGVAGPSGSFTAMLNGPAPMESSPFAALQVFGPPQGMEQLTQQQQQQQQAGAAAFVAAFAAANGGDMQGGGGGPGPMLGGAGGAGPLLGGVGGGDPLHGGGGSSALGGRPMMSAEQPMGGSGGLASNSLTVQQNDLAQMCSQLDVNGLQAVAAAAAAGAMGAPGGAGGAMPPSSVGGVGPDMKLTEQDDFFSFLLKDSNLID

SEQ ID NO: 18 - protein, 488 aa, Micromonas sp.
  REGION 1..488: note = misc_feature - SGI1 polypeptide
  source 1..488: note = Micromonas sp. RCC299
MSTPAVSKGFPIGLRVLVVDDDPLCLKIVEKMLKRCQYEVTTFSRGAEALKTLRERKDDFDIVLSDVHMPDMDGFKLLEHIALELDIPVMMMSANCATDVVLRGIIHGAVDYLLKPVRIEELRNIWQHVVRRKRESSQGNLRSGEGGSNGRTVSGGSTGEGGGKDSKGSSEQHGDAKDKTGSAGGSGGSSKRKKGSGKKGDEGTDEVKDGSGGDENEDSSALKKPRVVWSAELHQQFVTAVNQLGIDKAVPKRILDLMGVQGLTRENVASHLQKYRLYLKRLQGVNSGGAPGGGPGFMSPIALDGSMVQGGPGGRVGSPAIGGPNGPIMVGHGHIDPAMLAGGAPQTIQMGMVYGGPGMGPPQMMAPNGKGGGGMPGGYVMQPGQMMAPNGQMMPVGQMGPGGMMVQGPGGGMMQMHDGGMMNGNGSYGSLQNMKQGNGVVMMPNGGMGGVDGAIPNMATGLINGQGLPDDDVLDMFLKDGLPEGEGF

SEQ ID NO: 19 - protein, 544 aa, Micromonas sp.
  REGION 1..544: note = misc_feature - SGI1 polypeptide
  source 1..544: note = Micromonas pusilla
MTAEKKELKVFPAGLRVLVVDDDPLCLRIVEKMLKRCQYEVTTFSRGAEALETLRARRDDFDIVLSDVHMPDMDGFKLLEHIALELDVPVMMMSANCATDVVLRGIIHGAVDYLLKPVRLEELRNIWQHVVRRQREPSKDGAAGKGGGASGAPEVSGDTHANTDDKQDGNATDSKGSGSQKRKSGKSGDDGGKDGGGSGGKDGDASNKGNNNKRKKGKSNDATETAGGAGVEDNDDTSGLKKPRVVWSPELHQQFVTAVNQLGIDKAVPKRILDLMGVQGLTRENVASHLQKYRLYLKRLQGVNNNGTVPSGAAGFMTGLAIDGVGGVMGPPTTGSPAMNGPGGPGGGLVMGPGHMGGPHMDGSGMMHMGPGGPMAGMTVVYGGGMPGGMPGGADSKNGASGQPPPGGYVVMGGPHGGGPGGAPMMMQHGGMVPGPGPGLVPGPGGSLMMPAGMMPDGGGGMVGVHVGPGVVMGQHQLGGKHSSGGAGMAGGSAAGKGAQRGGVGGAFDVPPTNGSLDADEIGDDVLTMFLKDGLPEMNDGDAL

SEQ ID NO: 20 - protein, 776 aa, Sphagnum fallax
  REGION 1..776: note = misc_feature - SGI1 polypeptide
MSGGDLSRVREGTADLDPVMASHQHPPPRQQSHQQPKNHQQEAHQQHCSSAETTSPNNTARGAGATYGKMEPADDFPAGLRILVVDDDPTCLAILKKMLQQCSYQVTTCGRATRALELLREDKDKFDLVISDVYMPDMDGFKLLELVGLEMDLPVIMMSGNGETSVVMKGITHGACDYLLKPVRIEELSNIWQHVVRKLRSEPKEHSASLEDGDRQRRGGAEDADNTSSAADTADGIWRNKKKKEAKEDEEDFEQDNDDPSTLKKPRVVWSVELHQQFVSAVNQLGIDKAVPKRILELMSVQGLTRENVASHLQKYRLYLKRLSGVTSQSNSLNVSFGGPDAGYGGLFGLDEMSDYRNLVTNGHLPAQTIAALHHANMAGRLGASSGMVGPSSPLDPSVLAQIAALQSGSLPRPGMDGSLQGNQAGLLQSLSGALDYNSLHQSHLLPAIGQLGQLDELPSLKSMQHQLGMGSLGGSTRNLAGSPNEELTMQLLQQRAQQQSGGSPINLPQATGILRPLSSNINQGGSVPNLVGVIPGTAIGLSNMCSGGREFGSSSGLLSASGSLMQSSTVEAQNLNFGGSSGSSGCSFQASVLSSKTGGLEDLNPAKRVRTTYSALSHSSPDLGQSSRPAWLGSQEGLVHGDPVYSPHQLSLPRQDIVGGIGSSGRPAYMGSQSMGSLGMNFPLSLAVDAGAVRPSLTRGQSLTEQVAANRELKFPKEERGRDNLMCARLGGGMITNESSSEELLNYLKQSHEGLGFMEGDLVSDGYPVDNLYVK

SEQ ID NO: 21 - protein, 715 aa, Physcomitrella patens
  REGION 1..715: note = misc_feature - SGI1 polypeptide
MGGGYLSSTVNMGESRDGGSPAMATLQQQQKHQPLNPNHQNPRNRSNSSPTNCYSNTAWGAKPAKLDTPDEFPVGMRVLVVDDNPTCLMILEQMLVRCAYRVTTCGKATEALSMLREDIGKFDVVISDVDMPDMDGFKLLELVGLEMDLPVIMVSGNGETSAVMKGITHGACDYLLKPVRIEELRNIWQHVVRKKRREVKAVATKSVEEAGGCERPKRGGGADDADYTSSATDTTDSNWKLTKRRKGEFKDENEEDNEQENDDPSTLKRPRVVWSVELHQQFVSAVNQLGIDKAVPKRILELMGVQGLTRENVASHLQKYRLYLKRLSGVTSQQGNMSAHFGGSDPFCMMPPDMSLANGQLTPQALAKFHMLGRMNATNGIGFSGGGLDPGMNQMFLQDLPRPPQLNSMLRNNTGLLASVPNGLQHLEQLSEPHHVHVVNELEHYPSNTKVYPQLNGNLDVSVGPLGAANGNLASNPNSDTLLMHILHSRASQQGVGSPSTLPQPRCGLNPTHLLSNDINFAPVGSLPNLAGSLGPAVGLSAIPGSAGGRDLSPSVGGSGASLSSPLGSLVRRPLMAEEQSNPVNSTNGTYSMAHSGQSPKPSGDTLPTPLNEGLEQQQPLWALYQNPMNQLSHGPSQGFPHDSLQWSVLTENLSFGDMGQSLSAGLISQFSSQGQDNGIGFAPPSQRGSYTRQSVSFPASSALDGRMVRSSYEP

SEQ ID NO: 22 - protein, 664 aa, Arabidopsis thaliana
  REGION 1..664: note = misc_feature - ARR2/SGI1 polypeptide
  source 1..664: note = Arabidopsis thaliana ARR2
MVNPGHGRGPDSGTAAGGSNSDPFPANLRVLVVDDDPTCLMILERMLMTCLYRVTKCNRAESALSLLRKNKNGFDIVISDVHMPDMDGFKLLEHVGLEMDLPVIMMSADDSKSVVLKGVTHGAVDYLIKPVRIEALKNIWQHVVRKKRNEWNVSEHSGGSIEDTGGDRDRQQQHREDADNNSSSVNEGNGRSSRKRKEEEVDDQGDDKEDSSSLKKPRVVWSVELHQQFVAAVNQLGVDKAVPKKILEMMNVPGLTRENVASHLQKYRIYLRRLGGVSQHQGNMNHSFMTGQDQSFGPLSSLNGFDLQSLAVTGQLPPQSLAQLQAAGLGRPTLAKPGMSVSPLVDQRSIFNFENPKIRFGDGHGQTMNNGNLLHGVPTGSHMRLRPGQNVQSSGMMLPVADQLPRGGPSMLPSLGQQPILSSSVSRRSDLTGALAVRNSIPETNSRVLPTTHSVFNNFPADLPRSSFPLASAPGISVPVSVSYQEEVNSSDAKGGSSAATAGFGNPSYDIFNDFPQHQQHNKNISNKLNDWDLRNMGLVFSSNQDAATATATAAFSTSEAYSSSSTQRKRRETDATVVGEHGQNLQSPSRNLYHLNHVFMDGGSVRVKSERVAETVTCPPANTLFHEQYNQEDLMSAFLKQEGIPSVDNEFEFDGYSIDNIQV

SEQ ID NO: 23 - protein, 1036 aa, Arabidopsis sp.
  REGION 1..1036: note = misc_feature - ARR18/SGI polypeptide
  source 1..1036: note = Arabidopsis halleri
LSKKQNEDASGRKEEDGKGNEHNGMESCTRMKRTVWTVELHQKFVNAFQQLGLDKASPEQIHALMNVEGLPVINVASHLQKYRLFLKKIYEGQQLDMATIQLLLSAGSHFPQTPWTNHCSSFIQQGHHQNSSNSSETYHTTLSPRVQKVNTFQPSSSPLKPLLFPKSNISAFKEDFKSIKEPAIVGDSSLDSSKPRNSFQTASKFPKTDPCTGSYIIEIMTEPYYGKSSRRHSNFSAYMGDFKSIKDPEIVQESRTRKNHGRVVWSHELHQKFLNAIDQLGGNEKAIPKKILAVMNVEGLTRLNVATHLQKYRQCCSAEAQQLNMATRKLPSSEHLPQSPSTNHHSSLSPRVQDVNIRLWSSSPKRQDQILVYVLFSFENDNGREETTCRRIASTMELGSTEDGRHDKFPVGMRVLAVDDNPTCLRKLEELLLRCKYHVTKTMESRKALELLRENSNMFDLVISDVEMPDTDGFKLLEIGLEMDLPVIMLSAHSDYDSVMKGIIHGACDYLVKPVGLKELQNIWHHVVKKNIKSYAKNIGPSRQLLPPSESNLVPSASKKRKEKASDSGDEDDSDREEDDGEGSEQDGEESGTRKKPRVVWSQELHQKFVSAVQQLGLDKAVPKKILDLMSIEGLTRENVASHLQKYRLYLKKIDEGQQQNMTPDAFGTRDSSYFQMAQLDGLRDFTATRQIPSSGLLSRSHLTKLQPPMYSSINLQGMNSSSFIQQGHHHNSSNSANPFGTYHTTLSPRIQNVNLLQRTSSPLETLQFPRSKSYIGDFKGIGDRAVGGSFLDSCMPFGSSSTSLPSASTNTLMLQANYTQPLHIASDGNQPCIEGTPSNSASPNISFQGLSRFPSHSWQGNLNTTRFPPSSLPLNQAFLPDQVTCAGNNLGDCTSLVSAGNPGGEMQCEPQLLGGFMQNMNPLDGQKWEQQNSMLNNPFGNIEYPLSADNMVFRDNNATRNKGLDESLMNPIDNSQEYVGKATTMLDPEMKSGKPENDNQHDVFDDIMNEMMKQEENNGMVSVATRFGFDSFPPP

SEQ ID NO: 24 - protein, 774 aa, Arabidopsis lyrata
  REGION 1..774: note = misc_feature - SGI1 polypeptide
MGDFKSIKEPEIVQESRTRKNHGRVVWSHELHQKFLHAIDQLGGNDKAIPKKILAVMNVEGLTRLNVATHLQKYRQCCSTEAQQLNMATRKLPSSEHLPQSPSTNHHSSLSPRVQDNDNGREETTCRRIASTMELGSTEDGRHDKFPVGMRVLAVDDNPTCLRKLEELLLRCKYHVTKTMESRKALELLRENSNMFDLVISDVEMPDTDGFKLLEIGLEMDLPVIMLSAHSDYDSVMKGIIHGACDYLVKPVGLKELQNIWHHVVKKNIKSYAKNIGPSRQLLPPSESNLVPSASKKRKEKANDSGDEDDSDREEDDGEGSEQDGDEAGTRKKPRVVWSQELHQKFVSAVQQLGLDKAVPKKILDLMSIEGLTRENVASHLQKYRLYLKKIDEGQQQNMTPDAFGTRDSSYFQMAQLDGLRDFTATRQIPSSGLLSRSHLTKLQPPMYSSINLQGMNSSSFIQQGHHHNSSNSANPFGTYHTTLSPRIQNVNLFQRTSSPLETLQFPRSKSYIGDFKGIGDRAVGGSFLDSCMPFGSSSTSLPSASTNTLMLQANYTQPLHISSDGNQPCIEGTPSNSASPNISFQGLSRFPSHSWQGNLNTTRFPPSSLPLNPAFLPDQVTCAGNNLGDCTSLVSAGNPGGEIQCEPQLLGGFMQNMNPLDGQKWEQQNCTMLNNPFGNIEYPLPADNMVFRDNNATRSKGLDESLMNPIDNSQEYVGKATTMLDPEMKSGKPENDNQHDVFDDLMNEMMKQEENNGMVSVATRFGFDSFPPP

SEQ ID NO: 25 - protein, 578 aa, unidentified
  REGION 1..578: note = misc_feature - SGI1 polypeptide
  source 1..578: note = Helianthus annus
MTTGSSFGSGSLGCKQETGVPDQFPAGLRVLVVDDDVICLKILEQMLRRCSYHVTTCSQATAALNLLRERKGCFDVVLSDVHMPDMDGFKLLELVGLEMDLPVIMMSADGRTNLVLRGIRHGACDYLIKPIREEQLKNIWQHVIRKKWNENKEHEHSGSVDDKDRHKRGGDDNDYASSVNEGGDGILTSHKKKRHNNKEEDDGELETDEPGGSKKARVVWSVELHQQFVTAVNQLGIDKAVPKRILELMNVPGLTRENVASHLQKFRLYLKRLSGVAQQGGGPNSFCGSIDQNPKLASYARFEIQALAASGQIPPQTLVALHAELLGQPTANVGMPVLDHQPLMQPSKCGPVDHVMSYGQTLPSNVTKQVPQPAIEDVHSGLGAWHSNNMVGGYGQLGGQNWHNMLLGMLQSQSHQLQKQSITVQPSRLVVPSQSSNFQAVNNGVPVNQTTGFNNSTVINYAVGQRTERDVENQIGGQSSVSNISVKEMGEKQISFGESVHVLDQGSLRNLGFVGKKSSIPSRFAVYEAAESLTHNLNYGDNNGERRVKQEPNIEFLENSKAGAHRVSQNDLMSKQVR

SEQ ID NO: 26 - protein, 428 aa, Vitis vinifera
  REGION 1..428: note = misc_feature - GSVIVT01011650001
MAALLKVPPQSSGGTNGSCKADVVVSDQFPAGLRVLVVDDDVTCLKILEQMLRRCLYHVTTCSQATIALNILREKKGCFDIVLSDVHMPDMDGYKLLEHVGLEMDLPVIMMSADGRTSAVMRGIRHGACDYLIKPIREEELKNIWQHVVRKKWNENKEHEHSGSLEDNDRHKRGGEDAEYASSVNEGAEGILKGQKKRRDSKDEDDGELENEDPSTSKKPRVVWSVELHQQFVSAVNQLGIDKAVPKRILELMNVPGLTRENVASHLQKFRLYLKRLSGVAQQQGGIPNSFCGPVEPNVKLGSLGRFDIQALAASGQIPPQTLAALQAELLGRPTSNLVLPAMDQPALLQASLQGPKCIPVEHGVAFGQPLVKCQTNISKHFPPTVVSTEDVPSGFGAWPSNSLGTVGTSGSLGGLSAQNNNILMDMK

SEQ ID NO: 27 - protein, 659 aa, unidentified
  REGION 1..659: note = misc_feature - SGI1 polypeptide
  source 1..659: note = Amborella trichopoda
MANVQKLPHSSISTASSYGSCRGEGVPDQFPAGLRVLVVDDDTTCLRILEQMLRKCMYKVTTCCRATDALDTLRGSKGCFDVVISDVYMPDMDGFKLLEHVGLEMDLPVIMMSADARFSAVMKGIKHGACDYLIKPVRIEELKNIWQHVVRKKWNETKEHDQSGSIEDNERHKRGSDDAEYASSVNEGTDGNWKVQKKRKDSKEEEDDGEQENEDPSAAKKPRVVWSVELHQQFVNAVNQLGIDKAVPKRILELMNVQGLTRENVASHLQKFRLYLKRLSGHQAGVSSSFCGSVDPNSKLGPLSQLDIRALTASGQIPSQTLAALQAELLGRPSNNVAMPVYGQTLVKCQPNLPKQFPQPNLPVDDVQSSLSIWQHHLSSGMPLGGLNPQNNGLLMQQQQQLTIESNRPCNVQPSCHVAPSNGGFTMRNNPTSSNASSVEYNSLLSSQGDVGQISQASGSDLATTVQSNGGFKSLDYRNMGQVSLESTSDLVSTQNNGFKGMELRNVGSLGGYPLSSSVSAGSTKTENGQSFSQVRTGPRMSMGPTGQFVGPPTIRRLPMVDGGTHRNSLGFVGKGVSIPSRFMPDSGSPTGVGEECTLPKQEVDPDFFDSLKVGPVGVQHYASGDLMSVLSKQQQASTGNLDCEFGIDGYQLGNIHVK

SEQ ID NO: 28 - protein, 669 aa, Ricinus communis
  REGION 1..669: note = misc_feature - SGI1 polypeptide
MAALQRVASSVSATASNYSSCKGNGVVTATADVAVSDQFPAGLRVLVVDDDTTCLRILEQMLRRCLYHVTTCSQAKVALNLLRERKGCFDVVLSDVHMPDMDGFKLLEHVGLEMDLPVIMMSADGRTSAVMRGIRHGACDYLIKPIREEELKNIWQHVVRKKWHENKEIEHSGSLEDNDRHKRGNEDAEYTSSVNEGTEGVLKGQKRRSNSKDEDDGEPDSDDPSTSKKPRVVWSVELHQQFVSAVNQLGIDKAVPKRILELMNVPGLTRENVASHLQKFRLYLKRLSGVAQQGGISSTFCGPMDSNVKLNSLGRFDIQALAASGQIPPQTLAALHAELFGRPTGSLVTTMDQPTLLQASRQSPKCIPVEHGVTFGQPIVKCSSGISKHFPQNMVSVEEVSSGYGAWPSNSLGTVGPSTNLGGMTTQNGNMLMDIFHQQQKQQQPQQQQSLADPSRSINVQPSCLVVPSQSSACFQAGNSPASVNQSNFNRNVVIDYSLLSSQSNNSALNIGHIPEGDLKTTGAVNGYSAPGSLSPPASSCSVNADSGVPRQVQNPTLAFGAVRQLPALSPNIFNIQGSYGVRSDDILDQGPFFKNLGFVGKGTCIPSRFAVDEFETPSSNLSHGKLYVENNDNKVKQEPNIDFTDTSRVGIPVLQQYPPNDLMSVFTE

SEQ ID NO: 29 - protein, 654 aa, Solanum lycopersicum
  REGION 1..654: note = misc_feature - Solyc04g008050.2.1
MVSMSGEVATCKSEATVVTDHFPVGLRVLVVDDDVVCLRIIEQMLRRCKYSVTTCTQAMVALNLLREKRGTFDIVLSDVHMPDMDGFKLLELVGLEMDLPVIMMSGDGRTNLVMRGVQHGACDYLIKPIRDEELKNIWQHVVRKRYNSSKEPECSGSLDDNDRYRRRSDDAECASSVIEGADGVLKPQKKKREAKEDDTEMENDDPSTTKKPRVVWSVELHQQFVSAVNQLGIDKAVPKRILELMNVPGLTRENVASHLQKFRLYLKRLSGVVQQQGGLPSTFCGPIEQNSELGSLGRFDIQALAASGQIPPETLTALHAELLGRSTSNLVLPAVEQQNLVQVSLQQAKCIPVDQVMAYGQPLLKCPASISNSKHLSQAILSAEDVHSGFGSQRAKNICMVPSSNPIAPNSNMLTAMMQQQQWQKQQQIELQHRQSGPPEVNRSINVQPSCLVLPSQLPGHFQVGDSPASISRAGSLSKSSVIDYGVLSPQSNNSSGVVQVLDRELKPECGLNRLPSGGSLSRSCSINADNSVDLQLHNSSSAFGSSKQLPGLIPSHLGSPVPYCINSSLVLDQGRMKGASIPSRFAVDESDSPMCNFNTAKIYLEETKVKQEPNMNVMENAKVGPAIFQKFQPGDLMSVFRLSFARVKVSSSP

SEQ ID NO: 30 - protein, 653 aa, Solanum tuberosum
  REGION 1..653: note = misc_feature - SGI1 polypeptide
MSGDVATCKSEATVVTDHFPLGLRVLVVDDDVVCLRIIEQMLRRCKYSVTTCTQAMVALNLLREKRGTFDIVLSDVHMPDMDGFKLLELVGLEMDLPVIMMSGDGRTNLVMRGVQHGACDYLIKPIRDEELKNIWQHVVRKRYNSSKELECSGSLDDNDRYKRGSDDAECASSVIEGADGVLKPQKKKREAKEEDDTEMENDDPSTSKKPRVVWSVELHQQFVSAVNQLGIDKAVPKRILELMNVPGLTRENVASHLQENQKFRLYLKRLSGVVQQQGGLPSTFCGPIEQNSELGSLGRFDIQALAASGQIPPETLTALHAELLGRSTSNLVLPAVEIQNLLQASLQQAKCIPADQVMAYGQPLLKCHPSISNSKHLSQSILSAEDVHSGFGSQRAKNICLVPSSNPIGLAAPNSNMLMAMMQQQQWQKQQQMELQHRRSGPPEVNHSINVQPSCLVLPSQLPGNFQVGDSPASISRAGSLSKSSVIDYGVLSPQSNNSSGVVQVLDRELKPECGLNRLPSGGSLSRSCSINADNSVGLQLHNSSSAFGSSKQLPALIPNHLGSPVPYYINSSQVLDQGHTRNPGVGKCASIPSRFAVDESDSPMCNFNTAKNYLEETKVKQEPNMNVMENAKVGPAIFQKFQPGDLMSVFSD

SEQ ID NO: 31 - protein, 669 aa, Gossypium hirsutum
  REGION 1..669: note = misc_feature - SGI1 polypeptide
MATMHRVVQSSVSTSDATTTSYDGLTSCKAADIVISDQFPAGLRVLVVDDDITCLKILEKMLHRCRYHVTTCPQAKVALNLLRERKGCFDVILSDVYMPDMDGYKLLEHVGLEMDLPVIMMSADGSTRAVMKGIRHGACDYLIKPIREEELKNIWQHVVRKKWNENKELEHSGSLDDTDQHKQRHDDAEYASSVNDATETSLKPLKKRSNSKEEDDGEIDNDDPSTSKKPRVVWSVELHQQFVSAVNQLGIDKAVPKRILELMNVPGLTRENVASHLQKFRLYLKRISGVAQQGGIANPLCGPVEANVKIGSLGSFNIQALAASGQIPPQTLAAVHAELLGRSAGNLVVATDQPALLQATPQGAKCIQVDQGVAFVQHSVKSESSSSKHFSQSFAPVEDVASGFRSWPSNNIGTAGPSNSGGLSSQNGNMLIDLLQQQQQLQKPQQRSTVSELRRSINVQPSCHVVPSQSSASFRAGNSPVSVTQNGSYSRTAVIDYSLLSSQSNCPSLNIGQVSDVNLQTTGVLSGYIPPASVSPSVSSCSVNADNCASQQVQTSSMTFKASRHLPGFVHSTSNIPDPYGSTKSGDLLNQEPFNNLGYINKGTCLPAKFAVDEFQSHLSSSSHGKVFSENIGTRVKQEPSMEFGDNAKVGIPMLQQFRPNDLMSVFTE

SEQ ID NO: 32 - protein, 681 aa, Theobroma cacao
  REGION 1..681: note = misc_feature - SGI1 polypeptide
MNSSSGKGSMSAASSSAAWKAGDVVPDQFPAGLRVLVVDDDPTCLMILEKMLRTCLYEVTKCNRAETALSLLRENKNGFDIVISDVHMPDMDGFKLLEHIGLEMDLPVIMMSADDGKHVVMKGVTHGACDYLIKPVRIEALKNIWQHVVRKRKNEWKDFEQSGSVEEGDRQPKQSEEADYSSSANEGNWKSSKKRKDDDDEAEERDDTSTLKKPRVVWSVELHQQFVAAVNQLGIDKAVPKKILELMNVPGLTRENVASHLQKYRLYLRRLSGVSQHQSNLNNSFMSPQEATFGPLSPLNGLDLQTLAATGQLPAQSLATFQAAGLGRSTAKSGIAMPLVDQRNIFSFENPKLRFGEGQQQHMNNNKQLNLLHGIPTTMEPKQLASLHHSAQSIGNINMQVTSHGVQGSQNNSLLIQMAQPQPRGQILNDSTGSHAPRLPSTLGQPILSNGIAANVSTRNGIPENIRGPGYNPVSQTSSLLNFPMNHTSELPGNSFPLGTTPGISSLTSKGAFQEDINSDVKGSGGFMPSYDIFNDLNQHKPQNWELQNVGMTFDASQHSNSLQGNLDLAQSILVQQGFSSGQMNGQNRSAAVVSKAMFSAGDCTEQGNAQNVNHHLNNLLVDNTIRIKSERVADAGPANLFPDHFGQEDLMSALLKQQDGIAPAENEFDFDGYSMDNIPV

SEQ ID NO: 33 - protein, 579 aa, Phaseolus vulgaris
  REGION 1..579: note = misc_feature - SGI1 polypeptide
MNLSNGKGSMSTVTTTAVMKSGDAVSDQFPAGLRVLVVDDDPTCLMILEKMLRTCLYEVTKCNRAETALSLLRENKNGFDIVSANEGSWRNSKKRRDEEEEAEDRDDTSTLKKPRVVWSVELHQQFVAAVDQLGIDKAVPKKILELMNVPGLTRENVASHLQKYRLYLRRLSGVSQHQNNLNNSFLGSQEATFGTISSINGIDLQTLAVTGQLPAQSLATLQAAGLGRSTAKTGVPMPLMDQRNLFSFENPRVRFGEGQQQHLSSSKPMNLLLGIPTNMEPKQLANLHQSTQSIASLNMRVNASATQGNPLMMQMPQSQPRGQMLSENTGPRVPRLPSSLGQPTVSNGISNGFLGRNGIAGNNRGPAYNPVPPNSSLLSFPMNQSSEVSVNNSLPLGSSPGISSITTKGSFQEEVTSGIKATGGFPSYDIFNELHHQKSHDWEITNPSLTYSASHHANPLQGNIDVSPSVLVHQGFSSTQQNGQSRDATLIGKAMFSLGEGSEQDNLQNAVQHLHPLLVDNSIRVKAERIPDASSQTNLFPDHYVQEDLMSALLKQQEGMGPAESEFEFDAYSLDNIPV

SEQ ID NO: 34 - protein, 679 aa, Glycine max
  REGION 1..679: note = misc_feature - SGI1 polypeptide
MNLSNGKGSMSTLTASVVMKSGDAVSDQFPAGLRVLVVDDDPTCLMILEKMLRTCLYEVTKCNRAETALSLLRENKNGFDIVISDVHMPDMDGFKLLEHIGLEMDLPVIMMSADDGKSVVMKGVTHGACDYLIKPVRIEALKNIWQHVVRKRKNEWKDAEQSGSAEEGDRQPKASDEADYSSSANEGSWRNSKKRRDEEEEAEDRDDTSTLKKPRVVWSVELHQQFVAAVDQLGIDKAVPKKILELMNVPGLTRENVASHLQKYRLYLRRLSGVSQHQNNMNNSFLSPQEATFGTISSINGIDLQTLAVAGQLPAQSLATLQAAGLGRPTGKAGVPMPLMDQRNLFSFENPRLRFGEGQQQHLSTSKPMNLLHGIPTNMEPKQLANLHQSTQSIGSLNMRVNASATQGSPLLMQMAQSQPRGQMLSENIGPRVPRLPSSLGQPTVSNGISNGLLGRNGIAGNNRGPAYNPVPPSSSLLSFPMNQTSEMSVNNSFPLGSTPGISSITTKGSFQEEVTSGIKGSGGFPSYDIFNELHHQKPHDWEITNPNLTYNASQHANPLQGNIDVTPSVLVHQGFSSTQQTGQSRDAALIGKAMFSMGEGLEQNNFQNASQNLNSLLLDNSIRVKAERIPDASSQTNLFPEHYGQEDLMSALLKQQEGMGPSENEFDFDGYSLDNIPV

SEQ ID NO: 35 - protein, 668 aa, Chenopodium quinoa
  REGION 1..668: note = misc_feature - SGI1 polypeptide
MNLGGGLMGSMAMPSSTVSRKSSEVVTADQFPVGLRVLVVDDDPTCLTILEKMLRTCRYEVTKTNRAEHALNMLRENKNGFDVVISDVHMPDMDGFKLLEQVGLEMDLPVIMMSADDSKQVVMKGVTHGACDYLIKPVRIEALKNIWQHVVRKKKYEYNKDVEQSGSWDEGDRQLKHDDAVSSPANDGSWKNSKRKSGEDDEADDKDDTTTLKKPRVVWSVELHQQFVAAVNQLGIDKAVPKKILELMNVPGLTRENVASHLQKYRLYLRRLSGVSQHQGGLNSSFMPQDPSFSTMSSLGGIDLQTLAATGQLSAQTLAAYTRLPPTIKPGISMPFVDQRNLFSFENSKLRYGDGQQSQISNVSKQMNLLHGFPTTMEPKQLAVLNQSAQTLGSMNMQANASSSHQSSSLLMQQMVPQQRGHISNESISSQVPRIQPSVGQPLQSNGNANAVLSRNGIPYDPVNQSASVVDFSVNHIPELPGNSFPLGSTPGITSITSKGFNQEEIGSDIKVSRGFVGSYDMFSELQHKPQEWQMQNPNMGFAGSSQHVPSVQSGVNVAPSIMVNQSYVSGQKNEQNGHSMAGKPMYSAGLENQHMGMQNVNQNYNSIHVNNSSRVKAESVSDVVNLGANLFDYSPEDMLSTIMLKQQEGIGSGDFDFDGYTLDNIPV

SEQ ID NO: 36 - protein, 670 aa, Malus domestica
  REGION 1..670: note = misc_feature - SGI1 polypeptide
  VARIANT 195: note = misc_feature - Xaa can be any naturally occurring amino acid
  VARIANT 215: note = misc_feature - Xaa can be any naturally occurring amino acid
  VARIANT 530: note = misc_feature - Xaa can be any naturally occurring amino acid
  VARIANT 540: note = misc_feature - Xaa can be any naturally occurring amino acid
  VARIANT 579: note = misc_feature - Xaa can be any naturally occurring amino acid
MAALQRVAQSSVSTTASSYGSCKVGGGVLSPSAGIEMAVPNQFPAGLRVLVVDDDTTCLRILELMLLRCLYQVTTCSEATVALNLLRERKDCFDVVLSDVHMPDMDGFKLLEHVGLEMDLPVIMMSADGRTSVVMRGIRHGACDFLIKPISEAELKNIWQHVVRKKWNGSKELEHSGSLEDNDPHKRGNNDFEYXSSVNEGTEVSLKGHKKRINXKEDDDGDTENDDLSTSKKPRVVWSVELHQQFVTAVNQLGLDKAVPKRILELMNVPGLTRENVASHLQKFRLYLKRLSGVAQQQSGIANPLCGPVDSNGKLGSLSRFDFQALAASGQIPPQTLAALQAELLGQPAGNLVPAMDQPALLHASLQAPKRPPVEHGVPFMQPFVKSQSNVSKHFPQSVISAEDASLGFGQWRSNSRSTVAPSNDHGGLSTQNSNLLMGIVPQEQRQHKRTQQQSVLTEPSRSFNVQPSCLVVPSQSSTGFQAGNSPASVNQSSSFNRSTVVDYSLPSDQSNNSLNVGHIPTGNPKTSGXLGGYSGPGSXCATSCLVNADNSTSYQNSTATFSDSRELPGFLHNTANSXGFYVDKSGEMLDQGPLRNLGFVGKETCIPSRFAVDDFESQMSNLNPGRIHVESSGTLVKQEPSEDYVDNAKLGIPILHQYSSSDFMSPFAD

SEQ ID NO: 37 - protein, 802 aa, Zea mays
  REGION 1..802: note = misc_feature - SGI1 polypeptide
PYPTHTLLPQPHLSLSACVLLVLLSLSSPALTSPPFPAVSWISRIQTTALVSLPSCLLPAYVQEGPCLGDPGAWFLGSAASAAVGFAEPEPPEMTVDELKLQARASGGHGAKDQFPVGMRVLAVDDDPTCLKILENLLLRCQYHVTTTGQAATALKLLREKKDQFDLVISDVHMPDMDGFKLLELVGLEMDLPVIMLSANGETQTVMKGITHGACDYLLKPVRIEQLRTIWQHVVRRRSCDAKNSGNDNDDSGKKLQVVSAEGDNGGVNRNKRISRKGRDDNGDDGDDSDDNSNENGDSSSQKKPRVVWSVELHRKFVAAVNQLGIDKAVPKKILDLMNVENITRENVASHLQKYRLYLKRLSADASRQANLTAAFGGRNPAYVNMGLDAFRQYNAYGRYRPVPTTNHSQPNNLLARMNSPAFGMHGLLPSQPLQIGHNQNNLSTSLGNVGGMNNGNLIRGAHMPLQDTSKCFPTGPSGNSFANISNSTQLVTTNNLPLQSLEPSNQQHLGRLHSSADPFNSFVGEPPQFADLGRCNTTWPTAVSSSNVQEIGQKDRIVNRPKLEPLSSFTEASSQIPLLGNEMQSHQVASLASNGLPMPFTQEAVPFAYGSSTNSREMLNNNLALSNSGVNSTLPNLRIDGSVVPGQTLGGSNSGGCVVPPLQDGRIDHQAVSSHLNYNNELMGTGRLQRGLSGGLDDIVVDMFRPDRADDGVSFIDGDWELRPGSSVTSEYQLCGICYLNSYDYVFKSGVNCGYRDIQHVYEPRNDVLFPLGNRFAVPFVDCHCIVASLAETEVKGKDQA

SEQ ID NO: 38 - protein, 591 aa, Brassica rapa
  REGION 1..591: note = misc_feature - SGI1 polypeptide
MLNPGVVGGSSNSDPFPSGLRVLVVDDDPTCLMILERMLKTCLYRVTKCNRAEIALSLLRKNKNGFDIVISDVHMPDMNGFKLLEHVGLEMDLPVIMMSADDSKSVVLKGVTHGAVDYLIKPVRIEALKNIWQHVVRKKQNVSEHSGSVEETGGDRQQQQRGDDDDDGNNSSSGNNEGNLRKRKEEEQGDDKEDTSSLKKPRVVWSVELHQQFVAAVNHLGVDKAVPKKILEMMNVQGLTRENVASHLQKYRIYLKRLGGVSQGNMNHSFLTGQDPSYGPLNGFDLQGLATAGQLQAQSLAQLQAVGLGQSSSPLIKPGITSVDQRSFFTFQNSKSRFGDGHGPMMMNGGGGNKQTSLLHGVPTGHMRLQQQQMAGMRVAGPSMQQQQQQSMLSRRSVPETRSSRVLPAATHSALNNSFPLASAPGMMSVSDTKGVNEFCNPSYDILNNFPQQQHHNNNNNRVNEWDLRNVGMVFNSHQDNTTSAAFSTSEAYSSSSTHKRKREAELVVEHGQNQQQPQSRSVKPMNQTYMDGGGSVRMKTETVTCPPQATTMFHEQYSNQDDLLSDLLKQEGLLDTEFDFEGYSFDSILV

SEQ ID NO: 39 - protein, 691 aa, Oryza sativa
  REGION 1..691: note = misc_feature - SGI1 polypeptide
MAPVEDGGGVEFPVGMKVLVVDDDPTCLAVLKRMLLECRYDATTCSQATRALTMLRENRRGFDVIISDVHMPDMDGFRLLELVGLEMDLPVIMMSADSRTDIVMKGIKHGACDYLIKPVRMEELKNIWQHVIRKKFNENKEHEHSGSLDDTDRTRPTNNDNEYASSANDGAEGSWKSQKKKRDKDDDDGELESGDPSSTSKKPRVVWSVELHQQFVNAVNHLGIDKAVPKKILELMNVPGLTRENVASHLQKFRLYLKRIAQHHAGIANPFCPPASSGKVGSLGGLDFQALAASGQIPPQALAALQDELLGRPTNSLVLPGRDQSSLRLAAVKGNKPHGEREIAFGQPIYKCQNNAYGAFPQSSPAVGGMPSFSAWPNNKLGMADSTGTLGGMSNSQNSNIVLHELQQQPDAMLSGTLHSLDVKPSGIVMPSQSLNTFSASEGLSPNQNTLMIPAQSSGFLAAMPPSMKHEPVLATSQPSSSLLGGIDLVNQASTSQPLISAHGGGNLSGLVNRNPNVVPSQGISTFHTPNNPYLVSPNSMGMGSKQPPGVLKTENSDALNHSYGYLGGSNPPMDSGLLSSQSKNTQFGLLGQDDITGSWSPLPNVDSYGNTVGLSHPGSSSSSFQSSNVALGKLPDQGRGKNHGFVGKGTCIPSRFAVDEIESPTNNLSHSIGSSGDIMSPDIFGFSGQM

SEQ ID NO: 40 - protein, 126 aa, Parachlorella sp.
  REGION 1..126: note = misc_feature - Response Regulator receiver domain of SGI1 polypeptide of SEQ ID NO:3
PAGLKVLVVDDDLMCLKVVSAMLKRCSYQVATCSSGSEALTLLRERNEDGSSDQFDLVLSDVYMPDMDGFKLLEHIGLELELPVIMMSSNGDTNVVLRGVTHGAVDFLIKPVRIEELRNVWQHVVR

SEQ ID NO: 41 - protein, 123 aa, unidentified
  REGION 1..123: note = misc_feature - Response Regulator receiver domain of SGI1 polypeptide of SEQ ID NO:9
  source 1..123: note = Coccomyxa subellipsoidea
PAGLKVLVVDDDPLCLKVVEHMLRRCNYQVTTCPNGKAALEKLRDRSVHFDLVLSDVYMPDMDGFKLLEHIGLELDLPVIMMSSNGETNVVLRGVTHGAVDFLIKPVRVEELRNVWQHVVRRK

SEQ ID NO: 42 - protein, 122 aa, unidentified
  REGION 1..122: note = misc_feature - Response Regulator receiver domain of SGI1 polypeptide of SEQ ID NO:10
  source 1..122: note = Ostreococcus lucimarinus
PAGLGVLVVDDDLLCLKVVEKMLKACKYKVTACSTAKTALEILRTRKEEFDIVLSDVHMPDMDGFKLLEIIQFELALPVLMMSANSDSSVVLRGIIHGAVDYLLKPVRIEELRNIWQHVVRR

SEQ ID NO: 43 - protein, 123 aa, Chlamydomonas reinhardtii
  REGION 1..123: note = misc_feature - Response Regulator receiver domain of SGI1 polypeptide of SEQ ID NO:11
PAGLRLLVVDDDPLCLKVVEQMLRKCSYEVTVCSNATTALNILRDKNTEYDLVLSDVYMPDMDGFRLLELVGLEMDLPVIMMSSNGDTSNVLRGVTHGACDYLIKPVRLEELRNLWQHVVRRR

SEQ ID NO: 44 - protein, 61 aa, unidentified
  REGION 1..61: note = misc_feature - Response Regulator receiver domain of SGI1 polypeptide of SEQ ID NO:12
  source 1..61: note = Chromochloris zofingiensis
MDGFKLLETVGLELDLPVIMMSSNGEHTTVMRGVTHGACDFLIKPVRIEELRNIWQHVIRR

SEQ ID NO: 45 - protein, 123 aa, unidentified
  REGION 1..123: note = misc_feature - Response Regulator receiver domain of SGI1 polypeptide of SEQ ID NO:13
  source 1..123: note = Volvox carteri
PAGLRLLVVDDDPLCLKVVEQMLRKCSYDVTTCTNATMALNLLRDKSTEYDLVLSDVYMPDMDGFKLLEVVGLEMDLPVIMMSSNGDTSNVLRGVTHGACDYLIKPVRLEELRNLWQHVVRRR

SEQ ID NO: 46 - protein, 121 aa, Tetraselmis sp.
  REGION 1..121: note = misc_feature - Response Regulator receiver domain of SGI1 polypeptides of SEQ ID NO:14, SEQ ID NO:15, and SEQ ID NO:16
PAGLRVLVADNDPASLQQVEKMLKKCSYQVTLCSSGKNSLEILRKRREEFDLVLADANLPDIDGFKLLHVCHTELSLPVVLMSGTSDTQLVMRGVMDGARDFLIKPLRVEELKVLWQHLVR

SEQ ID NO: 47 - protein, 123 aa, Oocystis sp.
  REGION 1..123: note = misc_feature - Response Regulator receiver domain of SGI1 polypeptide of SEQ ID NO:17
PAGLKVLVVDDDPLCLKVIDQMLRRCNYAATTCQSSLEALELLRSSKENHFDLVLSDVYMPDMDGFKLLEIIGLEMGLPVIMMSSNGETGVVFRGVTHGAVDFLIKPVRIEELRNLWQHVVRK

SEQ ID NO: 48 - protein, 123 aa, Micromonas sp.
  REGION 1..123: note = misc_feature - Response Regulator receiver domain of SGI1 polypeptide of SEQ ID NO:18
  source 1..123: note = Micromonas sp. RCC299
PIGLRVLVVDDDPLCLKIVEKMLKRCQYEVTTFSRGAEALKTLRERKDDFDIVLSDVHMPDMDGFKLLEHIALELDIPVMMMSANCATDVVLRGIIHGAVDYLLKPVRIEELRNIWQHVVRRK

SEQ ID NO: 49 - protein, 123 aa, Micromonas sp.
  REGION 1..123: note = misc_feature - Response Regulator receiver domain of SGI1 polypeptide of SEQ ID NO:19
  source 1..123: note = Micromonas pusilla
PAGLRVLVVDDDPLCLRIVEKMLKRCQYEVTTFSRGAEALETLRARRDDFDIVLSDVHMPDMDGFKLLEHIALELDVPVMMMSANCATDVVLRGIIHGAVDYLLKPVRLEELRNIWQHVVRRQ

SEQ ID NO: 50 - protein, 123 aa, Sphagnum fallax
  REGION 1..123: note = misc_feature - Response Regulator receiver domain of SGI1 polypeptide of SEQ ID NO:20
PAGLRVLVVDDDPICLMILDRMLRQCSYQVTTCGRATKALELLREVKDKFDLVISDVYMPDMDGFKLLELVGLEMDLPVIMMSSDGETSAVMKGITHGACDYLLKPVRLEELRNIWQHVVRKK

SEQ ID NO: 51 - protein, 123 aa, Physcomitrella patens
  REGION 1..123: note = misc_feature - Response Regulator receiver domain of SGI1 polypeptide of SEQ ID NO:20
PVGMRVLVVDDNPTCLMILEQMLVRCAYRVTTCGKATEALSMLREDIGKFDVVISDVDMPDMDGFKLLELVGLEMDLPVIMVSGNGETSAVMKGITHGACDYLLKPVRIEELRNIWQHVVRKK

SEQ ID NO: 52 - protein, 123 aa, Arabidopsis thaliana
  REGION 1..123: note = misc_feature - Response Regulator receiver domain of SGI1 polypeptide of SEQ ID NO:22
PANLRVLVVDDDPTCLMILERMLMTCLYRVTKCNRAESALSLLRKNKNGFDIVISDVHMPDMDGFKLLEHVGLEMDLPVIMMSADDSKSVVLKGVTHGAVDYLIKPVRIEALKNIWQHVVRKK

SEQ ID NO: 53 - protein, 121 aa, Arabidopsis sp.
  REGION 1..121: note = misc_feature - Response Regulator receiver domain of SGI1 polypeptide of SEQ ID NO:24
  source 1..121: note = Arabidopsis halleri
PVGMRVLAVDDNPTCLRKLEELLLRCKYHVTKTMESRKALELLRENSNMFDLVISDVEMPDTDGFKLLEIGLEMDLPVIMLSAHSDYDSVMKGIIHGACDYLVKPVGLKELQNIWHHVVKK

SEQ ID NO: 54 - protein, 123 aa, Vitis vinifera
  REGION 1..123: note = misc_feature - Response Regulator receiver domain of SGI1 polypeptide of SEQ ID NO:26
PAGLRVLVVDDDVTCLKILEQMLRRCLYHVTTCSQATIALNILREKKGCFDIVLSDVHMPDMDGYKLLEHVGLEMDLPVIMMSADGRTSAVMRGIRHGACDYLIKPIREEELKNIWQHVVRKK

SEQ ID NO: 55 - protein, 123 aa, Glycine max
  REGION 1..123: note = misc_feature - Response Regulator receiver domain of SGI1 polypeptide of SEQ ID NO:34
PAGLRVLVVDDDPTCLMILEKMLRTCLYEVTKCNRAETALSLLRENKNGFDIVISDVHMPDMDGFKLLEHIGLEMDLPVIMMSADDGKSVVMKGVTHGACDYLIKPVRIEALKNIWQHVVRKR

SEQ ID NO: 56 - protein, 123 aa, Zea mays
  REGION 1..123: note = misc_feature - Response Regulator receiver domain of SGI1 polypeptide of SEQ ID NO:37
PVGMKVLVVDDDPTCLVVLKRMLLECRYDVTTCPQATRALTMLRENRRGFDVIISDVHMPDMDGFRLLELVGLEMDLPVIMMSADSRTDIVMKGIKHGACDYLIKPVRMEELKNIWQHVVRKK

SEQ ID NO: 57 - protein, 123 aa, Oryza sativa
  REGION 1..123: note = misc_feature - Response Regulator receiver domain of SGI1 polypeptide of SEQ ID NO:39
PVGMKVLVVDDDPTCLAVLKRMLLECRYDATTCSQATRALTMLRENRRGFDVIISDVHMPDMDGFRLLELVGLEMDLPVIMMSADSRTDIVMKGIKHGACDYLIKPVRMEELKNIWQHVIRKK

SEQ ID NO: 58 - protein, 60 aa, Parachlorella sp.
  REGION 1..60: note = misc_feature - Myb domain of SGI1 polypeptide of SEQ ID NO:3
KKPRVVWSVEMHQQFVNAVNSLGIDKAVPKRILDLMNVEGLTRENVASHLQKYRLYLKRV

SEQ ID NO: 59 - protein, 51 aa, unidentified
  REGION 1..51: note = misc_feature - Myb domain of SGI1 polypeptide of SEQ ID NO:9
  source 1..51: note = Coccomyxa subellipsoidea
KKARVVWSVEMHQQFVQAVNQLGIDKAVPKRILDLMNVDGLTRENVASHLQ

SEQ ID NO: 60 - protein, 61 aa, unidentified
  REGION 1..61: note = misc_feature - Myb domain of SGI1 polypeptide of SEQ ID NO:10
  source 1..61: note = Ostreococcus lucimarinus
KKPRVVWSAELHAQFVTAVNQLGIDKAVPKRILDLMGVQGLTRENVASHLQKYRLYLKRLQ

SEQ ID NO: 61 - protein, 65 aa, Chlamydomonas reinhardtii
  REGION 1..65: note = misc_feature - Myb domain of SGI1 polypeptide of SEQ ID NO:11
KKARVVWSVEMHQQFVNAVNQLGIDKAVPKKILEIMGVDGSAGRLADTSGRDVCGTVYRLYLKRV

SEQ ID NO: 62 - protein, 61 aa, unidentified
  REGION 1..61: note = misc_feature - Myb domain of SGI1 polypeptide of SEQ ID NO:12
  source 1..61: note = Chromochloris zofingiensis
KKPRVVWSVEMHQQFVQAVNQLGIDKAVPKKILELMNVDGLTRENVASHLQKYRLYLKRVQ

SEQ ID NO: 63 - protein, 60 aa, unidentified
  REGION 1..60: note = misc_feature - Myb domain of SGI1 polypeptide of SEQ ID NO:13
  source 1..60: note = Volvox carteri
KKARVVWSVEMHQQFVNAVNQLGIDKAVPKKILEIMNVDGLTRENVASHLQKYRLYLKRV

SEQ ID NO: 64 - protein, 60 aa, Tetraselmis sp.
  REGION 1..60: note = misc_feature - Myb domain of SGI1 polypeptides of SEQ ID NO:14, SEQ ID NO:15, and SEQ ID NO:16
KKQRMNWSDEMHQQFVNAVNQLGIDKAVPKRILDLMSVEGLTRENVASHLQKYRIYLKRM

SEQ ID NO: 65 - protein, 60 aa, Oocystis sp.
  REGION 1..60: note = misc_feature - Myb domain of SGI1 polypeptide of SEQ ID NO:17
KKPRVVWSVEMHQQFVNAVNQLGIDKAVPKRILDLMNVDGLTRENVASHLQKYRLYLKRV

SEQ ID NO: 66 - protein, 61 aa, Micromonas sp.
  REGION 1..61: note = misc_feature - Myb domain of SGI1 polypeptide of SEQ ID NO:18
  source 1..61: note = Micromonas sp. RCC299
KKPRVVWSAELHQQFVTAVNQLGIDKAVPKRILDLMGVQGLTRENVASHLQKYRLYLKRLQ

SEQ ID NO: 67 - protein, 61 aa, Micromonas sp.
  REGION 1..61: note = misc_feature - Myb domain of SGI1 polypeptide of SEQ ID NO:19
  source 1..61: note = Micromonas pusillada
KKPRVVWSPELHQQFVTAVNQLGIDKAVPKRILDLMGVQGLTRENVASHLQKYRLYLKRLQ

SEQ ID NO: 68 - protein, 60 aa, Sphagnum fallax
  REGION 1..60: note = misc_feature - Myb domain of SGI1 polypeptide of SEQ ID NO:20
KKPRVVWSVELHQQFVSAVNQLGIDKAVPKKILELMSVHGLTRENVASHLQKYRLYLRRL

SEQ ID NO: 69 - protein, 60 aa, Physcomitrella patens
  REGION 1..60: note = misc_feature - Myb domain of SGI1 polypeptide of SEQ ID NO:21
KRPRVVWSVELHQQFVSAVNQLGIDKAVPKRILELMGVQGLTRENVASHLQKYRLYLKRL

SEQ ID NO: 70 - protein, 61 aa, Arabidopsis thaliana
  REGION 1..61: note = misc_feature - Myb domain of SGI1 polypeptide of SEQ ID NO:22
KKPRVVWSVELHQQFVAAVNQLGVDKAVPKKILEMMNVPGLTRENVASHLQKYRIYLRRLG

SEQ ID NO: 71 - protein, 61 aa, Arabidopsis sp.
  REGION 1..61: note = misc_feature - Myb domain of SGI1 polypeptides of SEQ ID NO:23 and SEQ ID NO:24
  source 1..61: note = Arabidopsis halleri
KKPRVVWSQELHQKFVSAVQQLGLDKAVPKKILDLMSIEGLTRENVASHLQKYRLYLKKID

SEQ ID NO: 72 - protein, 60 aa, Vitis vinifera
  REGION 1..60: note = misc_feature - Myb domain of SGI1 polypeptide of SEQ ID NO:26
KKPRVVWSVELHQQFVSAVNQLGIDKAVPKRILELMNVPGLTRENVASHLQKFRLYLKRL

SEQ ID NO: 73 - protein, 60 aa, Glycine max
  REGION 1..60: note = misc_feature - Myb domain of SGI1 polypeptide of SEQ ID NO:34
KKPRVVWSVELHQQFVAAVDQLGIDKAVPKKILELMNVPGLTRENVASHLQKYRLYLRRL

SEQ ID NO: 74 - protein, 60 aa, Zea mays
  REGION 1..60: note = misc_feature - Myb domain of SGI1 polypeptide of SEQ ID NO:37
KKPRVVWSVELHQQFVNAVNHLGIDKAVPKKILELMNVPGLTRENVASHLQKFRLYLKRI

SEQ ID NO: 75 - protein, 60 aa, Oryza sativa; identical to SEQ ID NO: 74
  REGION 1..60: note = misc_feature - Myb domain of SGI1 polypeptide of SEQ ID NO:39

SEQ ID NO: 76 - DNA, 5607 bp, synthetic construct
  misc_feature 1..5607: note = Synthetic
  misc_feature 1..5607: note = Cas9 gene codon optimized for Parachlorella that includes sequences encoding an N-terminal FLAG tag, nuclear localization signal, and peptide linker and contains introns from Parachlorella
  source 1..5607: mol_type = other DNA
atgcccaagaagaagcggaaagtcggggactacaaggacgacgatgacaaactggagcctggggagaagccctataagtgtcctgagtgcgggaagagcttcagccaatctggagcactgacaaggcaccagaggacacatacacgcgacaagaagtacagcatcgggctggatatcgggaccaattctgtgggatgggccgtgattaccgacgagtataaggtgcccagcaagaagttcaaggtgctggggaacacagaccgccacagcattaagaagaacctgatcggggcgctgctgtttgattctggagagacagcagaggcaaccgtgagtgagaacagttttcagatcgaatagcacccccccgcctctgcagcagtcgcataccggctgcagtaatagcttggttcaacggcgacctgaacaagtactgtagtttctatgcatacgaactttatcgaatagaatcacgcttgggtatcgatcataccttagcgctcaatttcattggctgctacagaccatattttcctcttcacttgttgcagcgcctgaaaagaacagcaagaaggcgctacacccgccgcaagaataggatttgctacctgcaagagatcttcagcaacgagatggccaaggtggacgacagcttcttccatagactggaggagtcgttcctggtggaggaggataagaagcacgagaggcaccccatcttcggtgagaagagtttggctaccaaatctatcttttcatatcacatataccgcctgatattctgaggtggtggcttttgtctttttctttcagtatttttcttcgttgggaacctaccgcgagggcattcattgtggcggatctgtaagtgcgaccaggctgtatccaatattttttcctatcgcagggaacattgtggatgaggtggcctaccacgagaagtaccccacaatctaccacctgcgcaagaagctggtgagaatctctgcttgtcgaatgtgtccagttgtgtcttgaatcctggcaagatgttcttttcaccatccgtcctgcaaaagtgtcagaagtagcatctctcgatcgcgttgtcacttcaacgcctccgcaactccccccgttgtgaatcctgtggtcatggctcagcttttcagatctctacctgcatgttgtttgcctgtctcagtcctgcctgcacaaatcatcgcccttgtttactccttgcaatcacggattgtgtgcaggtggacagcacagataaggccgatctgaggctgatctacctggcattggcccacatgatcaagtttagggggcacttcctcatcgagggggatttgaaccccgacaacagcgatgtggacaagctgttcatccagctggtgagtggagggctggggtttgggggtggggggtggggagggaggcacggatggtgttttctcatgtccaaccgtggttcatgcaaccgaacagcagtttcacaagatggttccaacagggtgctccatttctccctgacaaaacctcgtgcggtccatctggtatagctgggttagtagggggttgtgggctgtccacagtcagtgcgaagcaggctctattgagcgtgtgctagtgtgtgctgtgctgattggcattttgttgggccgagtgttaggattagggtaaatcaccctaattaaccttacataataggactgtatgcaaatttgttttccaaaaactctacccagcgtggtcagactgcatgcactgtggagcatgcatggggctgaccctgttgatcctgctcattctgcttcctccaggtgcagacctacaaccagctgtttgaggagaaccccatcaacgcatctggggttgacgcaaaggccattctgtctgcaaggtaggtgcaggaagaagtgaatgatgcacacatggtggaatcgtgatacaagcagcagcaagtgttggaccaagacatgtgcgtgctttgctgctgccaagctggcactgcaccaggtcgtgcattgatctgcacatttgatatactgtgagagtcagacgacgtcctttcagagcctgtgtgtgattctccaggggttaacacgagtttcctttctgccagtgagtcaccctctcgctgctcgctcctggtgcaggctgagcaagtcaaggagactggagaacctgatcgcccaattgcctggagagaagaagaacgggctgttcgggaacctgatcgcattgtctctggggttgacccccaacttcaagagcaacttcgacctggcagaggacgcaaaactgcagctgagcaaggacacctacgacgatgatctggacaacctgctggcccagattggagatcagtacgcagacctgttcctggcagccaagaatctgagcgacgcaattctgctgagcgacattctgcgcgtgaacaccgagatcaccaaggcacctctgagcgcaagcatgatcaagaggtacgacgagcaccaccaagacctgacactgctgaaagcactggtgagacagcagctgcctgagaagtacaaggagatcttcttcgaccagagcaagaacgggtacgctgggtacattgatggaggagcaagccaagaggagttctacaagttcatcaagcccatcctggagaagatggacgggacagaagagttgctggtgaagctgaatcgcgaggatctgctgaggaagcagaggacattcgacaatgggagcatcccacaccagatccatctgggagagctgcacgcaattctgaggagacaagaggacttctacccgttcctgaaggacaatcgcgagaagatcgagaagatcctcacgttccgcatcccgtactatgtgggacctctggcaagggggaactctagatttgcctggatgacccgcaagagcgaggagacaattacaccctggaacttcgaggaggtggtggataaaggggcatctgcacagagcttcatcgagaggatgaccaacttcgacaagaacctgcccaacgagaaggtactgcctaagcattcactgctgtacgagtacttcaccgtgtacaacgagctgaccaaggtgaagtacgtgacagaggggatgaggaagccagcatttctgagcggagagcaaaagaaggccatcgtggatctgctgttcaagaccaaccgcaaggtgaccgtgaagcagctgaaggaggactacttcaagaagatcgagtgcttcgacagcgtggagatttctggagtggaggaccgcttcaacgcatctttggggacataccacgacctgctgaagatcatcaaggacaaggacttcctggacaacgaggagaacgaggacatcctggaggacattgtgctgacactgaccctgttcgaggatagggagatgatcgaggagcgcctgaagacatacgcacacctgtttgacgacaaggtgatgaagcagctgaagaggaggcgctatactggatggggaaggctgtcaaggaagctgattaacgggatccgcgacaagcagagcgggaagacaattctggacttcctgaagagcgacgggttcgcaaaccgcaacttcatgcagctgatccacgacgatagcctgaccttcaaggaggacatccagaaggcccaagtgtctggacaaggggatagcctgcatgagcacatcgcaaatctggctgggtcacccgcaatcaagaagggaattctgcagaccgtgaaggtggtggatgagctggtgaaggtgatgggaaggcacaaacccgagaacatcgtgatcgagatggcaagggagaaccagacaacccagaagggacagaagaactctagggagcgcatgaagcgcatcgaggagggaattaaggagctgggaagccagatcctgaaggagcatcctgtggagaacacccaactgcagaacgagaagctgtacctgtactacctgcagaacgggagggacatgtacgtggatcaagagctggacatcaaccgcctgagcgactatgacgtggaccacattgtgcctcagtcgttcctgaaggacgacagcatcgacaacaaggtgctgacaaggagcgacaagaatcgcggaaagagcgacaacgtgccttcagaagaggtggtgaagaagatgaagaactactggcgccagctgctgaacgcaaagctgattacacagcgcaagttcgacaacctgaccaaggcagagaggggaggactgtcagaactggataaggccgggttcatcaagaggcaactggtggagacacgccagatcacaaagcatgtggcccagattctggacagccgcatgaacaccaagtacgacgagaacgacaagctgatccgcgaggtgaaggtgattaccctgaagagcaagctggtgagcgactttcgcaaggacttccagttctacaaggtgcgcgagatcaacaactaccaccacgcacacgacgcctacctgaatgcagttgtgggaacagccctgatcaagaagtaccccaagctggagagcgagttcgtgtatggggactacaaggtgtacgacgtgcgcaagatgatcgccaagtctgagcaagagatcgggaaggcaaccgccaagtacttcttctacagcaacatcatgaacttcttcaagaccgagatcaccctggccaatggggagattaggaagagacccctgatcgagaccaacggagagactggagagatcgtgtgggataaggggagggactttgcaacagtgcgcaaagtgctgagcatgcctcaagtgaacatcgtgaagaagaccgaggtgcagactgggggattctcaaaggagagcattctgcccaagcgcaacagcgataagctgattgcacgcaagaaggactgggaccccaagaagtatggggggtttgatagccccaccgtggcatattctgtgttggttgtggccaaggtggagaaggggaagagcaagaagctgaagagcgtgaaggagctgctggggatcaccattatggagaggagcagcttcgagaagaaccccatcgacttcctggaggcaaaggggtataaggaggtgaagaaggacctgatcatcaagctgcccaagtacagcctgttcgagctggagaatgggaggaagaggatgctggcatctgctggagaactgcagaaggggaatgagttggcactgcctagcaagtacgtgaacttcctgtacctggccagccactacgagaagctgaagggatcacccgaggacaatgagcagaagcagctgtttgtggagcagcacaagcactacctggacgagatcatcgagcagatcagcgagttcagcaagcgcgtgattctggcagacgcaaacctggataaggtgctgagcgcctacaacaagcaccgcgataagcccattcgcgagcaagcagagaacatcatccacctgttcaccctgaccaacctgggagcacctgcagcattcaagtacttcgacaccaccatcgaccgcaagaggtacacaagcaccaaggaagtgctggacgcaaccctgattcaccagagcattactgggctgtacgagacacgcatcgacctgtcacaactgggaggggactga

SEQ ID NO: 77 - DNA, 588 bp, Parachlorella sp.
  misc_feature 1..588: note = RPS17 promoter
  source 1..588: mol_type = unassigned DNA
caacacctagttggtaaataccgttgctgatattgctctgtaccagtaaaagagggctgcgatgagcgtttttagtgcacttcttcaacacggaatatttttcacaaattggtatgagaaccaattttgcaaaatgttcgccctgtaaagtatcgctctgggacgatcagcttgacgtaattgtaggcgaaaagggcgttcaaagtgcagctttatgtatgaacgtcataaaatataaagcatagcacaatcactgatagaaaatatttgtgcgcattaaaactctcacttctgttgcggatacaacgacggaaatgagaagcttgtgtaagaagcaattcaagttttcattttgtcatctaaggtgtgatcctccgatattcattaccgaatgctgatctgagttggaaagatggcaatatttagctgtgcacactttgacctccaggccttggcgggaatttagtattctagctttcctattggaacgataggccagccaagtctccagcttgtatacgctacaccagcagacatgctctcaatttagctgacagtgtcttcatatttgtattatctgttgtgtct

SEQ ID NO: 78 - DNA, 455 bp, Parachlorella sp.
  misc_feature 1..455: note = RPS17 terminator
  source 1..455: mol_type = unassigned DNA
ggtgcgaatagtgcttcagtaaaaaagtagcaacttggtgcaatatcgtcagggtcgtgtggtctgctcgccagcaagttttttggcacaggagagcgctttttccgagtaccgccaaagttcaagcatgtgctgtgattcgctgttgcctcttatgataattgctcaaagtttccaagcattctatgtccaccctgcaccactaagttgtatggtgcttattctgcaggggatgattcatggtgcctaaaaattttgtgctgctgtcgcgtctgttttctgtcgcagtttagtgaatgtaactccaaataccaaacttttcatcacaatcatattgatgcctttgtaagtgaattacagcgttttttgccataaaaagaagtaccgtgacattggggtcgtcataacaagaagctttatgaacaagcagcttgatctacgagacttatacataa

SEQ ID NO: 79 - DNA, 2691 bp, synthetic construct
  misc_feature 1..2691: note = Synthetic
  misc_feature 1..2691: note = Basticidin resistance gene codon optimized for Parachlorella, containing Parachlorella introns
  source 1..2691: mol_type = other DNA
atggcaaaacctctctcccaggaagagtctaccctgattgaaagggcaacagcaaccatcaacagcattcccattagcgaggactactctgttgtgagttctgagaagctgattgttgtttaacttctttgaaagctttatcgaagattctgcaagcgatgaacattgcttgtcaagaccgagagctgcatgcccacttgacatccagctttgaacggctcttcatgtttgatttgtttctgattgtaggcatctgcagcattgagctctgatggaaggatctttacaggagtgagtgcagcgtcagctgtggcagttgttggctttcgtctcagtcagtagtttgctgggattgattatggagggcacagttgcaattttgagttgcacgttgcgacaagcgtgttgacaaagcgtggtcaagccggccagtcttgccggtggcgggtggcttggtctaacttccgctctacggcaatcgttttgttcatggttacggggctggcgtgccagaaagtcctggtcagccaccctcgcttcaaagccgtagcccaacaactttgcgaatatgttcgatttgcaggtgaacgtgtaccactttacaggaggaccttgtgcagaattggtggtgttaggtacagctctgcgtgcaacaggttgcaagatgcagcgcaggtcttccctggtcaaacgatgtatgcagagttgagaggcacttgagctgggtgaatggcgtgggctcgtaggtagtgtgcagggcaggaagggcagccaattttggagttgtggtccggtgtcgttgcttcgagccttattaggactcttgctcatcaaagcgttagttgtgaataagttgatctgaaaggatgttatgtacagcaagcagcagcagttaagagtctggggagtagctgcacagggcgaggtgtcaagatgggaagggtcctgcctccttatgtgtttttccctgtaggggaggaagcctcttatgggcaatggttgggcatattttccagccagcccttctttctataggggccagggtgggcccagctcgtcttggcttccaccaccaggagagtgagggcattgaagggccataaatagtcctcccatctacgtgcaccagagggtgtcgtctaggctgtgcatgccacgaggggaaggagccaagaatgagtgtatgggttgttttcatgtttaggctgggataaaactgttttcaattgcgcctgccgggtgaaaaccacagcagcatcagcaagcttggagaaggccagcccgcccagcacaggctcacgttcccactcaggcggtcagtcgggcgggggtgtgagtcaggcaggcgagggtgtctgtgcctgacatcagcacctctgcttagccactgcagcccctggagcagggtagggcgtcatttgcagcaatcacctgctgcctcacacgtcgcagcttggaatttcaacgaccatcagcgctggggttgttgagggatcatagcagattttggtgcagcctggttgtcatgctctttgtggaatggcctctatgttcgagcaattcgttggatgttgaggtgcttggggacagagagtcgaatgatgggccagggtcaaacatgcgagcgtttggctgagtcagcggtttttgctggtcactttttcttttgtttcttatttaggtttgatggatgtgttttgtgctgctgccctgaagctgcagcagcgtgtctgccctgcgctactgcgggcaccaaggctatgtgctggtgcactcggctgcgctgcacctgtgcacctcgcactccgtccagcctccatgcagcacacgtactcacggtgtcctcctgacctgtcgtacgctattccaaacttgctcttttgctgccgctgctctcgtacacaattgctgttgattatcgatatctaatcgagcgcctgctgactgaactccgcaggtacagcagctgcagcagcagcaggaaatttgacatgcattgtggcaataggtgggtgggctctgaaggaggaggagggagcgggtgattaaacagggcctgcatgaagaggagcaggggctgcgtggacagcagggggaaggtgcagaagggagggtcaagcggggttcaggtggctgtgggtttctgcacgagcagtgaaagaagctgtatccttccacctgcttccactggcgaaaggttgaaaacaggatgtcgcagctggaaagatgttgcgctgtcaagtgcaagccatggttgagggtatgcctgtgtgcatgtgcttcttaaagttactcctgttctatggttctgggtgcttgttgtttgtggtgcagggaacgagaatagggggattttgtcaccttgcggaagatgtagacaggtgttgttggatctgcatcccgggattaaggtgagggggcatgtaagcaatggcaggcaattcaagaacgaatcattgctgcaaatgctgggatggtatgcagctgaggtatctattgccttgtattttgtctcgcattgcatcggtggtgcgttctgtggcctgaggcacagttcttgctgtttgataagggttcgactgagttgtcgtgtgtgctgtgctgcaggcaatcgtgaaggattcagatgggcagcctacagcagtgggaattagagaactgctgccttctgggtatgtgtgggaaggataa

SEQ ID NO: 80 - DNA, 530 bp, Parachlorella sp.; identical to SEQ ID NO: 7
  misc_feature 1..530: note = RPS4 promoter
  source 1..530: mol_type = unassigned DNA

SEQ ID NO: 81 - DNA, 546 bp, Parachlorella sp.; identical to SEQ ID NO: 8
  misc_feature 1..546: note = RPS4 terminator
  source 1..546: mol_type = unassigned DNA

SEQ ID NO: 82 - DNA, 702 bp, synthetic construct
  misc_feature 1..702: note = Synthetic
  misc_feature 1..702: note = Encodes TurboGFP
  source 1..702: mol_type = other DNA
atgttggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctgcagaacgggggccccatgttcgccttccgccgcgtggaggaggatcacagcaacaccgagctgggcatcgtggagtaccagcacgccttcaagaccccggatgcagatgccggtgaagaataa

SEQ ID NO: 83 - DNA, 572 bp, Parachlorella sp.
  misc_feature 1..572: note = ACP1 promoter
  source 1..572: mol_type = unassigned DNA
agtttgcatagttaagtatgctggctattgcagtaccttatatgcaaacaagtgctcaatctgtttcatcattgtctgtgggcaaattgcctgccaatattctccagttattgcctgttgtttcaaatgattgaaattggaagttgtattgctctacatttttgacttgtgattttttcatttgttgatatctgacaactgtgaactgcactgaacttgctgtgcttataaatgcatttttttgttttgggccacgttgattccttgtgatactttcctgctatcaaaccaaaaatatactctcatgactgacgtgcaacaaatgcatggaagctttcaacgttacgacagctgcttgccccccatcagctattctacatgtgtaacctaccttgcatggccaccacaacgctactgcatgcaagatctggcgcaactggatgtcccaatagtagaagtatccggattatctccgagagttttacatatgtaatcgacgccatttctgtcatcaactataaatccattgctcctgcatttctggcactgacattctaccacaagcaatacca

SEQ ID NO: 84 - DNA, 869 bp, Parachlorella sp.
  misc_feature 1..869: note = ACP1 terminator
  source 1..869: mol_type = unassigned DNA
gcagcagcttgttatgccttccccatgggcatcagcatgctgcaagctgtctagatatccagctttcagtggaggttgagcgagggtcagcagcggttccctggcgatggcggtcagcttttctggaagccttcactaggactgcgcccagcgcatgtgacgccaatcgaacttgtgtgcaaggccaaattttgtgaccctgtgctgcacttcatgtattcaagaattgagaagaaatttcattgctgcccttctttcactttaatttccatccctggatccacctcccaccattgtggttgatgggtaggggttttgggtaggtgcagttcgttgtgcacgttgacatgtgtaacggtgagcaaaggaattgctgggcaagtagctattgcagcttaagggcatggtgaaacacttgtgctgtatttacagaggaagccagacaggtaaggagtgtgtggcagcttggaacaggagggctggtcgcaacaagtatgcatatcccatgattgttgacataagagcagcaggtgcatattgccagcctttgtgaaagtggattgaaaatcgattagttggtgtgatagctgaggctaggcactgccaacctgcagtgaaatgaggctccaagaccgggtaataatacaggcaatcgaatccagttgaaattacggcgattaaatccaagcgagcgttgtaagaacatctgcacctgtctgaagtagtgagcggataatgagcattgcttgccttctatcactatacctgacagttacgtgtcacacactctcaagcacaacacacagcggcaaagttacttgctaaacctcacagtcaagctgaaaataaaggctaaattacgtgagacc

SEQ ID NO: 85 - DNA, 2879 bp, Tetraselmis sp.
  misc_feature 1..2879: note = SGI1 gene, T-5172
  source 1..2879: mol_type = unassigned DNA
atgagctgcacagtggcgtcctttccgcccgcggcgggggggcagggctcgcctgccacgcccgtcccataccaggacctgctcgtgaagcggcaagaccagtggagcaacttcccggccgggctgcgcgtgctggtggcagacaacgaccccgcctcgctgcagcaagtggagaagatgctaaagaagtgtagctaccaaggtgaggctgcgctgcccctcgaaaggcggccggccccccaccccagctggccacttgcgcagtgcgcgtgttatcttgataatccgaaaacgcgtttttttgcctcgttttatcccgctagtcgtacaatccggtcggggcccaggcaggcaccggcggccacgcgcagacccacacaccagctcaccggttgctgctacactgatgcatgctttacgcccggtcccaacagtaacactgtgcagctcggggaagaactccctggagatcctccgcaagcggcgagaggaattcgacctcgtgctggcggacgcaaatctaccaggttttccccctcaccctgtctgctcgagggggtacaccggtactactaaccgcgacaattccgcccgctcaccgccgccttcccacgccacaagcgacggaactgattgtattcctgctcccggtctttcgcctgcagacatagatggcttcaagcttcttcacgtctgccacaccgagctgtcgctccccgtcgtccgtaagagcctgctgcccgctcgacatccacctcgcacttcaatttgctattcagtcatcgtaccgtgccctgccctgcacttccctgccctgaggtatcccagtcgcatcccggccttccccccccccccccccccccttcccccaaacgtagtgatgtccggcaccagcgacacacagctggtaatgcgcggcgtgatggatggcgcgcgggactttctgatcaagcccctgcgcgtggaggagctcaaggtgctctggcagcaccttgtgcgcttcacttcggagatcaccaagacagacgcgcagctaaatgttgtcaaggtggagctggacggcggaaggcctgcgggcgaagtctctacgagtcagaacggctcggtatgccacagcagcccatttgaagcagcccgtttcttgtagggcgcgaattatgcctcggcgcgttttcctccctcctcgcgctgactccttgtgtgccccacacagcaatgcaccgagcgcgagggcgaaggcaattcatcaaagaagcagcgcatgaattggagcgatgaaatgcatcagcagtttgtcaatgccgttaaccagcttggcatcgacagtgcgatcccccccccccccccccccccccctcttcctgcgactgctctccgctctccataagagtaattgcgctcgcccacatgagctcgtgccgaccctgctgtcctgcgctgctcccggggtccgcagaggccgtacccaagcggattctagacctcatgagcgtggagggcctcacacgagaaaacgtagccagccacttgcaggtcccccccccaccccctttctcggcatgttgggcgagtgactggtcaacccctgcaatgatggctgaaccacagagcacttctgagatgcatgttcttctgcagctgctgacctgtgcaccccctccgccacatcggattcccccctccctgtcatcctcctcccatgccccgcacgcgtgcagaaataccgcatctacttgaagcgcatggccaaccaccaggaaaatgggaagcaagcggtgatgagcacggacacgatcgcccgcgctgaagcggcgtaccagggaggcatgccccaggggcagcagatgatgcagcaggagcacagcggccaggcggtgcagtacagccagccgcatgcccccggcggcctgcaccagcaagccatgcccgcccagatgcacatgggcatgatgccggccggcccgcagcccggcagcatgcagatggcgccgcaccacgtcatgcaaatgcccaatgggcaggtgatggtgatgcagcagatggggccgcgcccggggatgccacccgggatgccgcagcagatgatggccagctcgcagcagatgggcatgcttcagcccggcatgccggcgggtcagatgttgcactttcagcacccgcagcaagtgcaccagcatccgccaagctcagggcctatgcacgcaggtgcgctacgcccctcaatgagtccatccccccccccccccccccccaacccgcccaagaaaaacatgcagtaaatacgctctgttttcgcatcaacatttgattttagggacaaagagaatgacctgtgcatgcagggttggaaaacttaacattcacgcaccatacaagtacatcatagcctttgtatgttgcaatattagaggccactttcagagtttcagtggctacctctattggcaattctgccgcgtctacgtgcccatgtgttgttccatttaattggccttgcggctcgtcgcgcggcgtgaagcctaccatggtgcatttgttttcctctttgatgcttattgacctggtttctcccctgtgtccatgtagaatgtgtttttagtgtgtgcatatcagtcgatggggtgttgtcttttgattgctgctgcactctctttgtggctgatgtagatctgtctgacgcagggggagagatgatagacccaggcagcatgcagcggttgcatcagcagccacactacatcggccccaacgggcagcacatgccagcgcctgcgatggggatgccgtctggcacagtccagcacatggagtacgcctacagccagcccatgcagatggctggctggccggtccaaggccaacccggcaaccaagcctag

SEQ ID NO: 86 - DNA, 1341 bp, Tetraselmis sp.
  misc_feature 1..1341: note = SGI1 T-5172 coding sequence
  source 1..1341: mol_type = unassigned DNA
atgagctgcacagtggcgtcctttccgcccgcggcgggggggcagggctcgcctgccacgcccgtcccataccaggacctgctcgtgaagcggcaagaccagtggagcaacttcccggccgggctgcgcgtgctggtggcagacaacgaccccgcctcgctgcagcaagtggagaagatgctaaagaagtgtagctaccaagtaacactgtgcagctcggggaagaactccctggagatcctccgcaagcggcgagaggaattcgacctcgtgctggcggacgcaaatctaccagacatagatggcttcaagcttcttcacgtctgccacaccgagctgtcgctccccgtcgtcctgatgtccggcaccagcgacacacagctggtaatgcgcggcgtgatggatggcgcgcgggactttctgatcaagcccctgcgcgtggaggagctcaaggtgctctggcagcaccttgtgcgcttcacttcggagatcaccaagacagacgcgcagctaaatgttgtcaaggtggagctggacggcggaaggcctgcgggcgaagtctctacgagtcagaacggctcgcaatgcaccgagcgcgagggcgaaggcaattcatcaaagaagcagcgcatgaattggagcgatgaaatgcatcagcagtttgtcaatgccgttaaccagcttggcatcgacaaggccgtacccaagcggattctagacctcatgagcgtggagggcctcacacgagaaaacgtagccagccacttgcagaaataccgcatctacttgaagcgcatggccaaccaccaggaaaatgggaagcaagcggtgatgagcacggacacgatcgcccgcgctgaagcggcgtaccagggaggcatgccccaggggcagcagatgatgcagcaggagcacagcggccaggcggtgcagtacagccagccgcatgcccccggcggcctgcaccagcaagccatgcccgcccagatgcacatgggcatgatgccggccggcccgcagcccggcagcatgcagatggcgccgcaccacgtcatgcaaatgcccaatgggcaggtgatggtgatgcagcagatggggccgcgcccggggatgccacccgggatgccgcagcagatgatggccagctcgcagcagatgggcatgcttcagcccggcatgccggcgggtcagatgttgcactttcagcacccgcagcaagtgcaccagcatccgccaagctcagggcctatgcacgcagtccagcacatggagtacgcctacagccagcccatgcagatggctggctggccggtccaaggccaacccggcaaccaagcctag

SEQ ID NO: 87 - DNA, 2909 bp, Tetraselmis sp.
  misc_feature 1..2909: note = SGI1 T-5185 gene
  source 1..2909: mol_type = unassigned DNA
atgactcctaccccacccatgagctgcacagtggcgtcctttccgcccgcggcgggggggcagggctcgcctgccacgcccgtcccataccaggacctgctcgtgaagcggcaagaccagtggagcaacttcccggccgggctgcgcgtgctggtggcagacaacgaccccgcctcgctgcagcaagtggagaagatgctaaagaagtgtagctaccaaggtgaggctgcgctgcccctcgaaaggcggccggccccccaccccagctggccacgtgcgcagtgcgcgtgttatcttgataatccgaaaacgcgtttttttgcctcgttttatcccgcaagtcgtacaatccggtcggggcccaggcaggcaccggcggccacgcgcagacccacacaccagctcaccggttgctgctacactgatgcatgctttacgcccggtccccaacagtaacactgtgcagctcggggaagaactccctggagatcctccgcaagcggcgagaggaattcgacctcgtgctggcggacgcaaatctaccaggttttccccctcaccctgtctgctcgagggggtacaccggtactactaaccgcgacaattccgcccgctcaccgccgccttcccacgccacaagcgacggaactgattgtattcctgctcccggtctttcgcctgcagacatagatggcttcaagcttcttcacgtctgccacaccgagctgtcgctccccgtcgtccgtaagagcctgctgcccgctcgacatccacctcgcacttcaatttgctattcagtcatcgtaccgtgccctgccctgcacttccctgccctgaggtatcccagtcgcatcccggccacccccccccccccccccccccccccttcccccaaacgtagtgatgtccggcaccagcgacacacagctggtaatgcgcggcgtgatggatggcgcgcgggactttctgatcaagcccctgcgcgtggaggagctcaaggtgctctggcagcaccttgtgcgcttcacttcggagatcaccaagacagacgcgcagctaaatgttgtcaaggtggagctggacggcggaaggcctgcgggcgaagtctctacgagtcagaacggctcggtatgccacagcagcccatttgaagcagcccatttcttgtagggcgcgaattatgcctcggcgcgttttcctcccgcctcgcgcgctgactccttgtgtgccccacacagcaatgcaccgagcgcgagggcgaaggcaattcatcaaagaagcagcgcatgaattggagcgatgaaatgcatcagcagtttgtcaatgccgttaaccagcttggcatcgacagtgcgatccctcccctccccccccgcccccccacccacccccctcttcctgcgactgcgctccactttccttaagagtaattgagctcgcccacatgagctcgtgccgaccctgctgtcctgcgctgctcccggggtccgcagaggccgtacccaagcggattctagacctcatgagcgtggagggcctcacacgagaaaacgtagccagccacttgcaggtcccccccccccccccccctttctcggcatgttgggcgagtgactggtcaacccctgcaatgatggctgaaccacagagcacttctgagatgcatgttctttctgcagctgctgacctatgcaccccctccgtcacatcggattctcccctccctgtcatcctcctcccatgccccgcacgcgtgcagaaataccgcatctacttgaagcgcatggccaaccaccaggaaaatgggaagcaagcggtgatgagcacggacacgatcgcccgcgctgaagcggcgtaccagggaggcatgccccaggggcagcagatgatgcagcaggagcacagcggccaggcggtgcagtacagccagccgcatgcccccggcggcctgcaccagcaagccatgcccgcccagatgcacatgggcatgatgccggccggcccgcagcccggcagcatgcagatggcgccgcaccacgtcatgcaaatgcccaatgggcaggtgatggtgatgcagcagatggggccgcgcccggggatgccgcccgggatgccgcagcagatgatggccagctcgcagcagatgggcatgcttcagcccggcatgccggcgggtcagatgttgcactttcagcacccgcagcaagtgcaccagcatccgccaagctcagggcctatgcacgcaggtgcgctacgcccctcaatgagtccatgccccccccaacccgcccaagaaaaacatgcagtaaatacacactgttttcgcatcaacatttgattttagggacaaagagaatgacctgtgcatgcagggttggaaaacttaacattcacgcaccatacaagtacatcatagcctttgtatgttgcaatattagaggccactttcagagtttcagtggctacctctattggcaattctgccgcgtctacgtgcccacgtgttgttgcatttaattggccttgcggctcgtcgcgcggcgtgaagcctaccatggtgcatttgctttcctctttgatgcttattgacctggtttctcccctgcgtccatgtagaatgtgtttttagtgtgtgcacatcagtcgatggggtgttgtcttttgattgctgctgcactctctttgtggctgatgtagatctgtctgacgcagggggagagatgatagacccaggcagcatgcagcggttgcatcagcagccgcactacatcggccccaacgggcagcacatgccagcgcctgcgatggggatgccgtctggcacagtccagcacatggagtacgcctacagccagcccatgcagatggctggctggccggtccaaggccaacctggcaaccaagcctag

SEQ ID NO: 88 - DNA, 1473 bp, Tetraselmis sp.
  misc_feature 1..1473: note = SGI1 T-5185 coding sequence
  source 1..1473: mol_type = unassigned DNA
atgactcctaccccacccatgagctgcacagtggcgtcctttccgcccgcggcgggggggcagggctcgcctgccacgcccgtcccataccaggacctgctcgtgaagcggcaagaccagtggagcaacttcccggccgggctgcgcgtgctggtggcagacaacgaccccgcctcgctgcagcaagtggagaagatgctaaagaagtgtagctaccaagtaacactgtgcagctcggggaagaactccctggagatcctccgcaagcggcgagaggaattcgacctcgtgctggcggacgcaaatctaccagacatagatggcttcaagcttcttcacgtctgccacaccgagctgtcgctccccgtcgtcctgatgtccggcaccagcgacacacagctggtaatgcgcggcgtgatggatggcgcgcgggactttctgatcaagcccctgcgcgtggaggagctcaaggtgctctggcagcaccttgtgcgcttcacttcggagatcaccaagacagacgcgcagctaaatgttgtcaaggtggagctggacggcggaaggcctgcgggcgaagtctctacgagtcagaacggctcgcaatgcaccgagcgcgagggcgaaggcaattcatcaaagaagcagcgcatgaattggagcgatgaaatgcatcagcagtttgtcaatgccgttaaccagcttggcatcgacaaggccgtacccaagcggattctagacctcatgagcgtggagggcctcacacgagaaaacgtagccagccacttgcagaaataccgcatctacttgaagcgcatggccaaccaccaggaaaatgggaagcaagcggtgatgagcacggacacgatcgcccgcgctgaagcggcgtaccagggaggcatgccccaggggcagcagatgatgcagcaggagcacagcggccaggcggtgcagtacagccagccgcatgcccccggcggcctgcaccagcaagccatgcccgcccagatgcacatgggcatgatgccggccggcccgcagcccggcagcatgcagatggcgccgcaccacgtcatgcaaatgcccaatgggcaggtgatggtgatgcagcagatggggccgcgcccggggatgccgcccgggatgccgcagcagatgatggccagctcgcagcagatgggcatgcttcagcccggcatgccggcgggtcagatgttgcactttcagcacccgcagcaagtgcaccagcatccgccaagctcagggcctatgcacgcagggggagagatgatagacccaggcagcatgcagcggttgcatcagcagccgcactacatcggccccaacgggcagcacatgccagcgcctgcgatggggatgccgtctggcacagtccagcacatggagtacgcctacagccagcccatgcagatggctggctggccggtccaaggccaacctggcaaccaagcctag

SEQ ID NO: 89 - DNA, 3005 bp, Tetraselmis sp.
  misc_feature 1..3005: note = SGI1 T-5230 gene
  source 1..3005: mol_type = unassigned DNA
atgagctgcacagtggcgtcgtttccgccagcggcggggggggcagggcgcgcctgccgcgcccgtgccataccacgacctgctcgtgaagcggcaagaccagtggagcaacttcccggccgggctgcgcgtgctggtggcagacaacgaccccgcctcgctacagcaagtggagaagatgctaaagaagtgtagctaccaaggtgaggctgctctgcccctcgaagggcggccggcaccccaccccagctagacacgttcgcagtgcgcgtgttatcttgataacccgaaaacgcgtttttttgcctcgttttatcccgctagtcgtacataccggtcggggcccaggcaggcaccggcggccacgcgcagacccacacaccagctcacccgttgctgctacactgatgcatgctttacgcccggtccccaacagtaacactgtgcagctcgggcaagaattccctggagatcctccgcaagcggcgagaggaattcgacctcgtgctggcggacgcaaatctaccaggttttccccctcaccctgtgtgctcgagggggtacaccgatactacaaaccgcgacaattccgcccgctcaccgccgccttcccacgccacaagcgacgaaactgattgtatttctgctcccggtcttttgcctgcagacatagatggcttcaagcttctgcacgtctgccacaccgagctgtccctccccgtcgtccgtgagagcctgctgccacccgctcgacatccagctcgcacttcaatctgccattcacttataccgtgccctgccctgcacttccctgccctgaggtatcccattcgtatcccggcatgggttctgaaagcgctcaccgcgcccgccccccccccccccccccccccccaacgtagtgatgtccggcaccagcgacacgcagctggtaatgcgcggcgtgatggatggcgcgcgtgactttctgatcaagcccctgcgcgtggaggagctcaaggtgctctggcagcaccttgtgcgcttcacttcggagatcaccaagacagacgcgcagctaaatgttgtcaaggtggagctggacagcggaaggcctgcgggagaagtctctacgagtcagaacggctcggtatgccacagcatctcatttgaagccgcgtcttccttgtagggcgcgaatcatgcctcggcgcgtctccatctcgcctcgcgctgactttctgtgtgccccaaacagcaatgcgccgagcgtgagggcgaaggcaattcatcaaagaagcagcgcatgaattggagcgatgaaatgcatcagcagtttgtcaatgccgttaaccagcttggcatcgacagtgcgacccccccccctcttcctgcgactgcgctccactctccttaagagtaattgcactcccccacataagctcgttccgaccctgctgtcctgcgctgctccgggggtccgcagaggccgtacccaagcggattctagacctcatgagcgtggagggcctcacacgagaaaacgtagccagccacttgcaggtcccccccccccccccccccccttctcggcatgttgggcgagtgactggtcaaccctgcaataatggctgaaccacagagcacttcttagatgcatgtttttcctgcagctgctgacctatgctccccccccccccccccccccccccgtcacatgcgattccctccccccccctccctgcgctccgtcacatcggattccccccttcctgtcatcctcctgcaatgccccgcgcgcgtgcagaaataccgcatctacttgaagcgcatggccaaccaccaggaaaatgggaagcaagcggtgatgagcacggacacgatcgcccgcgctgaagcggcgtaccagggaggcatgccccaggggcagcagatgatgcagcaggaacacagcggccaggcggtgcagtacagccagccgcatgcccccagcggcctgcaccagcaagccatgcccgcccagatgcacatgggcatgatgccggccggcccgcagcccggcagcatgcagatggcgccgcaccacgtcatgcaaatgcccaacgggcaggtgatggtgatgcagcagatggggccgcgcccggggatgccgcccgggatgccgcagcagatgatggccagctcgcagcagatgggcatgcttcagcccggcatgccggcgggtcagatgttgcactttcagcacccgcagcaagtgcaccagcatccgccaagctcagggcctatgcacgcaggtgcgctacgcccctcaatgagtccatgccccccccccccccccccccacccccccccccccagaatgcagtaaacacactctgtttccgcttcgttatttggttttagagaataaccagtgcatgcaggattggaatacttatcatttacgcaccatgcaactacaccacaccctttgcatgtcgcaatattagaggccacttttagagtttcagggactatctctattggcaattctgccgcgtctacgtgcccatgtgctgttccagtaattggcctcgccgctcgtcacatggcatgaagcccaccatggtgcatttgctgtactctgaagcttcttgacctggtttctcccatgtgtgtgtgtgtgtgtgtgtgtgtgtgttgttgtggggggggggggggggcagaacatttttcaggcacgaacccatgtagagtgtggttttagtttgtacatatcagtgaatgaggtgttgtcttttgattgttgctgcactctctttgtggctgatgtagatctgtctgacgcagggggagagatgatagacccaggcagcatgcagcggttgcatcagcagccgcactacatcgtccccaacgcgcagcacatgccagcgcctgcaatggggatgccgcctggcgcagtccagcacatggagtacgcctacagccagcccatgcagatggctggctggccggtccaaggccaacctggcagtcaggcctag

SEQ ID NO: 90 - DNA, 1725 bp, Tetraselmis sp.
  misc_feature 1..1725: note = SGI1 T-5230 coding sequence
  source 1..1725: mol_type = unassigned DNA
atgaccatgccactcggcggtggtctgtgcatgaaggatcgcatccatggggatgagaggtaccgttccaaggccaaacgccaagtaaatacgatatttgcgttcacccagcgcaacacatggcgcggacgcttccggctatgctcttataggacaactgaattactgggcggaagcaaaacgacggagccgggaaggggaaccttcgtgctgcagattttcatgtgtgttaagaacgcctccatcgacgacggctcacgccatatttccacgagccgggggctggagtccgtcctgaagcggcggggggggcagggcgcgcctgccgcgcccgtgccataccacgacctgctcgtgaagcggcaagaccagtggagcaacttcccggccgggctgcgcgtgctggtggcagacaacgaccccgcctcgctacagcaagtggagaagatgctaaagaagtgtagctaccaagtaacactgtgcagctcgggcaagaattccctggagatcctccgcaagcggcgagaggaattcgacctcgtgctggcggacgcaaatctaccagacatagatggcttcaagcttctgcacgtctgccacaccgagctgtccctccccgtcgtcctgatgtccggcaccagcgacacgcagctggtaatgcgcggcgtgatggatggcgcgcgtgactttctgatcaagcccctgcgcgtggaggagctcaaggtgctctggcagcaccttgtgcgcttcacttcggagatcaccaagacagacgcgcagctaaatgttgtcaaggtggagctggacagcggaaggcctgcgggagaagtctctacgagtcagaacggctcgcaatgcgccgagcgtgagggcgaaggcaattcatcaaagaagcagcgcatgaattggagcgatgaaatgcatcagcagtttgtcaatgccgttaaccagcttggcatcgacaaggccgtacccaagcggattctagacctcatgagcgtggagggcctcacacgagaaaacgtagccagccacttgcagaaataccgcatctacttgaagcgcatggccaaccaccaggaaaatgggaagcaagcggtgatgagcacggacacgatcgcccgcgctgaagcggcgtaccagggaggcatgccccaggggcagcagatgatgcagcaggaacacagcggccaggcggtgcagtacagccagccgcatgcccccagcggcctgcaccagcaagccatgcccgcccagatgcacatgggcatgatgccggccggcccgcagcccggcagcatgcagatggcgccgcaccacgtcatgcaaatgcccaacgggcaggtgatggtgatgcagcagatggggccgcgcccggggatgccgcccgggatgccgcagcagatgatggccagctcgcagcagatgggcatgcttcagcccggcatgccggcgggtcagatgttgcactttcagcacccgcagcaagtgcaccagcatccgccaagctcagggcctatgcacgcagggggagagatgatagacccaggcagcatgcagcggttgcatcagcagccgcactacatcgtccccaacgcgcagcacatgccagcgcctgcaatggggatgccgcctggcgcagtccagcacatggagtacgcctacagccagcccatgcagatggctggctggccggtccaaggccaacctggcagtcaggcctag

SEQ ID NO: 91 - DNA, 20 bp, Tetraselmis sp.
  misc_feature 1..20: note = SGI1 MA1 target sequence for Cas9
  source 1..20: mol_type = unassigned DNA
gcaccttgtgcgcttcactt

SEQ ID NO: 92 - DNA, 19 bp, Tetraselmis sp.
  misc_feature 1..19: note = SGI1 JC2 target sequence for Cas9
  source 1..19: mol_type = unassigned DNA
cagctaaatgttgtcaagg

SEQ ID NO: 93 - DNA, 1941 bp, synthetic construct
  misc_feature 1..1941: note = Synthetic
  misc_feature 1..1941: note = Nourseothricin expression cassette
  source 1..1941: mol_type = other DNA
ggcggcatgtctgcgcaagccctgctgctaactggctcccgcgactgaattcggaatgtatgcgtggttattgcgtttcgtgtgcatatacttccgtgatactttcgccaactccatcagcagtagtttatggacaggcggcggccgccgcccgccgatgacatcgtagtcgctcgtcgtcagggaagtgccgtcaggagcgtggtggcggcggcgcggcggctcgcgcaccggcggccggtgcggaaaggtgagcggaggcgcccgtgacgtccccctttcgcccaccaccctctacgctcaacccacaggtgattcagcacgccctccgcccccgccctccaggcaaccagtcacgctagtccgccccagcgtatcgccggcccttcccgcgctggctttagcggcaggctcgccgcctgatccggcgcgccctcgccgccgccaccgagaatcttcgcagcagccgcaccgccgggaagtgtactgggcgaggggcagcaccgtgtcgcagcaaatgtgcgaggcgcgcaggaaacgcaggcatgccgctggaatccacgcggacaatggaggggaaagggggggattcgttttggtggcccccagcgtagatggggcagggtgaccaggtctccttttttccttcaactgcccagattcgtccgaggttgggcattccttgcattacgggaaataccgcgcggctctgtaatgccggttgcgatttacgccttgtacttcaaattattgtttttcggggaaacccaccaggctgggttcgctagcgaagcgtccaagcctgactgagtagctgcctgcctggtcaccacttgaggtccaccacaccgcagtcacttgccctgccgccatggcggttctgagtgctgccatctctgcctctgcagctgccaggcaggctcgccatctgcctgctgtacgccagcacatctgacctccccccgcccccctgtcttcctctctcataggaacatcctctaggacgaaaagactttgagacagctctacctgtcgaggaaaaatgaccacactcgacgacaccgcatacagataccgcacaagcgttcctggcgacgcagaggcaattgaggcattagatggcagcttcaccaccgacaccgtgtttagagtgacagcaaccggcgacggctttacacttagagaggttccagtggacccgccattgacaaaggtgtttccagacgacgagagcgacgatgagagcgatgctggtgaagatggcgacccagatagccgcacatttgtggcatacggcgatgatggcgaccttgctgggtttgtggtggtgagctactctggctggaatagacgcttgaccgtggaggatatcgaggttgcaccagagcatagaggccatggagtgggtagagcactgatgggtctggcaaccgagtttgcaagggagagaggtgctggtcatctgtggttggaggtgacaaacgtgaacgcaccagcgatccacgcatacagaagaatgggcttcacgctgtgcggcctggatacagcactgtatgatggcacagcgagcgatggtgagcaagcactgtacatgagcatgccatgcccgtaagcgctgtgcgcctagctttgcctttgcaccaacgatgacggcgatgctctgcatgtaccccgtgcgggtgtatgtaaatccatgtgccggagagcacttcgctccttcatcacactttttcgccatccgccattccccgccccagcctgaccaacctctatgccccaccacccgggactggcatgcctgaccctttcacatgcgcggtcggccttgggtcttctctctacacaagcggcgacttgctcgccaaagaggataggtcaaaggtcagtgaacatgttctctatatcacgctgtcggcgggtgttccactcggtctgtcgaaa

SEQ ID NO: 94 - DNA, 1042 bp, Tetraselmis sp.
  misc_feature 1..1042: note = RB40 promoter
  source 1..1042: mol_type = unassigned DNA
ggcggcatgtctgcgcaagccctgctgctaactggttcccggggctgaattcggaatgtatgcgtggttattgcgtttcctgtgcatatacttccgtgatactttcgccaactccatcagcagtagtttatggacaggcggcggccgccgcccgccgacgacatcgtagtcgctcgtcgtcagggaagtgccgtcaggagcgtggtggcggcggcgcggcggctcgcgccccggcggccggtgcggaaaggtgagcggaggcgcccgtgacgtccccctttcgcccaccaccctctacgctcaacccacaggtgattcagcaagccctccggccccgccctccaggcaaccagtcacgctagtccaccccagcgtatcgccggcccttcccgcgctggctttagcggcaggctcgccgcctgatccggcgcgccctcgccgccgccaccgagaatcttcgcagcagccgcaccgccgggaagtgtactgggcgaggggcagcaccgtgtcgcagcaaatgtgcgaggcgcgcaggaaacgcaggcatgccgctggaatccacgcggacaatggaggggaaagggggggattcgttttggtggcccccagcgtagatggggcagggtgaccaggtctccttttttccttcaactgcccagattcgtccgaggttgggcattccttgcattacgggaaataccgcgcggctctgtaatgccggttgcgatttacgccttgtacttcaaattattgtttttcggggaaacccaccaggctgggttcgctagcgaagcgtccaagcctgactgagtagctgcctgcctggtcaccacttgaggtccaccacaccgcagtcacttgccctgccgccatggcggttctgagtgctgccatctctgcctctgcagctgccaggcaggctcgccatctgcctgctgtacgccagcacatctgacctccccccgcccccctgtcttcctctctcataggaacatcctctaggacgaaaagactttgagacagctctacctgtcgaggaaaa

SEQ ID NO: 95 - DNA, 329 bp, Tetraselmis sp.
  misc_feature 1..329: note = RB40 terminator
  source 1..329: mol_type = unassigned DNA
gcgctgtgcgcctagctttgcctttgcaccaacgatgacggcgatgctctgcatgtaccccgcgcgggtgtatgtaaatccatgtgccggagagcacttcgctccttcatcacactttttcgccatccgccattccccgccccagcctgaccaacctctatgccccaccacccgggactggcatgcctgaccctttcacatgcgcggtcggccttgggtcttctctctacacaagcggcgacttgctcgccaaagaggataggtcaaaggtcagtgaacatgttctctatatcacgctgtcggcgggtgttccactcggtctgtcgaaa

SEQ ID NO: 96 - DNA, 570 bp, synthetic construct
  misc_feature 1..570: note = Synthetic
  misc_feature 1..570: note = nourseothricin resistance gene codon optimized for Tetraselmis
  source 1..570: mol_type = other DNA
atgaccacactcgacgacaccgcatacagataccgcacaagcgttcctggcgacgcagaggcaattgaggcattagatggcagcttcaccaccgacaccgtgtttagagtgacagcaaccggcgacggctttacacttagagaggttccagtggacccgccattgacaaaggtgtttccagacgacgagagcgacgatgagagcgatgctggtgaagatggcgacccagatagccgcacatttgtggcatacggcgatgatggcgaccttgctgggtttgtggtggtgagctactctggctggaatagacgcttgaccgtggaggatatcgaggttgcaccagagcatagaggccatggagtgggtagagcactgatgggtctggcaaccgagtttgcaagggagagaggtgctggtcatctgtggttggaggtgacaaacgtgaacgcaccagcgatccacgcatacagaagaatgggcttcacgctgtgcggcctggatacagcactgtatgatggcacagcgagcgatggtgagcaagcactgtacatgagcatgccatgcccgtaa

SEQ ID NO: 97 - DNA, 20 bp, synthetic construct
  misc_feature 1..20: note = Synthetic
  misc_feature 1..20: note = Primer MCA1818
  source 1..20: mol_type = other DNA
ggcggcatgtctgcgcaagc

SEQ ID NO: 98 - DNA, 24 bp, synthetic construct
  misc_feature 1..24: note = Synthetic
  misc_feature 1..24: note = Primer MCA1819
  source 1..24: mol_type = other DNA
tttcgacagaccgagtggaacacc

(19) United States
(12) Patent Application Publication
(10) Pub. No.: US 2024/0052001 A1
(43) Pub. Date: Feb. 15, 2024

(54) HIGH PRODUCTIVITY ALGAL MUTANTS HAVING REDUCED PHOTOSYNTHETIC ANTENNA

(71) Applicant: Viridos, Inc., La Jolla, CA (US)

(72) Inventors: Eric R. Moellering, San Diego, CA (US); Nicholas Bauman, San Diego, CA (US); Randor R. Radakovits, Poway, CA (US); Roberto Spreafico, San Diego, CA (US); Fedor Kuzminov, San Diego, CA (US); Imad Ajjawi, San Diego, CA (US); Saheed Imam, San Diego, CA (US); Andrew Schultz, San Diego, CA (US); Kathleen Kwok, San Diego, CA (US); Moena Aqui, San Diego, CA (US); Jennifer Nominati, San Diego, CA (US); John Verruto, San Diego, CA (US); Shaun Bailey, San Diego, CA (US)

(21) Appl. No.: 18/475,792

(22) Filed: Sep. 27, 2023

Related U.S. Application Data
(62) Division of application No. 17/178,179, filed on Feb. 17, 2021, now Pat. No. 11,787,843.
Application No. 17/178,179 is a division of application No. 15/859,094, filed on Dec. 29, 2017, now Pat. No. 10,968,259.
(60) Provisional application No. 62/441,002, filed on Dec. 30, 2016.

(51) Int. Cl. C07K 14/405; C12N 1/36; C12N 1/12; C12N 15/82; C12P 21/00; C12N 15/01; A01H 1/06; C12P 7/6445; C12P 7/6454; C12P 7/649

(52) U.S. Cl. CPC C07K 14/405; C12N 1/36; C12N 1/12; C12N 15/8269; C12P 21/00; C12N 15/01; A01H 1/06; C12N 1/125; C12P 7/6445; C12P 7/6454; C12P 7/649; Y02E 50/10; C12R 2001/89

(57) ABSTRACT

Disclosed herein are mutant photosynthetic microorgnaisms having an attenuated SGI1 gene. The mutants have reduced chlorophyll and increased productivity with respect to wild type cells. Also disclosed are methods of using such mutants for producing biomass or bioproducts, and methods of screening for such mutants.

### CROSS REFERENCE TO RELATED APPLICATIONS

This application is a divisional application of U.S. application Ser. No. 17/178,179 filed Feb. 17, 2021, now pending; which is a divisional application of U.S. application Ser. No. 15/859,094 filed Dec. 29, 2017, now issued as U.S. Pat. No. 10,968,259; which claims the benefit under 35 USC § 119(e) to U.S. Application Ser. No. 62/441,002 filed Dec. 30, 2016, now expired. The disclosure of each of the prior applications is considered part of and is incorporated by reference in the disclosure of this application.

### INCORPORATION BY REFERENCE OF SEQUENCE LISTING

The material in the accompanying sequence listing is hereby incorporated by reference into this application. The accompanying sequence listing xml file, named SGI2080-3_ST26.xml, was created on Sep. 22, 2023 and is 198,757 bytes in size.

### BACKGROUND OF THE INVENTION

### Field of the Invention

The present invention relates to mutants of photosynthetic organisms having reduced chlorophyll and increased biomass and lipid productivity. The present invention also relates to methods of generating, selecting for, and screening for such mutants.

### Background Information

Engineering photosynthetic organisms to increase photosynthetic efficiency for higher productivity is a long-standing goal of plant and algal biologists. US 2014/0220638 and US 2016/030489, both of which are incorporated herein by reference, describe mutant screens to obtain algal mutants referred to as “LIHLA” mutants having reduced chlorophyll that are impaired in their ability to low light acclimate, that is, they retain the low chlorophyll state of high light adapted cells even in low light. US 2014/0220638 describes algal mutants having mutations in the Light Acclimation Regulator LAR1, LAR2, and LAR3 genes, and US 2016/0304896 discloses algal mutants having mutations in the chloroplastic SRP54 gene.

### SUMMARY OF THE INVENTION

Provided herein are mutant photosynthetic organisms that have an attenuated gene encoding a Significant Growth Improvement 1 (SGI1) polypeptide. The mutant photosynthetic organisms have higher productivity than control photosynthetic organisms that are substantially identical to the mutant photosynthetic microorganisms except that they do not have an attenuated SGI1 gene. Higher productivity can be measured as the rate of biomass accumulation or the rate of production of a bioproduct, such as, for example, lipid, protein, one or carbohydrates (e.g., one or more sugars or alcohols), pigments, antioxidants, terpenoids, vitamins, or polymers. The SGI1 mutants disclosed herein can exhibit increased productivity under photoautotrophic conditions.

As described herein, an SGI1 polypeptide is a polypeptide that has a Response Regulator receiver domain (RR domain) and a myb-like DNA-binding (myb) domain, where the RR domain is N-terminal to the myb domain, and the two domains are separated by a sequence of amino acids, referred to herein as a linker, that does not belong to either domain. The RR domain and myb domain in various embodiments can be in the N-terminal half of the protein.

An attenuated SGI1 gene is an SGI1 gene whose function or expression is disrupted such that the gene product (the SGI1 polypeptide) is reduced in amount or activity with respect to its abundance or activity in a wild type organism, that is, the function of the SGI1 polypeptide is diminished due to mutation of the SGI1 gene, or due to genetic manipulations outside the SGI1 gene itself that affect the expression of the SGI1 gene.

A mutant photosynthetic organism as provided herein that has an attenuated SGI1 gene exhibits higher productivity with respect to a control photosynthetic organism and has reduced chlorophyll with respect to the control photosynthetic organism. In some examples, a mutant photosynthetic organism having an attenuated SGI1 gene has a reduced amount of chlorophyll b, and in some examples, a mutant photosynthetic organism having an attenuated SGI1 gene has an increased chlorophyll a:chlorophyll b ratio. A mutant photosynthetic organism as provided herein that has an attenuated SGI1 gene can have decreased photosynthetic antenna size, for example reduced photosystem II (PSII) and/or reduced photosystem I (PSI) antenna size.

The high productivity SGI1 mutants can produce more biomass than a control photosynthetic organism, for example, more biomass per day than control or wild type photosynthetic organism from which the mutant photosynthetic organisms were derived. Biomass productivity can be ash free dry weight (AFDW), dry weight, wet weight, or total organic carbon (TOC) productivity. Alternatively or in addition, an SGI1 mutant can produce more of a bioproduct, such as but not limited to lipid, protein, carbohydrate, one or more polyketides, a terpenoid, a pigment, an antioxidant, a vitamin, one or more nucleotides, one or more nucleic acids, one or more amino acids, one or more carbohydrates, an alcohol, a hormone, a cytokine, a peptide, or a polymer than is produced by a control organism cultured under substantially the same conditions over the same period of time.

In some embodiments, a mutant photosynthetic organism as provided herein produces at least 10%, at least 15%, at least 20%, at least 25%, at least 30%, at least 35%, at least 40%, at least 45%, at least 50%, at least 60%, at least 70%, at least 80%, at least 90%, or at least 100% more biomass than a control photosynthetic organism cultured or cultivated under the substantially the same conditions, which for algae can be batch, semi-continuous, or continuous culture conditions and may be nutrient replete culture conditions or may be nitrogen deplete conditions, and may be photoautotrophic conditions.

The high productivity SGI1 mutants can have a reduced functional absorption cross section of PSII ((rpm′) or “reduced PSII antenna size”. For example, the cross-sectional unit size of the PSII antenna can be reduced by at least about 10%, at least 20%, at least 30%, at least 25%, at least 30%, at least 35%, at least 40%, at least 45%, at least 50%, at least 60%, at least about 70%, or at least about 80% with respect to the PSII antenna size of the control microorganism. An STI1 mutant can additionally have a reduced functional absorption cross section of PSI (σ_{PSI}). For example, the cross-sectional unit size of the PSI antenna can be reduced by at least 10%, at least 20%, at least 30%, at least 25%, at least 30%, at least 35%, at least 40%, at least 45%, at least 50%, or at least 60% with respect to the PSI antenna size of a control photosynthetic organism.

In various embodiments, a mutant photosynthetic organism as provided herein has increased Fv/Fm with respect to a control photosynthetic organism. For example, the mutant photosynthetic organism may have Fv/Fm increased by at least 5%, at least 10%, at least 12%, at least 15%, at least 20%, at least 30%, at least 40% or at least 50% with respect to a control photosynthetic organism, for example, increased by between about 5% and about 50%, or between about 5% and 30%, with respect to a control photosynthetic organism.

Further, a mutant photosynthetic organism as provided herein can have an increased rate of electron transport on the acceptor side of photosystem II (1/τ′_{Qa}) with respect to a control or wild type cell. A mutant as provided herein can have a 1/τ′_{Qa} value that is increased by at least about 20%, 30%, 40%, 50%, 60%, 80%, or 100% with respect to a wild type or control organism.

In addition, the rate of carbon fixation (Pmax (C)) in an SGI1 mutant as provided herein can be elevated with respect to a control organism. For example, Pmax (¹⁴C) can be increased by at least about 20%, 30%, 40%, 50%, 60%, 80%, or 100% with respect to a wild type or control organism.

In some embodiments, SGI1 mutants have decreased PSI and/or PSII antenna size and can further have a higher amount of a ribulose bisphosphate carboxylase activase (Rubisco activase or “RA”) than a control or wild type organism, for example, at least 1.2, 1.4, 1.6, 1.8, 2, 2.2, or 2.5 fold the amount of RA as a control organism. In some embodiments, the mutants demonstrate reduced expression of 6, 8, 10, 12, or 14 LHCP genes and increased expression of an RA gene, such as an RA-α or RA-β gene. Disclosed herein are mutant photosynthetic organisms having reduced chlorophyll and reduced reduced PSII antenna size where the mutants have a higher amount of Rubisco activase than control photosynthetic organisms. The mutants can have an attenuated SGI1 gene, and can have higher productivity than control photosynthetic organisms.

An SGI1 gene encodes a polypeptide having a response receiver (RR) domain and a myb domain, where the myb domain is C-terminal to the RR domain. In some embodiments the gene encodes a polypeptide that has the domain architecture (beginning from the N-terminal end) RR-linker-myb, where the linker is an amino acid sequence that is not part of the RR domain or the myb domain. The linker can optionally include a nuclear localization sequence (NLS). An SGI1 polypeptide can be a polypeptide having an RR domain N-terminal to a myb domain and separated from the myb domain by a linker that does not belong to either domain, where the polypeptide has a score of at least 350 when scanned with a Hidden Markov Model (HMM) that recognizes the SGI1 domain architecture of algal SGI1 polypeptides, as set forth in Example 6. Examples of such SGI polypeptides are provided in Tables 3 and 4. In various examples, the gene that is attenuated in a mutant as provided herein can (in a wild type organism) encode an SGI1 polypeptide that has a myb domain having at least 55%, at least 60%, at least 65%, at least 70%, at least 75%, at least 80%, at least 85%, at least 90%, or at least 95% amino acid sequence identity to a myb domain such as SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, or SEQ ID NO:75, and/or that includes an RR domain having at least 50%, at least 55%, at least 60%, at least 65%, at least 70%, at least 75%, at least 80%, at least 85%, at least 90%, or at least 95% amino acid sequence identity to an RR domain such as SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, or SEQ ID NO:57. Alternatively or in addition, the gene that is attenuated in a mutant as provided herein can (in a wild type organism) encode a polypeptide that includes an amino acid sequence having at least 50%, at least 55%, at least 60%, at least 65%, at least 70%, at least 75%, at least 80%, at least 85%, at least 90%, or at least 95% amino acid sequence identity to an SGI1 polypeptide such as SEQ ID NO:3, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, or SEQ ID NO:39.

Provided herein in some embodiments are mutant algal microorganisms having an attenuated SGI1 gene, where the mutants have higher biomass productivity, or produce more of a bioproduct, than a control algal microorganisms. The algal mutants can have reduced chlorophyll and can have reduced PSII antenna size. In some examples, the SGI1 gene that is attenuated can encode an SGI1 polypeptide that has a myb domain having at least 55%, at least 60%, at least 65%, at least 70%, at least 75%, at least 80%, at least 85%, at least 90%, or at least 95% amino acid sequence identity to a myb domain such as SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, and/or includes an RR domain having at least 50%, at least 55%, at least 60%, at least 65%, at least 70%, at least 75%, at least 80%, at least 85%, at least 90%, or at least 95% amino acid sequence identity to an RR domain such as SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49. Alternatively or in addition, the gene that is attenuated in a mutant alga as provided herein can (in a wild type alga) encode a polypeptide that includes an amino acid sequence having at least 50%, at least 55%, at least 60%, at least 65%, at least 70%, at least 75%, at least 80%, at least 85%, at least 90%, or at least 95% amino acid sequence identity to an SGI1 polypeptide such as SEQ ID NO:3, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, or SEQ ID NO:19.

Further provided herein are algal mutants having an attenuated SGI1 gene that have a higher protein content per unit biomass than the progenitor algae from which they are derived. The high protein content algae can have, for example, at least 5%, at least 10%, at least 15%, or at least 20% more protein on a per biomass basis than a control or wild type alga. In some embodiments the algal SGI1 mutants having increased protein content demonstrate a higher growth rate and/or a higher biomass productivity that the control or wild type algal strains from which they are derived. Also included is a high protein algal biomass, in which the algal biomass comprises mutant algal cells having an attenuated SGI1 gene, where the mutant algal cells have a higher protein content than the conrol or wild type alga from which they are derived. Further provided is a lysate of algal SGI1 mutant cells. The lysate can optionally have a higher protein content that an algal lysate of wild type algal cells.

A mutant algal microorganisms having an attenuated SGI1 gene can produce more biomass or more of a bioproduct than is produced by a control organism cultured under the substantially the same conditions. The bioroduct can be, for example, biomass, lipid, protein, or a carbohydrate. In various embodiments, an SGI1 mutant can produce more of one or more lipids, one or more proteins, one or more polyketides, a terpenoid, a pigment, an antioxidant, a vitamin, one or more nucleotides, one or more nucleic acids, one or more amino acids, one or more carbohydrates, an alcohol, a hormone, a cytokine, a peptide, or a polymer than is produced by a control alga that does not have an attenuated SGI1 gene. In some embodiments, the mutant alga having an attenuated SGI1 gene produces at least 10%, at least 15%, at least 20%, at least 25%, at least 30%, at least 35%, at least 40%, at least 45%, at least 50%, at least 60%, at least 70%, at least 80%, at least 90% or at least 100% more of a bioproduct than a control alga cultured under the substantially the same conditions, which can be batch, semi-continuous, or continuous culture conditions and may be nutrient replete culture conditions or may be nitrogen deplete conditions, and may be photoautotrophic conditions. Biomass can be measured as, for example, ash free dry weight (AFDW) or total organic carbon (TOC).

Further, an algal mutant having an attenuated SGI1 gene and having higher productivity with respect to a control or wild type alga can have reduced chlorophyll with respect to a control or wild type alga. For example, total chlorophyll may be reduced by about 20% to about 80% with respect to wild type levels, for example, by about 30% to about 70% of wild type levels.

A mutant algal microorganism having an attenuated SGI1 gene as provided herein can have a reduced photosystem II (PSII) antenna size. For example, the cross-sectional unit size of the PSII antenna can be reduced by at least 10%, at least 20%, at least 30%, at least 25%, at least 30%, at least 35%, at least 40%, at least 45%, at least 50%, or at least 60% with respect to the PSII antenna size of the control microorganism.

An algal mutant as provided herein having an attenuated SGI1 gene and demonstrating higher productivity than a control microorganism can optionally additionally have a reduced photosystem I (PSI) antenna. For example, the cross-sectional unit size of the PSII antenna can be reduced by at least 10%, at least 20%, at least 30%, at least 25%, at least 30%, at least 35%, at least 40%, at least 45%, at least 50%, or at least 60% with respect to the PSII antenna size of the control microorganism.

Additionally, a mutant alga as provided herein having an attenuated SGI1 gene can have increased carbon absorption, indicative of the rate of photosynthesis, with respect to a control alga, e.g., increased Pmax (C) with respect to a control alga. Pmax (C) can be increased by about by at least 10%, at least 20%, at least 25%, at least 30%, at least 40%, at least 45%, or at least 50% with resepect to the Pmax (C) of a control microorganism.

An algal mutant as provided herein having an attenuated SGI1 gene can further have reduced expression of LHC genes. For example, at least 6, at least 8, at least 10, or at least 12 LHC genes can be downregulated with respect to their expression level in a control cell. The reduction in expression of the LHC genes can be, for example, at least a 20%, 30%, 40%, 50%, 60%, or 70% reduction in the level of LHC transcripts.

Further, an algal mutant as provided herein having an attenuated SGI1 gene can have increased abundance of a Rubisco Activase (RA) polypeptide. The level of RA protein can be, for example, 1.5 fold, 2 fold, 2.5 fold, or greater than 2.5 fold the level of a control or wild type cell. One or both of an RA alpha isozyme or an RA beta isozyme can be increased in a high productivity mutant as provided herein. The level of an RA transcript can also be elevated in an algal mutant as provided herein, for example, from about 1.2 fold to 10 fold wild type levels or more. Provided herein in some embodiments is a low chlorophyll algal mutant having reduced PSII antenna size and increased expression of RA. The reduction in PSII antenna size can be for example, at least 20% or at least 30% with respect to wild type PSII antenna size, and the increase in RA protein level can be at least two-fold wild type.

In various embodiments a high productivity algal mutant as provided herein that has an attenuated SGI1 gene has at least 10%, at least 12%, at least 14%, at least 15%, at least 17%, or at least 20% more protein on a per TOC basis than a control alga. For example, the high productivity algal mutant can produce at least 10%, at least 20%, at least 25%, at least 30%, at least 35%, at least 40%, at least 45%, at least 50%, of at least 60% more protein than a control cell on a daily basis in a batch, continuous, or semi-continuous culture system.

A mutant algal microorganism as provided herein can be any eukaryotic microoalga, such as but not limited to a Chlorophyte or Charyophyte alga. For example, the mutant microalga can be a Chlorophyte alga of the the Chlorophyceace, the Chlorodendrophyceae, the Prasinophyceace, or the Trebouxiophyceae class. In some embodiments, the mutant microalga can be a member of the Chlorophyceace, such as a species of Asteromonas, Ankistrodesmus, Carteria, Chlamydomonas, Chlorococcum, Chlorogonium, Chrysosphaera, Dunaliella, Haematococcus, Monoraphidium, Neochloris, Oedogonium, Pelagomonas, Pleurococcus, Pyrobotrys, Scenedesmus, or Volvox. In alternative embodiments, the mutant microalga can be a member of the Chlorodendrophyceae, such as a species of Prasinocladus, Scherffelia, or Tetraselmis. In further alternative embodiments, the mutant alga can be a member of the Prasinophyceace, optionally a species of Ostreococcus or Micromonas. Further alternatively, the mutant microalga can be a member of the Chlorodendrophyceae, such as a species of Prasinocladus, Scherffelia, or Tetraselmis, or can be a member of the Trebouxiophyceae, optionally a species of a genus selected from the group consisting of Botryococcus, Chlorella, Auxenochlorella, Heveochlorella, Marinichlorella, Parachlorella, Pseudochlorella, Tetrachlorella, Eremosphaera, Franceia, Micractinium, Nannochloris, Oocystis, Picochlorum, Prototheca, Stichococcus, or Viridiella.

A futher aspect of the invention is a method of making a bioproduct, where the method includes culturing an mutant photosynthetic mutant as provided herein that has an attenuated SGI1 gene to produce the bioproduct. The method can further include recovering the bioproduct from the culture or organism. For example, the product can be recovered from the culture medium, harvested algal cells, or whole culture, or can be isolated from plants by harvesting or homogenizing plant tissue, for example. The bioproduct can be, for example, biomass, protein, lipid, or carbohydrate. Further, the product can be one or more lipids, proteins, polyketides, terpenoids, pigments, antioxidants, vitamins, nucleotides, nucleic acids, amino acids, carbohydrates, alcohols, hormones, cytokines, peptides, or polymers. In some embodiments, the product is a high protein biomass, where protein constitutes at least 50% of the TOC of the photosynthetic organism or tissue therefrom. In alternative embodiments, the product is lipid, and may be, for example, triglyceride.

The method can include culturing an algal SGI1 mutant in batch, semi-continuous, or continuous culture where the culture medium may be nutrient replete, or may be, for example, nitrogen deplete. The culturing can be under photoautotrophic conditions, in which inorganic carbon, e.g., carbon dioxide or carbonate, is substantially the sole carbon source in the culture medium for the production of organic molecules by the algal mutant.

Yet another aspect of the invention is a method of isolating a mutant alga having higher productivity with respect to the progenitor alga from which it is derived. The method includes: culturing an algal strain under semi-continuous or continuous photoautotrophic conditions for at least twenty generations and isolating at least one mutant line from the culture, where the isolated line has a higher growth rate than the progenitor algal strain. The method can include a mutagenesis step prior to the culturing. Mutagenesis can be by UV, gamma irradiation, chemical mutagens, random insertional mutagenesis, or targeted mutagenesis, for example. In various embodiments, the algae are exposed to constant light during the culturing. In various embodiments, the light intensity to which the algae are exposed during the culturing is at least 500 uE, at least 600 uE, at least 700 uE, at least 800 uD, at least 900 uE, at least 1000 uE, at least 1100 uE, at least 1200 uD, at least 1400 uE, at least 1600 uE, at least 1800 uE, at least 2000 uE, at least 2200 uE, at least 2500 uE, or at least 3000 uE.

The isolated line having a higher growth rate can be assayed for higher productivity with respect to the progenitor line, for example, in batch, semicontinuous, or continuous culture. Isolating a high productivity mutant can optionally further include: measuring one or more of Fv/Fm, sigma, PSII, PSI, or Pmax (C) (Pmax for carbon fixation).

The method can further include sequencing the genome of the isolated high productivity mutant to identify the mutation.

Yet another aspect of the invention is a method of selecting for high productivity algal mutants, where the method includes: mutagenizing a population of algae, selecting low chlorophyll cells using fluorescence activated cell sorting (FACS), distributing isolated lines of the selected low chlorophyll fluorescence cells in multiwell plates, screening the low chlorophyll fluorescence cells in multiwell plates for photosystem II (PSII) antenna size, Fv/Fm, and selecting at least one cell line having reduced PSII antenna size, increased Fv/Fm, and increased with respect to wild type cells, to select a high productivity mutant. The method can optionally further include measuring Pmax. The isolated line having a higher growth rate can be assayed for higher productivity with respect to the progenitor line, for example, in batch, semicontinuous, or continuous culture. The method can further include performing PCR and/or sequencing at least a portion of the genome of the isolated high productivity mutant to identify the mutation.

### BRIEF DESCRIPTION OF THE DRAWINGS

FIGS. 1A-1B provide graphs showing TOC productivity improvement of Parachlorella classical mutants FIG. 1A) NE-7843 and FIG. 1B) NE-13380 over WT-1185 under continuous light semicontinuous culture conditions (65% daily dilution, 2000 uE, 1% CO2). The represented data is from biological duplicate cultures.

FIG. 2 is a diagram of the Parachlorella T-2261 (SGI1) gene structure depicting locations of SNPs detected by genome sequencing of NE-7843 and NE-13380. The location targeted by a guide RNA for Cas9-mediated insertional mutagenesis is also shown.

FIG. 3 is a graph showing TOC productivities of Parachlorella T-2261 (SGI1) knockout mutants generated by Cas9. Data represent the average and standard deviation from three biological replicates assessed under continuous light (2000 μE) semicontinuous culture conditions.

FIG. 4 is an alignment of the Response Regulator receiver (RR) domain of algal SGI1 polypeptides. The sequences shown are Parachlorella sp. WT-1185 (SEQ ID NO:58), Coccomyxa subellipsoidea (SEQ ID NO:59), Ostreococcus lucimarinus (SEQ ID NO:60), Chlamydomonas reinhardtii (SEQ ID NO:61), Chromochloris zofingiensis (SEQ ID NO:62), Volvox carteri (SEQ ID NO:63), Tetraselmis sp. 105 (SEQ ID NO:64), Oocystis sp. (SEQ ID NO:65), and Micromonas sp. RCC299 (SEQ ID NO:66).

FIG. 5 is an alignment of the myb domain of algal SGI1 polypeptides. The sequences shown are Parachlorella sp. WT-1185 (SEQ ID NO:40), Coccomyxa subellipsoidea (SEQ ID NO:41), Ostreococcus lucimarinus (SEQ ID NO:42), Chlamydomonas reinhardtii (SEQ ID NO:43), Chromochloris zofingiensis (SEQ ID NO:44), Volvox carteri (SEQ ID NO:45), Tetraselmis sp. 105 (SEQ ID NO:46), Oocystis sp. (SEQ ID NO:47), and Micromonas sp. RCC299 (SEQ ID NO:48).

FIG. 6 is a schematic diagram of the FACS-FIRe screening method. Not shown is mutagenesis, which can be performed pior to FACS. The method can further include productivy testing.

FIG. 7 is a diagram of the Parachlorella SGI1 gene structure depicting locations of SNPs detected by genome sequencing of NE-07843 and NE-13380 and targeted mutationsGE-13371, GE13380, GE-16345, GE-16346, GE-16347, GE-16399, GE-13232, GE-13428, and GE-16980 resulting from Cas9-mediated mutagenesis. The locations of the guide RNAs are shown by arrows.

FIG. 8 is a graph showing the results of microproximate analysis of biomass of Parachlorella wild type strain WT-1185 and classical mutant NE-13380 to determine lipid, protein and carbohydrate content. Samples were acquired from cultures adapted to steady state 40% daily dilution in a semicontinuous system. FAMEs, carbohydrates, and amino acids are represented as the % of TOC allocated to each fraction calculated by the carbon content of each class of analyte. The inset numbers in the % C in protein indicate the actual percentage value.

FIG. 9 is a heat map based on transcriptomics data for 15 LHC genes of Parachlorella that (with the exception of CP26) are downregulated in the SGI1 mutant NE-7843 with respect to the parental wild type strain WT-1185.

FIGS. 10A-10D provide graphs showing the results of MRM analysis of PSII, PSI and Rubisco content of Parachlorella wild type (WT-1185) and SGI1 classical mutants (NE-7843 and NE-13380). FIG. 10A) the number of PSI, PSII, and Rubisco protein complexes per TOC in wild type WT-1185 and mutant NE-7843, FIG. 10B) the number of PSI, PSII, and Rubisco protein complexes per TOC in wild type WT-1185 and mutant NE-13380, and FIG. 10C) the number of the number of PSI, PSII, and Rubisco protein complexes in wild type WT-1185 and mutant NE-13380 on a per gram protein basis. FIG. 10D) Lower protein per TOC in MRM analysis compared to microproximate analysis (FIG. 8) to is related to non-ideal extraction efficiency.

FIG. 11A is a graph showing the difference in PSII, PSI and Rubisco Activase (RA) content of Parachlorella wild type (WT-1185) and SGI1 classical mutant NE-7843 by microanalysis.

FIG. 11B provides Western blots used for quantitating PSII, PSI and Rubisco Activase (RA) in Parachlorella wild type (WT-1185) and SGI1 classical mutant NE-7843.

FIG. 12 is a graph showing that SGI1 mutant NE-7843 exhibits increased biomass productivity under SCPA (semi-continuous) culturing in diel light modeled after a typical Imperial Valley, CA on May 4th day. Data represents biological triplicates.

FIG. 13 is a graph showing volumetric TOC accumulation under N-replete batch EPICs diel irradiance conditions. Biological duplicate data are shown individually for WT-1185 and NE-13380.

FIG. 14A is a graph showing volumetric TOC productivity under N-free batch AFS mode observed in strain NE-07843 as compared to the wild type stain WT-1185.

FIG. 14B is a graph showing volumetric FAME productivity under N-free batch AFS mode observed in strain NE-07843 as compared to the wild type stain WT-1185.

FIG. 15 provides diagrams of the Tetraselmis SGI1 alleles and the positions of guide target sequences MA1 (Guide 1) and JC2 (Guide 2).

FIGS. 16A-16C provide graphs showing chlorophyll content of Cas9-targeted SGI1 mutants on FIG. 16A) a per TOC basis and FIG. 16B) a per cell basis; FIG. 16C) provides chloropyll a:b ratios of the mutant clones.

FIGS. 17A-17D provide graphs showing photophysiological measurements of the Cas9 targeted Tetraslmis SGI1 mutants A) PSII cross-section measured at 450 nm, B) PSII cross-section measured at 520 nm, C) Fv/Fm, and D) PSII turnover rate.

FIGS. 18A-18C provide graphs showing photophysiological measurements of the Cas9 targeted Tetraslmis SGI1 mutants A) 14C Pmax per cell, B) 14C Pmax per TOC, and C) growth rate.

FIG. 19 is a graph providing the areal TOC productivity of two Cas9 targeted Tetraslmis SGI1 mutant strains, STR24096 amd STR24098 in a semi-continuous twenty-four hour light culture system.

FIG. 20A is a graph providing the areal TOC productivity of two Cas9 targeted Tetraslmis SGI1 mutant strains, STR24096 amd STR24098 in a semi-continuous diel culture system.

FIG. 20B is a graph providing the areal TOC productivity of a classically-mutagenized Parachlorella SGI1 mutant strain, NE-7843, in a semi-continuous diel culture system.

FIG. 21A is a photograph of wild type Arabidopsis plants (4 sectors on the left) and ARR2 mutant Arabidopsis plants (4 sectors on the right) grown for the same number of days

FIG. 21B is a graph showing the chlorophyll a and chlorophyll b content of the Arabidopsis wild type and ARR2 mutant plants as a percentage of wet weight.

FIG. 22A is a graph of the dry weight of Arabidopsis wild type and ARR2 mutant plants at 23 days

FIG. 22B is a graph of the wet weight of Arabidopsis wild type and ARR2 mutant plants at 23 days

FIG. 22C is a graph of the dry weight of Arabidopsis wild type and ARR2 mutant plants at 18 days.

FIG. 22D is a graph of the wet weight of Arabidopsis wild type and ARR2 mutant plants at 18 days.

### DETAILED DESCRIPTION OF THE INVENTION

Provided herein are algal mutants having reduced chlorophyll and higher photosynthetic efficiency with respect to control algae, in which the algal mutants have attenuated expression of a gene encoding a polypeptide having a Response Receiver Domain and a homeodomain. The bacterial CheY protein is a known member of a two component system that effects transcriptional change in response to environmental signals.

### Definitions

Unless defined otherwise, all technical and scientific terms used herein have the same meaning as commonly understood by one of ordinary skill in the art to which this invention belongs. In case of conflict, the present application including the definitions will control. Unless otherwise required by context, singular terms shall include pluralities and plural terms shall include the singular. All ranges provided within the application are inclusive of the values of the upper and lower ends of the range unless specifically indicated otherwise.

All publications, patents and other references mentioned herein are incorporated by reference in their entireties for all purposes as if each individual publication or patent application were specifically and individually indicated to be incorporated by reference.

The term “and/or” as used in a phrase such as “A and/or B” herein is intended to include “A and B”, “A or B”, “A”, and “B”.

“About” means either within 10% of the stated value, or within 5% of the stated value, or in some cases within 2.5% of the stated value, or, “about” can mean rounded to the nearest significant digit.

The term “gene” is used broadly to refer to any segment of a nucleic acid molecule (typically DNA, but optionally RNA) encoding a polypeptide or expressed RNA. Thus, genes include sequences encoding expressed RNA (which can include polypeptide coding sequences or, for example, functional RNAs, such as ribosomal RNAs, tRNAs, antisense RNAs, microRNAs, short hairpin RNAs, ribozymes, etc.). Genes may further comprise regulatory sequences required for or affecting their expression, as well as sequences associated with the protein or RNA-encoding sequence in its natural state, such as, for example, intron sequences, 5′ or 3′ untranslated sequences, etc. In some examples, “gene” may only refer to a protein-encoding portion of a DNA or RNA molecule, which may or may not include introns. A gene is preferably greater than 50 nucleotides in length, more preferably greater than 100 nucleotide in length, and can be, for example, between 50 nucleotides and 500,000 nucleotides in length, such as between 100 nucleotides and 100,000 nucleotides in length or between about 200 nucleotides and about 50,000 nucleotides in length, or about 200 nucleotides and about 20,000 nucleotides in length. Genes can be obtained from a variety of sources, including cloning from a source of interest or synthesizing from known or predicted sequence information.

The term “nucleic acid” or “nucleic acid molecule” refers to, a segment of DNA or RNA (e.g., mRNA), and also includes nucleic acids having modified backbones (e.g., peptide nucleic acids, locked nucleic acids) or modified or non-naturally-occurring nucleobases. The nucleic acid molecules can be double-stranded or single-stranded; a single stranded nucleic acid molecule that comprises a gene or a portion thereof can be a coding (sense) strand or a non-coding (antisense) strand.

A nucleic acid molecule or polypeptide may be “derived from” an indicated source, which includes the isolation (in whole or in part) of a nucleic acid segment or polypeptide from an indicated source. A nucleic acid molecule may also be derived from an indicated source by, for example, direct cloning, PCR amplification, or artificial synthesis from the indicated polynucleotide source or based on a sequence associated with the indicated polynucleotide source, which may be, for example, a species of organism.

Genes or nucleic acid molecules derived from a particular source or species also include genes or nucleic acid molecules having sequence modifications with respect to the source nucleic acid molecules, that is, the sequence of the gene or nucleic acid molecule is derived from the sequence of a gene or nucleic acid molecule from the referenced source or species but may have modifications. For example, a gene or nucleic acid molecule derived from a source (e.g., a particular referenced gene) can include one or more mutations with respect to the source gene or nucleic acid molecule that are unintended or that are deliberately introduced, and if one or more mutations, including substitutions, deletions, or insertions, are deliberately introduced the sequence alterations can be introduced by random or targeted mutation of cells or nucleic acids, by amplification or other gene synthesis or molecular biology techniques, or by chemical synthesis, or any combination thereof. A gene or nucleic acid molecule that is derived from a referenced gene or nucleic acid molecule that encodes a functional RNA or polypeptide can encode a functional RNA or polypeptide having at least 65%, at least 70%, at least 75%, at least 80%, at least 85%, at least 90%, or at least 95%, sequence identity with the referenced or source functional RNA or polypeptide, or to a functional fragment thereof. For example, a gene or nucleic acid molecule that is derived from a referenced gene or nucleic acid molecule that encodes a functional RNA or polypeptide can encode a functional RNA or polypeptide having at least 85%, at least 90%, at least 95%, at least 96%, at least 97%, at least 98%, or at least 99% sequence identity with the referenced or source functional RNA or polypeptide, or to a functional fragment thereof.

Similarly, a polypeptide or protein derived from a particular source or species includes polypeptides or proteins having sequence modifications with respect to the source prolypeptide, that is, the polpeptide is derived from the sequence of a polypeptide from the referenced source or species but may have modifications. For example, a polypeptide or protein derived from a source (e.g., a particular referenced protein) can include one or more mutations (amino acid differences) with respect to the source polypeptide that are unintended or that are deliberately introduced (for example, by mutation of the encoding nucleic acid molecule). A polypeptide that is derived from a referenced polypeptide can have at least 60%, at least 65%, at least 70%, at least 75%, at least 80%, at least 85%, at least 90%, or at least 95% sequence identity with the referenced or source polypeptide, or to a functional fragment thereof. For example, a polypeptide that is derived from a referenced polypeptide can have at least 80%, or at least 85%, at least 90%, at least 95%, at least 96%, at least 97%, at least 98%, or at least 99% sequence identity with the referenced or source polypeptide, or a functional fragment thereof.

As used herein, an “isolated” nucleic acid or protein is removed from its natural milieu or the context in which the nucleic acid or protein exists in nature. For example, an isolated protein or nucleic acid molecule is removed from the cell or organism with which it is associated in its native or natural environment. An isolated nucleic acid or protein can be, in some instances, partially or substantially purified, but no particular level of purification is required for isolation. Thus, for example, an isolated nucleic acid molecule can be a nucleic acid sequence that has been excised from the chromosome, genome, or episome that it is integrated into in nature.

A “purified” nucleic acid molecule or nucleotide sequence, or protein or polypeptide sequence, is substantially free of cellular material and cellular components. The purified nucleic acid molecule or protein may be substantially free of chemicals beyond buffer or solvent, for example. “Substantially free” is not intended to mean that other components beyond the novel nucleic acid molecules are undetectable.

The terms “naturally-occurring” and “wild type” refer to a form found in nature. For example, a naturally occurring or wild type nucleic acid molecule, nucleotide sequence or protein may be present in and isolated from a natural source, and is not intentionally modified by human manipulation.

As used herein “attenuated” means reduced in amount, degree, intensity, or strength. An attenuated gene is a gene that produces less of its encoded product and/or produces a less functional encoded product, with respect to a non-attenuated, or wild type, gene. An attneuated gene can in some examples produces none of its encoded product, or can produce a gene product (polypeptide) that is completely inactive. Attenuated gene expression may refer to a significantly reduced amount and/or rate of transcription of the gene in question, or of translation, folding, or assembly of the encoded protein. As nonlimiting examples, an attenuated gene may be a mutated or disrupted gene (e.g., a gene disrupted by partial or total deletion, truncation, frameshifting, or insertional mutation) that does not encode a complete functional open reading frame or that has decreased expression due to alteration or disruption of gene regulatory sequences. An attenuated gene can also be a gene that has one or more mutations affecting the amino acid sequence of the encoded polypeptide, where the encoded polypeptide is reduced in amount and/or activity by the mutation(s). An attenuated gene may also be a gene targeted by a construct that reduces expression of the gene, such as, for example, an antisense RNA, microRNA, RNAi molecule, or ribozyme. Attenuated gene expression can be gene expression that is eliminated, for example, reduced to an amount that is insignificant or undetectable. Attenuated gene expression can also be gene expression that results in an RNA or protein that is not fully functional or nonfunctional, for example, attenuated gene expression can be gene expression that results in a truncated RNA and/or polypeptide.

“Exogenous nucleic acid molecule” or “exogenous gene” refers to a nucleic acid molecule or gene that has been introduced (“transformed”) into a cell. A transformed cell may be referred to as a recombinant cell, into which additional exogenous gene(s) may be introduced. A descendent of a cell transformed with a nucleic acid molecule is also referred to as “transformed” if it has inherited the exogenous nucleic acid molecule. The exogenous gene or nucleic acid molecule may be derived from a different species (and so “heterologous”), or from the same species (and so “homologous”), relative to the cell being transformed. An “endogenous” nucleic acid molecule, gene or protein is a native nucleic acid molecule, gene, or protein as it occurs in, or is naturally produced by, the host.

The term “native” is used herein to refer to nucleic acid sequences or amino acid sequences as they naturally occur in the host. The term “non-native” is used herein to refer to nucleic acid sequences or amino acid sequences that do not occur naturally in the host. Thus, a “non-native” nucleic acid molecule is a nucleic molecule that is not naturally present in the host cell, for example, the non-native nucleic acid molecule is exogenous to the host cell or microorganism into which it is introduced, and may be heterologous with respect to the host cell or microorganism. Additionally, a nucleic acid sequence or amino acid sequence that has been removed from a cell, subjected to laboratory manipulation, and introduced or reintroduced into a host cell such that it differs in sequence or location in the genome with respect to its position in a non-manipulated organism (i.e., is juxtaposed with or operably linked to sequences it is not juxtaposed with or operably linked to in a non-transformed organism) is considered “non-native”. Non-native genes also include genes endogenous to the host microorganism operably linked to one or more heterologous regulatory sequences that have been recombined into the host genome.

A “recombinant” or “engineered” nucleic acid molecule is a nucleic acid molecule that has been altered through human manipulation. As non-limiting examples, a recombinant nucleic acid molecule includes any nucleic acid molecule that: 1) has been partially or fully synthesized or modified in vitro, for example, using chemical or enzymatic techniques (e.g., by use of chemical nucleic acid synthesis, or by use of enzymes for the replication, polymerization, digestion (exonucleolytic or endonucleolytic), ligation, reverse transcription, transcription, base modification (including, e.g., methylation), integration or recombination (including homologous and site-specific recombination) of nucleic acid molecules); 2) includes conjoined nucleotide sequences that are not conjoined in nature; 3) has been engineered using molecular cloning techniques such that it lacks one or more nucleotides with respect to the naturally occurring nucleic acid molecule sequence; and/or 4) has been manipulated using molecular cloning techniques such that it has one or more sequence changes or rearrangements with respect to the naturally occurring nucleic acid sequence. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector.

The term “recombinant protein” as used herein refers to a protein produced by genetic engineering regardless of whether the amino acid varies from that of a wild-type protein.

When applied to organisms, the term recombinant, engineered, or genetically engineered refers to organisms that have been manipulated by introduction of a heterologous or exogenous recombinant nucleic acid sequence into the organism (e.g., a non-native nucleic acid sequence), and includes gene knockouts, targeted mutations, gene replacement, and promoter replacement, deletion, disruption, or insertion, as well as introduction of transgenes or synthetic genes or nucleic acid sequences into the organism. That is, recombinant, engineered, or genetically engineered refers to organisms that have been altered by human intervention. Recombinant or genetically engineered organisms can also be organisms into which constructs for gene “knockdown” have been introduced. Such constructs include, but are not limited to, RNAi, microRNA, shRNA, siRNA, antisense, and ribozyme constructs. Also included are organisms whose genomes have been altered by the activity of meganucleases, zinc finger nucleases, TALENs, or cas/CRISPR systems. An exogenous or recombinant nucleic acid molecule can be integrated into the recombinant/genetically engineered organism's genome or in other instances may not be integrated into the host genome. As used herein, “recombinant microorganism” or “recombinant host cell” includes progeny or derivatives of the recombinant microorganisms of the invention. Because certain modifications may occur in succeeding generations due to either mutation or environmental influences, such progeny or derivatives may not, in fact, be identical to the parent cell, but are still included within the scope of the term as used herein.

The term “promoter” refers to a nucleic acid sequence capable of binding RNA polymerase in a cell and initiating transcription of a downstream (3′ direction) coding sequence. A promoter includes the minimum number of bases or elements necessary to initiate transcription at levels detectable above background. A promoter can include a transcription initiation site as well as protein binding domains (consensus sequences) responsible for the binding of RNA polymerase. Eukaryotic promoters often, but not always, contain “TATA” boxes and “CAT” boxes. Prokaryotic promoters may contain−10 and−35 prokaryotic promoter consensus sequences. A large number of promoters, including constitutive, inducible and repressible promoters, from a variety of different sources are well known in the art. Representative sources include for example, algal, viral, mammalian, insect, plant, yeast, and bacterial cell types, and suitable promoters from these sources are readily available, or can be made synthetically, based on sequences publicly available on line or, for example, from depositories such as the ATCC as well as other commercial or individual sources. Promoters can be unidirectional (initiate transcription in one direction) or bi-directional (initiate transcription in either direction). A promoter may be a constitutive promoter, a repressible promoter, or an inducible promoter. A promoter region can include, in addition to the gene-proximal promoter where RNA polymerase binds to initiate transcription, additional sequences upstream of the gene that can be within 1 kb, 2 kb, 3 kb, 4 kb, 5 kb or more of the transcriptional start site of a gene, where the additional sequences can influence the rate of transcription of the downstream gene and optionally the responsiveness of the promoter to developmental, environmental, or biochemical (e.g., metabolic) conditions.

The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism species. In contrast a “homologous” polynucleotide, gene, nucleic acid, polypeptide, or enzyme is used herein to denote a polynucleotide, gene, nucleic acid, polypeptide, or enzyme that is derived from the host organism species. When referring to a gene regulatory sequence or to an auxiliary nucleic acid sequence used for maintaining or manipulating a gene sequence (e.g., a promoter, a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e., in the genome of a non-genetically engineered organism) is referred to herein as a “heterologous promoter,” even though the promoter may be derived from the same species (or, in some cases, the same organism) as the gene to which it is linked.

As used herein, the term “protein” or “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms.

Gene and protein Accession numbers, commonly provided in parenthesis after a gene or species name, are unique identifiers for a sequence record publicly available at the National Center for Biotechnology Information (NCBI) website (ncbi.nlm.nih.gov) maintained by the United States National Institutes of Health. The “GenInfo Identifier” (GI) sequence identification number is specific to a nucleotide or amino acid sequence. If a sequence changes in any way, a new GI number is assigned. A Sequence Revision History tool is available to track the various GI numbers, version numbers, and update dates for sequences that appear in a specific GenBank record. Searching and obtaining nucleic acid or gene sequences or protein sequences based on Accession numbers and GI numbers is well known in the arts of, e.g., cell biology, biochemistry, molecular biology, and molecular genetics.

As used herein, the terms “percent identity” or “homology” with respect to nucleic acid or polypeptide sequences are defined as the percentage of nucleotide or amino acid residues in the candidate sequence that are identical with the known polypeptides, after aligning the sequences for maximum percent identity and introducing gaps, if necessary, to achieve the maximum percent homology. N-terminal or C-terminal insertion or deletions shall not be construed as affecting homology, and internal deletions and/or insertions into the polypeptide sequence of less than about 30, less than about 20, or less than about 10 amino acid residues shall not be construed as affecting homology. Homology or identity at the nucleotide or amino acid sequence level can be determined by BLAST (Basic Local Alignment Search Tool) analysis using the algorithm employed by the programs blastp, blastn, blastx, tblastn, and tblastx (Altschul (1997), Nucleic Acids Res. 25, 3389-3402, and Karlin (1990), Proc. Natl. Acad. Sci. USA 87, 2264-2268), which are tailored for sequence similarity searching. The approach used by the BLAST program is to first consider similar segments, with and without gaps, between a query sequence and a database sequence, then to evaluate the statistical significance of all matches that are identified, and finally to summarize only those matches which satisfy a preselected threshold of significance. For a discussion of basic issues in similarity searching of sequence databases, see Altschul (1994), Nature Genetics 6, 119-129. The search parameters for histogram, descriptions, alignments, expect (i.e., the statistical significance threshold for reporting matches against database sequences), cutoff, matrix, and filter (low complexity) can be at the default settings. The default scoring matrix used by blastp, blastx, tblastn, and tblastx is the BLOSUM62 matrix (Henikoff (1992), Proc. Natl. Acad. Sci. USA 89, 10915-10919), recommended for query sequences over 85 in length (nucleotide bases or amino acids).

For blastn, designed for comparing nucleotide sequences, the scoring matrix is set by the ratios of M (i. e., the reward score for a pair of matching residues) to N (i.e., the penalty score for mismatching residues), wherein the default values for M and N can be +5 and −4, respectively. Four blastn parameters can be adjusted as follows: Q=10 (gap creation penalty); R=10 (gap extension penalty); wink=1 (generates word hits at every winkth position along the query); and gapw=16 (sets the window width within which gapped alignments are generated). The equivalent Blastp parameter settings for comparison of amino acid sequences can be: Q=9; R=2; wink=1; and gapw=32. A Bestfit comparison between sequences, available in the GCG package version 10.0, can use DNA parameters GAP=50 (gap creation penalty) and LEN=3 (gap extension penalty), and the equivalent settings in protein comparisons can be GAP=8 and LEN=2.

As used herein, the phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz (1979) Principles of Protein Structure, Springer-Verlag). According to such analyses, groups of amino acids can be defined where amino acids within a group exchange preferentially with each other, and therefore resemble each other most in their impact on the overall protein structure (Schulz (1979) Principles of Protein Structure, Springer-Verlag). Examples of amino acid groups defined in this manner can include: a “charged/polar group” including Glu, Asp, Asn, Gln, Lys, Arg, and His; an “aromatic or cyclic group” including Pro, Phe, Tyr, and Trp; and an “aliphatic group” including Gly, Ala, Val, Leu, Ile, Met, Ser, Thr, and Cys. Within each group, subgroups can also be identified. For example, the group of charged/polar amino acids can be sub-divided into sub-groups including: the “positively-charged sub-group” comprising Lys, Arg and His; the “negatively-charged sub-group” comprising Glu and Asp; and the “polar sub-group” comprising Asn and Gln. In another example, the aromatic or cyclic group can be sub-divided into sub-groups including: the “nitrogen ring sub-group” comprising Pro, His, and Trp; and the “phenyl sub-group” comprising Phe and Tyr. In another further example, the aliphatic group can be sub-divided into sub-groups including: the “large aliphatic non-polar sub-group” comprising Val, Leu, and Ile; the “aliphatic slightly-polar sub-group” comprising Met, Ser, Thr, and Cys; and the “small-residue sub-group” comprising Gly and Ala. Examples of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH2 can be maintained. A “conservative variant” is a polypeptide that includes one or more amino acids that have been substituted to replace one or more amino acids of the reference polypeptide (for example, a polypeptide whose sequence is disclosed in a publication or sequence database, or whose sequence has been determined by nucleic acid sequencing) with an amino acid having common properties, e.g., belonging to the same amino acid group or sub-group as delineated above.

As used herein, “expression” includes the expression of a gene at least at the level of RNA production, and an “expression product” includes the resultant product, e.g., a polypeptide or functional RNA (e.g., a ribosomal RNA, a tRNA, an antisense RNA, a micro RNA, an shRNA, a ribozyme, etc.), of an expressed gene. The term “increased expression” includes an alteration in gene expression to facilitate increased mRNA production and/or increased polypeptide expression. “Increased production” [of a gene product] includes an increase in the amount of polypeptide expression, in the level of the enzymatic activity of a polypeptide, or a combination of both, as compared to the native production or enzymatic activity of the polypeptide.

Some aspects of the present invention include the partial, substantial, or complete deletion, silencing, inactivation, or down-regulation of expression of particular polynucleotide sequences. The genes may be partially, substantially, or completely deleted, silenced, inactivated, or their expression may be down-regulated in order to affect the activity performed by the polypeptide they encode, such as the activity of an enzyme. Genes can be partially, substantially, or completely deleted, silenced, inactivated, or down-regulated by insertion of nucleic acid sequences that disrupt the function and/or expression of the gene (e.g., viral insertion, transposon mutagenesis, meganuclease engineering, homologous recombination, or other methods known in the art). The terms “eliminate,” “elimination,” and “knockout” can be used interchangeably with the terms “deletion,” “partial deletion,” “substantial deletion,” or “complete deletion.” In certain embodiments, a microorganism of interest may be engineered by site directed homologous recombination or targeted integration or mutation using a cas/CRISPR system to knockout a particular gene of interest. In still other embodiments, targeted insertion into or mutation of a gene regulatory region using a cas/CRISPR system, RNAi, or antisense DNA (asDNA) constructs may be used to partially, substantially, or completely silence, inactivate, or down-regulate a particular gene of interest.

These insertions, deletions, or other modifications of certain nucleic acid molecules or particular polynucleotide sequences may be understood to encompass “genetic modification(s)” or “transformation(s)” such that the resulting strains of the microorganisms or host cells may be understood to be “genetically modified”, “genetically engineered” or “transformed.”

As used herein, “up-regulated” or “up-regulation” includes an increase in expression of a gene or nucleic acid molecule of interest or the activity of an enzyme, e.g., an increase in gene expression or enzymatic activity as compared to the expression or activity in an otherwise identical gene or enzyme that has not been up-regulated.

As used herein, “down-regulated” or “down-regulation” includes a decrease in expression of a gene or nucleic acid molecule of interest or the activity of an enzyme, e.g., a decrease in gene expression or enzymatic activity as compared to the expression or activity in an otherwise identical gene or enzyme that has not been down-regulated.

As used herein, “mutant” refers to an organism that has a mutation in a gene that is the result of classical mutagenesis, for example, using gamma irradiation, UV, or chemical mutagens, or is a recombinant organism that has altered structure or expression of a gene as a result of genetic engineering that many include, as non-limiting examples, overexpression, including expression of a gene under different temporal, biological, or environmental regulation and/or to a different degree than occurs naturally and/or expression of a gene that is not naturally expressed in the recombinant cell; homologous recombination, including knock-outs and knock-ins (for example, gene replacement with genes encoding polypeptides having greater or lesser activity than the wild type polypeptide, and/or dominant negative polypeptides); gene attenuation via RNAi, antisense RNA, or ribozymes, or the like; and genome engineering using meganucleases, TALENs, and/or CRISPR technologies, and the like. A mutant is therefore not a naturally-occurring organism and is the result of human intervention. A mutant organism of interest will typically have a phenotype different than that of the corresponding wild type or progenitor strain that lacks the mutation, where the phenotype can be assessed by growth assays, product analysis, photosynthetic properties, biochemical assays, etc. When referring to a gene “mutant” means the gene has at least one base (nucleotide) change, deletion, or insertion with respect to a native or wild type gene. The mutation (change, deletion, and/or insertion of one or more nucleotides) can be in the coding region of the gene or can be in an intron, 3′ UTR, 5′ UTR, or promoter region, e.g., within 2 kb of the transcriptional start site or within 3 kb or the translational start site. For example, a mutant having attenuated expression of a gene as disclosed herein can have a mutation, which can be one or more nucleobase changes and/or one or more nucleobase deletions and/or one or more nucleobase insertions, into the region of a gene 5′ of the transcriptional start site, such as, in non-limiting examples, within 2 kb, within 1.5 kb, within 1 kb, or within 0.5 kb of the known or putative transcriptional start site, or within 3 kb, within 2.5 kb, within 2 kb, within 1.5 kb, within 1 kb, or within 0.5 kb of the translational start site. As nonlimiting examples, a mutant gene can be a gene that has a mutation, insertion, or deletion within the promoter region that can either increase or decrease expression of the gene; can be a gene that has a deletion that results in production of a nonfunctional protein, truncated protein, dominant negative protein, or no protein; can be a gene that has one or more point mutations leading to a change in the amino acid of the encoded protein or results in aberrant splicing of the gene transcript, etc.

Conserved domains of polypeptides include those identified in the “cd” (conserved domain) database, the COG database, the SMART database, the PRK database, the TIGRFAM database, the InterPro database (ebi.ac.uk/interpro/) or others known the art. The National Center for Biotechnology Information website (ncbi.nlm.nih.gov/Structure/cdd/wrpsb.cgi) sponsored by the U.S. National Institutes of Health includes a conserved domain database (CDD) which it describes as “a protein annotation resource that consists of a collection of well-annotated multiple sequence alignment models for ancient domains and full-length proteins. These are available as position-specific score matrices (PSSMs) for fast identification of conserved domains in protein sequences via RPS-BLAST. CDD content includes NCBI-curated domains, which use 3D-structure information to explicitly define domain boundaries and provide insights into sequence/structure/function relationships, as well as domain models imported from a number of external source databases (Pfam, SMART, COG, PRK, TIGRFAM).”

The term “Pfam” refers to a large collection of protein domains and protein families maintained by the Pfam Consortium and available at several sponsored world wide web sites, including: pfam.sanger.ac.uk/(Welcome Trust, Sanger Institute); pfam.sbc.su.se (Stockholm Bioinformatics Center); pfam.janelia.org/(Janelia Farm, Howard Hughes Medical Institute); pfam.jouy.inra.fr/(Institut national de la Recherche Agronomique); and pfam.ccbb.re.kr. The latest release of Pfam is Pfam 30.0 (May 2016). Pfam domains and families are identified using multiple sequence alignments and hidden Markov models (HMMs). Pfam-A family or domain assignments, are high quality assignments generated by a curated seed alignment using representative members of a protein family and profile hidden Markov models based on the seed alignment. (Unless otherwise specified, matches of a queried protein to a Pfam domain or family are Pfam-A matches.) All identified sequences belonging to the family are then used to automatically generate a full alignment for the family (Sonnhammer (1998) Nucleic Acids Research 26, 320-322; Bateman (2000) Nucleic Acids Research 26, 263-266; Bateman (2004) Nucleic Acids Research 32, Database Issue, D138-D141; Finn (2006) Nucleic Acids Research Database Issue 34, D247-251; Finn (2010) Nucleic Acids Research Database Issue 38, D211-222). By accessing the Pfam database, for example, using any of the above-reference websites, protein sequences can be queried against the HMMs using HMMER homology search software (e.g., HMMER2, HMMER3, or a higher version, hmmer.janelia.org/). Significant matches that identify a queried protein as being in a pfam family (or as having a particular Pfam domain) are those in which the bit score is greater than or equal to the gathering threshold for the Pfam domain. Expectation values (e values) can also be used as a criterion for inclusion of a queried protein in a Pfam or for determining whether a queried protein has a particular Pfam domain, where low e values (much less than 1.0, for example less than 0.1, or less than or equal to 0.01) represent low probabilities that a match is due to chance.

A “cDNA” is a DNA molecule that comprises at least a portion the nucleotide sequence of an mRNA molecule, with the exception that the DNA molecule substitutes the nucleobase thymine, or T, in place of uridine, or U, occurring in the mRNA sequence. A cDNA can be double stranded or single stranded and can be, for example, the complement of the mRNA sequence. In preferred examples, a cDNA does not include one or more intron sequences that occur in the naturally-occurring gene that the cDNA corresponds to (i.e., the gene as it occurs in the genome of an organism). For example, a cDNA can have sequences from upstream of an intron of a naturally-occurring gene juxtaposed to sequences downstream of the intron of the naturally-occurring gene, where the upstream and downstream sequences are not juxtaposed in a DNA molecule in nature (i.e., the sequences are not juxtaposed in the naturally occurring gene). A cDNA can be produced by reverse transcription of mRNA molecules, or can be synthesized, for example, by chemical synthesis and/or by using one or more restriction enzymes, one or more ligases, one or more polymerases (including, but not limited to, high temperature tolerant polymerases that can be used in polymerase chain reactions (PCRs)), one or more recombinases, etc., based on knowledge of the cDNA sequence, where the knowledge of the cDNA sequence can optionally be based on the identification of coding regions from genome sequences or compiled from the sequences multiple partial cDNAs.

Reference to properties that are “substantially the same” or “substantially identical” without further explanation of the intended meaning, is intended to mean the properties are within 10%, and preferably within 5%, and may be within 2.5%, of the reference value. Where the intended meaning of “substantially” in a particular context is not set forth, the term is used to include minor and irrelevant deviations that are not material to the characteristics considered important in the context of the invention.

A “control cell” “control organism” or “control microorganism” is either a wild type cell, organism, or microorganism from which a mutant cell, organism, or microorganism (genetically engineered or mutagenized cell, organism, or microorganism) is directly or indirectly derived, or is a cell, organism, or microorganism that is substantially identical to the mutant cell, organism, or microorganism referred to, with the exception that the control cell, organism, or microorganism does not have the mutation resulting in increased productivity, for example, the control cell, organism, or microorganism has not been genetically engineered or mutagenized to attenuate an SGI1 gene. For example, where the recombinant alga comprises mutation in an SGI1 gene, a control alga can be substantially identical to the recombinant alga with the exception that the control alga does not comprise a mutation in the SGI1 gene.

“The same conditions” or “the same culture conditions”, as used herein, means substantially the same conditions, that is, any differences between the referenced conditions that may be present are minor and not relevant to the function or properties of the microorganism that are material to the invention, including lipid production or biomass production.

As used herein “lipid” or “lipids” refers to fats, waxes, fatty acids, fatty acid derivatives such as fatty alcohols, wax esters, alkanes, and alkenes, sterols, monoglycerides, diglycerides, triglycerides, phospholipids, sphingolipids, saccharolipids, and glycerolipids. “FAME lipids” or “FAME” refers to lipids having acyl moieties that can be derivatized to fatty acid methyl esters, such as, for example, monoacylglycerides, diacylglycerides, triacylglycerides, wax esters, and membrane lipids such as phospholipids, galactolipids, etc. Lipid productivity can be assessed as FAME productivity in milligrams per liter (mg/L) and for algae, may be reported as grams per meter per day (g/m²/day). In the semi-continuous assays provided herein, mg/L values are converted to g/m2/day by taking into account the area of incident irradiance (the SCPA flask rack aperture of 1½″×3⅜″, or 0.003145 m²) and the volume of the culture (550 ml). To obtain productivity values in g/m²/day, mg/L values are multiplied by the daily dilution rate (30%) and a conversion factor of 0.175. Where lipid or subcategories thereof (for example, TAG or FAME) are referred to as a percentage, the percentage is a weight percent unless indicated otherwise.

As used herein, the term “fatty acid product” includes free fatty acids, mono-di- or triglycerdes, fatty aldehydes, fatty alcohols, fatty acid esters (including, but not limited to, wax esters); and hydrocarbons, including, but not limited to, alkanes and alkenes).

“Biomass” refers to cellular mass, whether of living or dead cells, and can be assessed, for example, as wet weight or aspirated pellet weight, or as dry weight (e.g., lyophilate of a culture sample or pelleted cells), ash-free dry weight (AFDW), or total organic carbon (TOC), using methods known in the art. Biomass increases during the growth of a culture under growth permissive conditions and may be referred to as “biomass accumulation” in batch cultures, for example. In continuous or semi-continuous cultures that undergo steady or regular dilution, biomass that is produced that would otherwise accumulate in the culture is removed during culture dilution. Thus, daily biomass productivity (increases in biomass) by these cultures can also be referred to as “biomass accumulation”. Biomass productivity can be assessed as TOC productivity in milligrams per liter (mg/L) and for algae, may be reported as grams per meter² per day (g/m²/day). In the semi-continuous assays provided herein, mg/L values are converted to g/m2/day by taking into account the area of incident irradiance (the SCPA flask rack aperture of 1½ ″×3⅜″, or 0.003145 m²) and the volume of the culture (550 ml). To obtain productivity values in g/m²/day, mg/L values are multiplied by the daily dilution rate (30%) and a conversion factor of 0.175. Where biomass is expressed as a percentage, the percentage is a weight percent unless indicated otherwise.

In the context of the invention, a “nitrogen source” is a source of nitrogen that can be taken up and metabolized by the subject microorganism and incorporated into biomolecules for growth and propagation. For example, compounds including nitrogen that cannot be taken up and/or metabolized by the microorganism for growth (e.g., nitrogen-containing biological buffers such as Hepes, Tris, etc.) are not considered nitrogen sources in the context of the invention.

“Reduced nitrogen”, as used herein, is nitrogen in the chemical form of ammonium, ammonia, urea, or an amino acid that can be taken up and metabolized by the microorganism being cultured to provide a source of nitrogen for incorporation into biomolecules, thereby supporting growth. For example, in addition to ammonium/ammonia and urea, reduced nitrogen can include various amino acids where the amino acid(s) can serve as a nitrogen source to the subject microorganism. Examples of amino acids can include, without limitation, glutamate, glutamine, histidine, lysine, arginine, asparagine, alanine, and glycine. “Non-reduced nitrogen” in the context of a nitrogen source that can be present in a culture medium for microorganisms refers to nitrate or nitrite that must be reduced prior to assimilation into organic compounds by the microorganism.

“The sole source of nitrogen [in the culture medium]” is used interchangeably with “substantially the sole source of nitrogen” and indicates that no other nitrogen source is intentionally added to the culture medium, or that no other nitrogen source is present in an amount sufficient to significantly increase the growth of the microorganisms or cells cultured in the referenced medium. Throughout this application, for brevity, the terms “nitrate-only” is used to characterize culture media in which nitrate is the only source of nitrogen that is available to the microorganisms for supporting growth.

Similarly, “the sole source of carbon [in the culture medium]” is used interchangeably with “substantially the sole source of carbon” and indicates that no other carbon source is present in an amount sufficient to increase the productivity, growth, or propagation of the microorganisms or cells cultured in the referenced medium or become incorporated into biomolecules such as lipids produced by the microorganisms or cells.

“Nitrogen replete” conditions refer to media conditions in which no further growth or propagation benefit is conferred by adding additional nitrogen (in a form that can be used by the microorganism) to the medium. Similarly, “nutrient replete” conditions refer to media conditions in which no nutrient is limiting to growth or propagation, that is, when a medium is nutrient replete, adding additional nutrient(s) to the medium does not result in an improved growth or propagation rate. In the context of “nutrient replete”, “nutrients” includes, as nonlimiting examples, phosphate, sulfur, iron, and optionally silica, but excludes carbon sources such as sugars or organic acids that may be used by the organism as an energy source.

### SGI1 Polypeptides

As described herein, SGI1 or “Significant Growth Improvement 1” polypeptides are polypeptides that include a Response Regulator receiver or “RR” domain (pfam PF00072) and a Myb-like binding domain, refferred to herein simply as a “myb” domain (pfam PF00249), where the RR domain is positioned N-terminal to the myb domain. The amino acid sequence of an SGI1 polypeptide that encompasses the RR domain and myb domain includes a stretch of amino acids that occurs between the RR and myb domains that is found to be poorly conserved or not conserved among SGI1 polypeptides. The amino acid sequence occurring between the RR domain and myb domain may be referred to herein as a linker between the two domains. The linker may be of any length, and in various examples may range in length from one to about 300 amino acids, from ten to about 200 amino acids, or from twenty to about 150 amino acids in length. The linker region can optionally include a nuclear localization sequence (NLS).

An RR domain within an SGI1 protein can be characterized as pfam PF00072, or as a “signal receiver domain” or simply “receiver domain”, and/or can be classified as cd00156 in the conseved domain database (CDD), as COG0784 in the Clusters of Orthologous Groups of proteins database, or as an Interpro “CheY-like superfamily” domain, IPR011006. The RR domain is found in bacterial two-component regulatory systems (like the bacterial chemotaxis two-component system that includes a polypeptide known as CheY), in which it receives a signal from a sensor partner. The RR domain of such systems is often found N-terminal to a DNA binding domain and can include a phosphoacceptor site. FIG. 4 provides an alignment of the RR domains of Parachlorella sp. WT-1185 (SEQ ID NO:58), Coccomyxa subellipsoidea (SEQ ID NO:59), Ostreococcus lucimarinus (SEQ ID NO:60), Chlamydomonas reinhardtii (SEQ ID NO:61), Chromochloris zofingiensis (SEQ ID NO:62), Volvox carteri (SEQ ID NO:63), Tetraselmis sp. 105 (SEQ ID NO:64), Oocystis sp. WT-4183 (SEQ ID NO:65), and Micromonas sp. RCC299 (SEQ ID NO:66).

A myb domain within an SGI1 protein can be characterized, for example, as pfamPF00249: “Myb-like DNA-binding domain”, and/or may be identified as conserved domain TIGRO1557 “myb-like DNA-binding domain, SHAQKYF class”, or as an Interpro Homeobox-like domain superfamily domain (IPR009057) and/or an Interpro Myb domain (IPR017930).

In addition to having an RR domain N-terminal to a myb domain, an SGI1 protein as provided herein can have a score of 300 or higher, 320 or higher, 340 or higher, 350 or higher, 360 or higher, or 370 or higher with an e-value of less than about 1e-10, 1e50, 1e-70, or 1e-100, when scanned with a Hidden Markov Model (HMM) designed to score proteins on the basis of how well a protein's amino acid sequence matches the conserved amino acids of a region of SGI1 homologs in algae (see Example 6). The region of SGI1 polypeptides used to develop the HMM is the amino acids sequence that includes (proceeding in the N-terminal to C-terminal direction) the RR domain, the linker, and the myb domain. In an HMM, highly conserved amino acid positions are weighted more heavily than poorly conserved amino acid positions within a compared region of the polypeptides to arrive at the score. Polypeptides having scores of at least about 300, or of 350 or greater, such as for example 370 or greater, when scanned with an HMM model based on protein sequences of algal SGI1 polypeptides that include a single continuous sequence that includes the RR domain, linker, and myb domain developed using include, without limitation, polypeptides of the algal and plant species Parachlorella sp. 1185 (SEQ ID NO:3), Coccomyxa subellipsoidea (SEQ ID NO:9), Ostreococcus lucimarinus (SEQ ID NO:10), Chlamydomonas reinhardtii (SEQ ID NO:11), Volvox carteri (SEQ ID NO:13), Tetraselmis sp. 105 (SEQ ID NOs:14, 15, and 16), Oocystis sp. (SEQ ID NO:17), Micromonas sp. RCC299 (SEQ ID NO:18), Micromonas pusilla (SEQ ID NO:19), Sphagnum fallax (SEQ ID NO:20), Physcomitrella patens (SEQ ID NO:21), Arabidopsis thaliana (SEQ ID NO:22), Arabidopsis halleri (SEQ ID NO:23), Arabidopsis lyrata (SEQ ID NO:24), Helianthus annuus (SEQ ID NO:25), Vitis vinifera (SEQ ID NO:26), Amborella trichopoda (SEQ ID NO:27), Ricinus communis (SEQ ID NO:28), Solanum lycopersicum (SEQ ID NO:29), Solanum tuberosum (SEQ ID NO:30), Gossypium hirsutum (SEQ ID NO:31), Theobroma cacao (SEQ ID NO:32), Phaeolis vulgaris (SEQ ID NO:33), Glycine max (SEQ ID NO:34), Chenopodium quinoa (SEQ ID NO:35), Malus domesticus (SEQ ID NO:36), Zea mays (SEQ ID NO:37), Brassica rapa (SEQ ID NO:38), and Oryza sativa (SEQ ID NO:39). Also included as SGI1 polypeptides polypeptides having at least 50%, at least 55%, at least 60%, at least 65%, at least 70%, at least 75%, at least 80%, at least 85%, at least 90%, at least 95%, at least 96%, at least 97%, at least 98%, or at least 99% identity to any of the aforegoing, where the polypeptide has an RR domain and a myb domain, and the RR domain is N-terminal to the myb domain, where the SGI1 polypeptide is a naturally-occurring polypeptide or a variant thereof. In various embodiments, the SGI1 polypeptide is from a plant or algal species, i.e., is a naturally-occurring polypeptide of a plant or algal species. A gene encoding an SGI1 polypeptide as provided herein, for example a gene that is disrupted or whose expression is attenuated in a mutant as provided herein can be, in various embodiments, a naturally-occurring gene of a plant or algal species that encodes a polypeptide as disclosed herein.

In some embodiments, an SGI1 polypeptide as provided herein is an algal SGI1 polypeptide, for example, having the sequence of a naturally-occurring algal SGI1 polypeptide, where the algal polypeptide includes an RR domain and a myb domain, and the RR domain is N-terminal to the myb domain. The algal polypeptide can optionally have at least 50%, at least 55%, at least 60%, at least 65%, at least 70%, at least 75%, at least 80%, at least 85%, at least 90%, at least 95%, at least 96%, at least 97%, at least 98%, or at least 99% identity to any of the algal SGI1 polypeptides disclosed herein. In some embodiments, an SGI1 gene can be a gene encoding an algal SGI1 polypeptide, such as for example, a polypeptide having the sequence of a naturally-occurring algal SGI1 polypeptide. An SGI1 gene that encodes a polypeptide having the sequence of a naturally-occurring algal SGI polypeptide can be a gene having a naturally-occurring gene sequence of gene-encoding sequence, or can have a sequence that varies from the sequence of a naturally-occurring gene. In various embodiments, an SGI1 gene that is attenuated, mutated, or disrupted in a mutant photosynthetic organism as disclosed herein can be a gene that is identified through BLAST, for example, using one or more sequences disclosed herein as queries, and/or by HMM scanning, where the HMM is built from amino acid sequences, for example upon multiple alignment of at least six SGI1 polypeptides, where the amino acid sequences include an RR domain and a myb domain, where the RR domain is N-terminal to the myb domain, and where there is a linker sequence between the RR and myb domains that does not belong to either domain.

In some embodiments, an SGI1 polypeptide has the sequence of an algal SGI1 polypeptide or is a variant of a naturally-occurring algal SGI1 polypeptide having at least 85%, at least 90%, or at least 95% identity to an RR domain of a naturally-occurring algal SGI1 polypeptide and/or has has at least 50%, at least 55%, at least 60%, at least 65%, at least 70%, at least 75%, at least 80%, at least 85%, at least 90%, at least 95%, at least 96%, at least 97%, at least 98%, or at least 99% identity to any of SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49. In some embodiments, an SGI1 polypeptide has the sequence of an algal SGI1 polypeptide or is a variant of a naturally-occurring algal SGI1 polypeptide having at least 85%, at least 90%, or at least 95% identity to a myb domain of a naturally-occurring algal SGI1 polypeptide and/or has has at least 50%, at least 55%, at least 60%, at least 65%, at least 70%, at least 75%, at least 80%, at least 85%, at least 90%, at least 95%, at least 96%, at least 97%, at least 98%, or at least 99% identity to any of SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67.

In some embodiments, an SGI1 polypeptide has the sequence of an algal SGI1 polypeptide or is a variant of a naturally-occurring algal SGI1 polypeptide having at least 85%, at least 90%, or at least 95% identity to a naturally-occurring algal SGI1 polypeptide and/or has has at least 50%, at least 55%, at least 60%, at least 65%, at least 70%, at least 75%, at least 80%, at least 85%, at least 90%, at least 95%, at least 96%, at least 97%, at least 98%, or at least 99% identity to any of SEQ ID NO:3, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, or SEQ ID NO:19.

In some embodiments, an SGI polypeptide has the sequence of a plant SGI1 polypeptide or is a variant of a naturally-occurring plant SGI1 polypeptide having at least 85%, at least 90%, or at least 95% identity to a naturally-occurring algal SGI polypeptide and/or has has at least 50%, at least 55%, at least 60%, at least 65%, at least 70%, at least 75%, at least 80%, at least 85%, at least 90%, at least 95%, at least 96%, at least 97%, at least 98%, or at least 99% identity to an RR domain of, SEQ ID NO:50, SEQ ID NO:51, SEQ ID NO:52, SEQ ID NO:53, SEQ ID NO:54, SEQ ID NO:55, SEQ ID NO:56, or SEQ ID NO:57. In some embodiments, an SGI polypeptide has the sequence of a plant SGI1 polypeptide or is a variant of a naturally-occurring plant SGI1 polypeptide having at least 85%, at least 90%, or at least 95% identity to a naturally-occurring algal SGI polypeptide and/or has has at least 50%, at least 55%, at least 60%, at least 65%, at least 70%, at least 75%, at least 80%, at least 85%, at least 90%, at least 95%, at least 96%, at least 97%, at least 98%, or at least 99% identity to a myb domain of SEQ ID NO:68, SEQ ID NO:69, SEQ ID NO:70, SEQ ID NO:71, SEQ ID NO:72, SEQ ID NO:73, SEQ ID NO:74, or SEQ ID NO:75.

In some embodiments, an SGI polypeptide has the sequence of a plant SGI1 polypeptide or is a variant of a naturally-occurring plant SGI1 polypeptide having at least 85%, at least 90%, or at least 95% identity to a naturally-occurring algal SGI polypeptide and/or has has at least 50%, at least 55%, at least 60%, at least 65%, at least 70%, at least 75%, at least 80%, at least 85%, at least 90%, at least 95%, at least 96%, at least 97%, at least 98%, or at least 99% identity to any of SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, or SEQ ID NO:39.

### Mutant Strains

Mutants provided herein that have an attenuated SGI1 gene include photosynthetic organisms, such as eukaryotic photosynthetic organisms, including plants and algae, such as single celled eukaryotic algae (microalgae).

An algal strain having a mutated SGI1 gene which can be, in various examples, a strain genetically engineered to have attenuated expression of an SGI1 gene, can be any eukaryotic algal strain such as, for example, a species of any of the genera Achnanthes, Amphiprora, Amphora, Ankistrodesmus, Asteromonas, Boekelovia, Bolidomonas, Borodinella, Botrydium, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Desmodesmus, Dunaliella, Elipsoidon, Emiliania, Eremosphaera, Ernodesmius, Euglena, Eustigmatos, Franceia, Fragilaria, Fragilaropsis, Gloeothamnion, Haematococcus, Hantzschia, Heterosigma, Hymenomonas, Isochrysis, Lepocinclis, Micractinium, Monodus, Monoraphidium, Nannochloris, Nannochloropsis, Navicula, Neochloris, Nephrochloris, Nephroselmis, Nitzschia, Ochromonas, Oedogonium, Oocystis, Ostreococcus, Parachlorella, Parietochloris, Pascheria, Pavlova, Pelagomonas, Plurodactylum, Phagus, Picochlorum, Platymonas, Pleurochrysis, Pleurococcus, Prototheca, Pseudochlorella, Pseudoneochloris, Pseudostaurastrum, Pyramimonas, Pyrobotrys, Scenedesmus, Schizochlamydella, Skeletonema, Spyrogyra, Stichococcus, Tetrachlorella, Tetraselmis, Thalassiosira, Tribonema, Vaucheria, Viridiella, Vischeria, and Volvox.

For example, an alga having a mutation in an SGI1 gene as disclosed herein can be a species belonging to any of the phyla ochrophyta (including members of the bacillariophyceae, coscinodiscophyceae, fragilariophyceae, eustigmatophyceae, xanthophyceae, pelagophyceae, chrysophyceae, raphidophyceae, and synurophyceae), haptophyta (including members of the coccolithophyceae and pavlophyceae), and chlorophyta (including members of the trebouxiophyceae, chlorophyceae, nephrophyceae, pyramimonadophyceae, ulvophyceae, mamiellophyceae, and chlorodendrophyceae), as well as the charyophyta, euglenoids, and dinoflagellates.

In some embodiments of the present application, preferred microorganisms that may be mutated or genetically engineered include, but are not limited to, chlorophyte species such as Chlorella, Parachlorella, Pseudochlorella, Tetrachlorella, Auxenochlorella, Prototheca, Oocystis, Franceia, Micratinium, Picochlorum, Nannochloris, Schizochlamydella, Eremosphaera, Stichococcus, Botryococcus, Viridiella, Parietochloris Borodinella, Bracteacoccus, Neochloris, Monoraphidium, Desmodesmus, Scenedesmus, Ankistrodesmus, Carteria, Chlamydomonas, Chlorococcum, Chlorogonium, Volvox, Platymonas, Dunaliella, Haematococcus, Asteromonas, Pyrobotrys, Oedogonium, Nephroselmis, Pleurococcus, Pyramimonas, Pseudoneochloris, Ostreococcus, Tetraselmis, and Staurastrum.

In other examples, mutants can be engineered or isolated using a heterokont algal species such as a diatom species such as, for example, a species of any of the genera Amphora, Chaetoceros, Cyclotella, Fragilaria, Fragilaropsis, Hantzschia, Navicula, Nitzschia, Plurodactylum, or Thalassiosira. In further examples a mutant as disclosed herein is a species of the Eustigmatophyceae class, such as, for example, a species of Ellipsoidion, Eustigmatos, Vischeria, Monodus, Nannochloropsis, or Pseudostaurastrum. Other genera of the Ochrophyta that may be considered include, without limitation, Boldimonas, Botrydium, Baucheria, Tribonema, Monodus, Aureococcus, Bigeloweilla, Pelagomomas, Chrysosphaera, Ochromonas, Heterosigma, Nephrochloris, Boekelovia, Cricosphaera, Hymenomonas, Isochrysis, Pleurochrysis, and Pavlova.

In additional embodiments, organisms that may be mutated or engineered to include a mutated or attenuated SGI1 gene include plant species. A wide variety of plants and plant cell systems may be mutated or engineered to attenuate expression of an SGI gene, for example using nucleic acid constructs and various transformation methods known in the art (See Guerineau F., Methods Mol Biol. (1995) 49:1-32). In preferred embodiments, target plants and plant cells include, but are not limited to, those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot potato, sugar beets, yam), leafy vegetable crops (e.g., lettuce, spinach); flowering plants (e.g., petunia, rose, chrysanthemum), conifers and pine trees (e.g., pine fir, spruce), plants used in phytoremediation (e.g., heavy metal accumulating plants); oil crops (e.g., sunflower, rape seed) and plants used for experimental purposes (e.g., Arabidopsis). Thus, SGI1 mutants can be generated in a broad range of plants, such as for example with dicotyledonous plants belonging to the orders Magniolales, Miciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales. SGI1 mutants can also be generated in monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g those belonging to the orders Pinales, Ginkgoales, Cycadales, Araucariales, Cupressales and Gnetales, or with plants classified as mosses (Bryophyta), for example, Sphagnales and Funariales.

Exemplary and nonlimiting plant species that may have an attenuated SGI1 gene include Sphagnum fallax, Physcomitrella patens, Arabidopsis thaliana, Arabidopsis halleri, Arabidopsis lyrata, Helianthus annuus, Vitis vinifera, Amborella trichopoda, Ricinus communis, Solanum lycopersicum, Solanum tuberosum, Gossypium hirsutum, Theobroma cacao, Phaeolis vulgaris, Glycine max, Chenopodium quinoa, Malus domesticus, Zea mays, Brassica rapa, and Oryza sativa.

### Gene Attenuation

A mutant microorganism as provided herein having an attenuated SGI1 gene is a mutant generated by human intervention, for example, by classical mutagenesis or genetic engineering. For example, a mutant microorganism as provided herein can be a mutant generated by any feasible mutagenesis method, including but not limited to UV irradiation, gamma irradiation, or chemical mutagenesis, and screening for mutants having reduced chlorophylll and increased productivity, for example using the methods disclosed herein. For example, screening for reduced chlorophyll mutants can use fluorescence activated cell sorting (FACS), visual inspection, and/or extraction and measurement of chlorophyll absorption. Increased productivity can be assessed by growth of mutants and measurement of dry weight, wet weight, ash-free dry weight, or total organic carbon (TOC). Methods for generating mutants of photosynthetic organisms using classical mutagenesis and genetic engineering are well-known.

Examples of attenuated genes include, without limitation, genes that have one or more nucleotide substitutions that change the sequence of one or more amino acids resulting in reduced or abolished activity of the encoded protein; insertions or deletions (indels) in the gene that change the amino acid sequence of the protein by frameshifting and/or introducing stop codons; large insertions into the gene that result in frameshifting or truncation of the encoded polypeptide or instability of the RNA transcribed by the gene, large deletions that result in no functional protein being made; insertions, deletions, or point mutations in regions of the gene 5′ or 3′ of the coding region that affect transcription; and insertions, deletions, or point mutations in introns or at intron/exon junctiions that affect may affect transcription, splicing, or RNA stability. An attenuated gene can also be a gene whose transcript is targeted by, for example, RNAi, antisense RNA, ribozymes, or the like. A photosynthetic organism having an attenuated SGI1 gene is referred to herein as “an SGI1 mutant” regardless of the mechanism of attenuation of the SGI1 gene. SGI1 mutants can be isolated by methods disclosed herein, including mutagenesis and screening procedures disclosed herein as well as by genetic engineering.

Thus, a mutant can be a genetically engineered mutant, for example, a mutant in which an SGI1 gene has been targeted by homologous recombination for knock-out, knockdown, and/or gene replacement (for example with mutated form of the gene that may encode a polypeptide having reduced activity with respect to the wild type polypeptide). For example, a photosynthetic organism of interest may be engineered by site directed homologous recombination to insert a sequence into a genomic locus and thereby alter a gene and/or its expression, and/or to insert a promoter into a genetic locus of the host microorganism to affect the expression of a gene at the locus.

For example, gene knockout, gene knockdown, or gene replacement by homologous recombination can be by transformation of a nucleic acid (e.g., DNA) fragment that includes a sequence homologous to the region of the genome to be altered, where the homologous sequence is interrupted by a foreign sequence, typically a selectable marker gene that allows selection for the integrated construct. The genome-homologous flanking sequences on either side of the foreign sequence or mutated gene sequence can be for example, at least about 50, at least about 100, at least about 200, at least about 300, at least about 400, at least about 500, at least about 600, at least about 700, at least about 800, at least about 900, at least about 1,000, at least about 1,200, at least about 1,500, at least about 1,750, or at least about 2,000 (e.g., at least any of 50, 100, 200, 500, 1000, 1500 or 2000) nucleotides in length. A gene knockout or gene “knock in” construct in which a foreign sequence is flanked by target gene sequences, can be provided in a vector that can optionally be linearized, for example, outside of the region that is to undergo homologous recombination, or can be provided as a linear fragment that is not in the context of a vector, for example, the knock-out or knock-in construct can be an isolated or synthesized fragment, including but not limited to a PCR product. In some instances, a split marker system can be used to generate gene knock-outs by homologous recombination, where two DNA fragments can be introduced that can regenerate a selectable marker and disrupt the gene locus of interest via three crossover events (Jeong et al. (2007) FEMS Microbiol Lett 273: 157-163).

In one aspect this disclosure provides genetically modified organisms, e.g., microorganisms having one or more genetic modifications or mutations for attenuating expression of an SGI1 gene. As used herein “attenuating” or “altering” the expression and/or function” of a gene (e.g an SGI1 gene) means reducing or eliminating expression of the gene in any manner that reduces production, expression and/or function of the normally expressed fully functional protein. Means for attenuating a gene such as an SGI1 gene include, for example, homologous recombination constructs; CRISPR systems, including guide RNAs, Cas9 or other cas enzymes, e.g., Cpf1, Cms1, Csm1, or others, and optionally, donor fragments for insertion into the targeted site; RNAi constructs, including shRNAs, antisense RNA constructs; ribozyme constructs; TALENS, Zinc Finger nucleases; and meganucleases. For instance, in some embodiments, the gene can be disrupted by, for example, an insertion or gene replacement mediated by homologous recombination and/or by the activity of a double strand break inducing agent such as meganuclease (see, e.g., WO2012017329 (US20130164850 and US20160272980), zinc finger nuclease (Perez-Pinera et al. (2012) Curr. Opin. Chem. Biol. 16:268-277; WO2012017329 (US20130164850 and US20120324603), TALEN (WO2014/207043 (US20160130599); WO 2014/076571 (US20160272980)), or a cas protein (e.g., a Cas9 protein, Cpf1 effector protein, or Csm1 effector protein) of a CRISPR system (see e.g., U.S. Pat. Nos. 8,697,359; 8,795,965; 8,889,356; US 2016/0304893; US 2016/0090603; US2014/0068797; US 2016/0208243; US 2017/0233756). Other methods of disruption are known in the art and would be suitable here as would be understood by those of ordinary skill in the art.

In some embodiments, the mutant microorganism has one or more mutations that are present in, or one or more mutations affecting the expression of, an SGI1 gene. A recombinant microorganism engineered to have attenuated expression of an SGI1 gene can have a disrupted SGI1 gene that includes as least one insertion, mutation, or deletion that reduces or abolishes expression of the gene such that a fully functional SGI1 gene is not produced or is produced in lower amounts than is produced by a control microorganism that does not include a disrupted SGI1 gene. For instance, in some embodiments, one or more mutations (change, deletion, and/or insertion of one or more nucleotides) can be in the coding region of the gene or can be in an intron, 3′ UTR, 5′ UTR, or promoter region, e.g., within about 1 kb of the transcriptional start site, within about 2 kb of the transcriptional start site or within about 3 kb of the translational start site. In some embodiments, for example, a mutant microorganism having attenuated expression of a gene as disclosed herein can have one or more mutations, which can be one or more nucleobase changes and/or one or more nucleobase deletions and/or one or more nucleobase insertions, into the region of a gene 5′ of the transcriptional start site, such as, in non-limiting examples, within about 2 kb, within about 1.5 kb, within about 1 kb, or within about 0.5 kb of the known or putative transcriptional start site, or within about 3 kb, within about 2.5 kb, within about 2 kb, within about 1.5 kb, within about 1 kb, or within about 0.5 kb of the translational start site. As nonlimiting examples, a mutant gene can be a gene that has a mutation, insertion, or deletion within the promoter region that can either increase or decrease expression of the gene; can be a gene that has a deletion that results in production of a nonfunctional protein, truncated protein, dominant negative protein, or no protein; can be a gene that has one or more point mutations leading to a change in the amino acid of the encoded protein or results in aberrant splicing of the gene transcript, etc.

The CRISPR systems referred to herein, and reviewed by Hsu et al. (Cell 157:1262-1278, 2014) include, in addition to the cas nuclease polypeptide or complex, a targeting RNA, often denoted “crRNA”, that interacts with the genome target site by complementarity with a target site sequence, a trans-activating (“tracr”) RNA that complexes with the cas polypeptide and also includes a region that binds (by complementarity) the targeting crRNA. This disclosure contemplates the use of two RNA molecules (a “crRNA” and a “tracrRNA”) that can be co-transformed into a host strain (or expressed in a host strain) that expresses or is transfected with a cas protein for genome editing, or the use of a single guide RNA that includes a sequence complementary to a target sequence as well as a sequence that interacts with a cas protein. That is, in some strategies a CRISPR system as used herein can comprise two separate RNA molecules (RNA polynucleotides: a “tracr-RNA” and a “targeter-RNA” or “crRNA”, see below) and referred to herein as a “double-molecule DNA-targeting RNA” or a “two-molecule DNA-targeting RNA.” Alternatively, as illustrated in the examples, the DNA-targeting RNA can also include the trans-activating sequence for interaction with the cas protein (in addition to the target-homologous (“cr”) sequences), that is, the DNA-targeting RNA can be a single RNA molecule (single RNA polynucleotide) and is referred to herein as a “chimeric guide RNA,” a “single-guide RNA,” or an “sgRNA.” The terms “DNA-targeting RNA” and “gRNA” are inclusive, referring both to double-molecule DNA-targeting RNAs and to single-molecule DNA-targeting RNAs (i.e., sgRNAs). Both single-molecule guide RNAs and two RNA systems have been described in detail in the literature and for example, in U.S. Patent Application Publication No. US 2014/0068797, incorporated by reference herein in its entirety. Some embodiments of the methods and compositions presented herein include a guide RNA that has a sequence corresponding to a target sequence in an SGI1 gene. In some embodiments, the guide RNA is a chimeric guide. In other embodiments, the guide RNA does not include a tracr sequence. In some examples, both a crRNA for targeting the gene locus and a tracrRNA are employed in the cas-mediated mutagenesis. In some embodiments a cas protein is expressed in the target cell or organism, and in some alternative embodiments a cas protein may be introduced in to the cell, for example, as a ribonucleoprotein complex that includes a guide RNA, which may be a chimeric guide RNA or a crRNA and, in some embodiments, a tracrRNA. Any cas protein can be used in the methods herein, e.g., Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm1, Csm2, Csm3, Csm4, Csm5, Csm6, Cmr1, Cmr3, Cmr4, Cmr5, Cmr6, Cms1, Cpf1, Csb1, Csb2, Csb3, Csx17, Csx14, Csx10, Csx16, CsaX, Csx3, Csx1, Csx15, Csf1, Csf2, Csf3, Csf4, C2c1, C2c2, C2c3, and homologs thereof, or modified versions thereof. The cas protein can be a Cas9 protein, such as a Cas9 protein of Staphylococcus pyogenes, S. thermophilus, S. pneumonia, S. aureus, or Neisseria meningitidis, as nonlimiting examples. Also considered are the Cas9 proteins provided as SEQ ID NOs:1-256 and 795-1346 in U.S. Patent Application Publication No. US 2014/0068797, incorporated herein by reference in its entirety, and chimeric Cas9 proteins that may combine domains from more than one Cas9 protein, as well variants and mutants of identified Cas9 proteins. The RNA-guided nuclease can be, for example, a Cpf1 protein (see, for example, US 2016/0208243) or a Csm1 protein (see, for example, US 2017/0233756).

Cas nuclease activity cleaves target DNA to produce double strand breaks. These breaks are then repaired by the cell in one of two ways: non-homologous end joining or homology-directed repair. In non-homologous end joining (NHEJ), the double-strand breaks are repaired by direct ligation of the break ends to one another. In this case, no new nucleic acid material is inserted into the site, although some nucleic acid material may be lost, resulting in a deletion, or altered, often resulting in mutation. In homology-directed repair, a donor polynucleotide (sometimes referred to as a “donor DNA” or “editing DNA”) which may have homology to the cleaved target DNA sequence is used as a template for repair of the cleaved target DNA sequence, resulting in the transfer of genetic information from the donor polynucleotide to the target DNA. As such, new nucleic acid material may be inserted/copied into the site. The modifications of the target DNA due to NHEJ and/or homology-directed repair (for example using a donor DNA molecule) can lead to, for example, gene correction, gene replacement, gene tagging, transgene insertion, nucleotide deletion, gene disruption, gene mutation, etc.

In some instances, cleavage of DNA by a site-directed modifying polypeptide (e.g., a cas nuclease, zinc finger nuclease, meganuclease, or TALEN) may be used to delete nucleic acid material from a target DNA sequence by cleaving the target DNA sequence and allowing the cell to repair the sequence in the absence of an exogenously provided donor polynucleotide. Such NHEJ events can result in mutations (“mis-repair”) at the site of rejoining of the cleaved ends that can resulting in gene disruption.

Alternatively, if a DNA-targeting RNA is co-administered to cells that express a cas nuclease along with a donor DNA, the subject methods may be used to add, i.e., insert or replace, nucleic acid material to a target DNA sequence (e.g., “knock out” by insertional mutagenesis, or “knock in” a nucleic acid that encodes a protein (e.g., a selectable marker and/or any protein of interest), an siRNA, a miRNA, etc., to modify a nucleic acid sequence (e.g., introduce a mutation), and the like.

A donor DNA can in particular embodiments include a gene regulatory sequence (e.g., a promoter) that can, using CRISPR targeting, be inserted upstream of the coding regions of the gene and upstream of the presumed proximal promoter region of the gene, for example, at least about 50 bp, at least about 100 bp, at least about 120 bp, at least about 150 bp, at least about 200 bp, at least about 250 bp, at least about 300 bp, at least about 350 bp, at least about 400 bp, at least about 450 bp, or at least about 500 bp (e.g., at least 50, 100, 200, 300, 400 or 500 bp) upstream of the initiating ATG of the coding region of the SGI1 gene. The donor DNA can include a sequence, such as for example a selectable marker or any convenient sequence, that may be interfere with the native promoter. The additional sequence inserted upstream of the initiating ATG of the SGI1 open reading frame (e.g., in the 5′UTR or upstream of the transcriptional start site of the SGI! gene) can decrease or even eliminate expression of the endogenous SGI1 gene. Alternatively, or in addition, the native SGI1 gene can have its endogenous promoter wholly or partially replaced by a weaker or differently regulated promoter, or a non-promoter sequence.

In some examples, a nucleic acid molecule introduced into a host cell for generating a high efficiency genome editing cell line encodes a Cas9 enzyme that is mutated to with respect to the corresponding wild-type enzyme such that the mutated Cas9 enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (an enzyme that cleaves a single strand). Other examples of mutations that render Cas9 a nickase include, without limitation, H840A, N854A, and N863A. In some embodiments, a Cas9 nickase may be used in combination with guide sequence(s), e.g., two guide sequences, which target respectively sense and antisense strands of the DNA target. This combination allows both strands to be nicked and used to induce NHEJ. Two nickase targets (within close proximity but targeting different strands of the DNA) can be used to inducing mutagenic NHEJ. Such targeting of a locus using enzymes that cleave opposite strains at staggered positions can also reduce nontarget cleavage, as both strands must be accurately and specifically cleaved to achieve genome mutation. In additional examples, a mutant Cas9 enzyme that is impaired in its ability to cleave DNA can be expressed in the cell, where one or more guide RNAs that target a sequence upstream of the transcriptional or translational start site of the targeted gene are also introduced. In this case, the cas enzyme may bind the target sequence and block transcription of the targeted gene (Qi et al. (2013) Cell 152:1173-1183). This CRISPR interference of gene expression can be referred to as RNAi and is also described in detail in Larson et al. (2013) Nat. Protoc. 8: 2180-2196. In some cases, a cas polypeptide such as a Cas9 polypeptide is a fusion polypeptide, comprising, e.g.: i) a Cas9 polypeptide (which can optionally be variant Cas9 polypeptide as described above); and b) a covalently linked heterologous polypeptide (also referred to as a “fusion partner”). A heterologous nucleic acid sequence may be linked to another nucleic acid sequence (e.g., by genetic engineering) to generate a chimeric nucleotide sequence encoding a chimeric polypeptide. In some embodiments, a Cas9 fusion polypeptide is generated by fusing a Cas9 polypeptide with a heterologous sequence that provides for subcellular localization (i.e., the heterologous sequence is a subcellular localization sequence, e.g., a nuclear localization signal (NLS) for targeting to the nucleus; a mitochondrial localization signal for targeting to the mitochondria; a chloroplast localization signal for targeting to a chloroplast; an ER retention signal; and the like). In some embodiments, the heterologous sequence can provide a tag (i.e., the heterologous sequence is a detectable label) for ease of tracking and/or purification (e.g., a fluorescent protein, e.g., green fluorescent protein (GFP), YFP, RFP, CFP, mCherry, tdTomato, and the like; a hemagglutinin (HA) tag; a FLAG tag; a Myc tag; and the like).

Host cells can be genetically engineered (e.g., transduced or transformed or transfected) with, for example, a vector construct that can be, for example, a vector for homologous recombination that includes nucleic acid sequences homologous to a portion of an SGI1 gene locus of the host cell or to regions adjacent thereto, or can be an expression vector for the expression of any or a combination of: a cas protein (e.g., a Cas9 protein), a CRISPR chimeric guide RNA, a crRNA, and/or a tracrRNA, an RNAi construct (e.g., a shRNA), an antisense RNA, or a ribozyme. The vector can be, for example, in the form of a plasmid, a viral particle, a phage, etc. A vector for expression of a polypeptide or RNA for genome editing can also be designed for integration into the host, e.g., by homologous recombination. A vector containing a polynucleotide sequence as described herein, e.g., sequences having homology to host SGI1 gene sequences (including sequences that are upstream and downstream of the SGI1 polypeptide-encoding sequences), as well as, optionally, a selectable marker or reporter gene, can be employed to transform an appropriate host to cause attenuation of an SGI1 gene.

The recombinant microorganism in some examples can have reduced but not abolished expression of the SGI1 gene, and the recombinant microorganism can have an increase in lipid production of from about 25% to about 200% or more, for example. For example, the increase in lipid production can be between about 25% more to about 200% more, about 25% more to about 175% more, about 25% more to about 150% more, about 25% more to about 125% more, about 50% more to about 200% more, about 50% more to about 175% more, about 50% more to about 150% more, about 50% more to about 125% more, about 75% more to about 200% more, or about 75% more to about 175% more, about 75% more to about 150%, or about 75% more to about 125% more (e.g., 25-200% more) with respect to a control microorganism. A genetically modified phtosynthetic organism as provided herein can in some examples include a nucleic acid construct for attenuating the expression of an SGI1 gene. In some embodiments, genetically modified microorganism as provided herein can include a nucleic acid construct for attenuating the expression of an SGI1 polypeptide. For example, a host microorganism can include a construct for expressing an RNAi molecule, ribozyme, or antisense molecule that reduces expression of an SGI1 gene. In some examples, a recombinant microorganism as provided herein can include at least one introduced (exogenous or non-native) construct for reducing expression of an SGI1 gene.

In some examples, genetically engineered strains can be screened for expression of an SGI1 gene that is decreased with respect to a control cell that does not include a genetic modification for attenuating SGI1 gene expression, but not eliminated, using methods known in the art, such as, for example, RNA-Seq or reverse transcription-PCR (RT-PCR). A genetically engineered strain as provided herein can be engineered to include a construct for attenuating gene expression by reducing the amount, stability, or translatability of mRNA of a gene encoding an SGI1 polypeptide. For example, a microorganism such as an algal or heterokont strain can be transformed with an antisense RNA, RNAi, or ribozyme construct targeting an mRNA of an SGI1 gene using methods known in the art. For example, an antisense RNA construct that includes all or a portion of the transcribed region of a gene can be introduced into a microorganism to decrease gene expression (Shroda et al. (1999) The Plant Cell 11:1165-78; Ngiam et al. (2000) Appl. Environ. Microbiol. 66: 775-782; Ohnuma et al. (2009) Protoplasma 236: 107-112; Lavaud et al. (2012) PLoS One 7:e36806). Alternatively or in addition, an RNAi construct (for example, a construct encoding a short hairpin RNA) targeting a gene having a TPR domain can be introduced into a phohtosynthetic organism such as an alga or plant for reducing expression of the SGI1 gene (see, for example, Cerruti et al. (2011) Eukaryotic Cell (2011) 10: 1164-1172; Shroda et al. (2006) Curr. Genet. 49:69-84).

Ribozymes are RNA-protein complexes that cleave nucleic acids in a site-specific fashion. Ribozymes have specific catalytic domains that possess endonuclease activity. For example, U.S. Pat. No. 5,354,855 (incorporated herein in its entirety by reference) reports that certain ribozymes can act as endonucleases with a sequence specificity greater than that of known ribonucleases and approaching that of the DNA restriction enzymes. Catalytic RNA constructs (ribozymes) can be designed to base pair with an mRNA encoding a gene as provided herein to cleave the mRNA target. In some examples, ribozyme sequences can be integrated within an antisense RNA construct to mediate cleavage of the target. Various types of ribozymes can be considered, their design and use is known in the art and described, for example, in Haseloff et al. (1988) Nature 334:585-591. Ribozymes are targeted to a given sequence by virtue of annealing to a site by complimentary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence which is the cleavage site. For hammerhead ribozyme, the cleavage site is a dinucleotide sequence on the target RNA is a uracil (U) followed by either an adenine, cytosine or uracil (A, C or U) (Thompson et al., (1995) Nucl Acids Res 23:2250-68). The frequency of this dinucleotide occurring in any given RNA is statistically 3 out of 16. Therefore, for a given target messenger RNA of 1,000 bases, 187 dinucleotide cleavage sites are statistically possible.

The general design and optimization of ribozyme directed RNA cleavage activity has been discussed in detail (Haseloff and Gerlach (1988) Nature 334:585-591; Symons (1992) Ann Rev Biochem 61: 641-71; Chowrira et al. (1994) J Biol Chem 269:25856-64; Thompson et al. (1995) supra), all incorporated by reference in their entireties. Designing and testing ribozymes for efficient cleavage of a target RNA is a process well known to those skilled in the art. Examples of scientific methods for designing and testing ribozymes are described by Chowrira et al., (1994) supra and Lieber and Strauss (1995) Mol Cell Biol. 15: 540-51, each incorporated by reference. The identification of operative and preferred sequences for use in down regulating a given gene is a matter of preparing and testing a given sequence, and is a routinely practiced “screening” method known to those of skill in the art. The use of RNAi constructs is described in literature cited above as well as in US2005/0166289 and WO 2013/016267 (both of which are incorporated herein by reference), for example. A double stranded RNA with homology to the target gene is delivered to the cell or produced in the cell by expression of an RNAi construct, for example, an RNAi short hairpin (sh) construct. The construct can include a sequence that is identical to the target gene, or at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identical to a sequence of the target gene. The construct can have at least about 20, at least about 30, at least about 40, at least about 50, at least about 100, at least about 200, at least about 300, at least about 400, at least about 500, at least about 600, at least about 700, at least about 800, at least about 900, or at least about 1 kb of sequence (e.g., at least 20, 50, 100, 200, 400, 600, 800 or 1 kb of sequence) homologous to the target gene. Expression vectors can be engineered using promoters selected for continuous or inducible expression of an RNAi construct, such as a construct that produces an shRNA.

Promoters used in antisense, RNAi, or ribozyme constructs can be any that are functional in the host organism and that are suitable for the levels of expression required for reducing expression of the target gene to a desired amount. Promoters functional in algae and plants are known in the art and disclosed herein. The construct can be transformed into algae or plants using any feasible method, include any disclosed herein. A recombinant organism or microorganism transformed with a nucleic acid molecule for attenuating SGI1 gene expression, such as but not limited to an antisense, RNAi, or ribozyme construct, can have the properties of an SGI1 mutant as described herein, including, for example, reduced chlorophyll, increased photosynthetic efficiency, and increased productivity in culture, with respect to a host organism or microorganism that does not include the exogenous nucleic acid molecule that results in attenuated gene expression.

One skilled in the art will appreciate that a number of transformation methods can be used for genetic transformation of microorganisms and, therefore, can be deployed for the methods of the present invention. “Stable transformation” is intended to mean that the nucleic acid construct introduced into an organism integrates into the genome of the organism or is part of a stable episomal construct and is capable of being inherited by the progeny thereof. “Transient transformation” is intended to mean that a polynucleotide is introduced into the organism and does not integrate into the genome or otherwise become established and stably inherited by successive generations.

Genetic transformation can result in stable insertion and/or expression of transgenes, constructs from either the nucleus or the plastid, and in some cases can result in transient expression of transgenes. The transformation methods can also be used for the introduction of guide RNAs or editing DNAs. Genetic transformation of microalgae has been reported successful for more than 30 different strains of microalgae, which belong to at least ˜22 species of green, red, and brown algae, diatoms, euglenids, and dianoflagellates (see, e.g., Radakovits et al., Eukaryotic Cell, 2010; and Gong et al., J. Ind. Microbiol. Biotechnol., 2011). Non-limiting examples of such useful transformation methods include agitation of cells in the presence of glass beads or silicon carbide whiskers as reported by, for example, Dunahay, Biotechniques, 15(3):452-460, 1993; Kindle, Proc. Natl. Acad. Sci. U.S.A., 1990; Michael and Miller, Plant J., 13, 427-435, 1998. Electroporation techniques have been successfully used for genetic transformation of several microalgal species including Nannochloropsis sp. (see, e.g., Chen et al., J. Phycol., 44:768-76, 2008), Chlorella sp. (see, e.g., Chen et al., Curr. Genet., 39:365-370, 2001; Chow and Tung, Plant Cell Rep. Vol. 18, No. 9, 778-780, 1999), Chlamydomonas (Shimogawara et al., Genetics, 148: 1821-1828, 1998), and Dunaliella (Sun et al., Mol. Biotechnol. 30(3): 185-192, 2005), for example. Micro-projectile bombardment, also referred to as microparticle bombardment, gene gun transformation, or biolistic bombardment, has been used successfully for several algal species including, for example, diatoms species such as Phaeodactylum (Apt et al., Mol. Gen. Genet., 252:572-579, 1996), Cyclotella and Navicula (Dunahay et al., J. Phycol., 31:1004-1012, 1995), Cylindrotheca (Fischer et al., J. Phycol., 35:113-120, 1999), and Chaetoceros sp. (Miyagawa-Yamaguchi et al., Phycol. Res. 59: 113-119, 2011), as well as green algal species such as Chlorella (El-Sheekh, Biologia Plantarum, Vol. 42, No. 2: 209-216, 1999), and Volvox species (Jakobiak et al., Protist, 155:381-93, 2004). Additionally, Agrobacterium-mediated gene transfer techniques can also be useful for genetic transformation of microalgae, as has been reported by, for example, Kumar, Plant Sci., 166(3):731-738, 2004, and Cheney et al., J. Phycol., Vol. 37, Suppl. 11, 2001. Conjugation with bacterial species has also been employed for transfer of genes and constructs to algae, as disclosed for example in US 2016/0244770, incorporated herein by reference.

A transformation vector or construct as described herein will typically comprise a marker gene that confers a selectable or scorable phenotype on target host cells, e.g., algal cells or may be co-transformed with a construct that includes a marker. A number of selectable markers have been successfully developed for efficient isolation of genetic transformants of algae. Common selectable markers include antibiotic resistance, fluorescent markers, and biochemical markers. Several different antibiotic resistance genes have been used successfully for selection of microalgal transformants, including blastocidin, bleomycin (see, for example, Apt et al., 1996, supra; Fischer et al., 1999, supra; Fuhrmann et al., Plant J., 19, 353-61, 1999, Lumbreras et al., Plant J., 14(4):441-447, 1998; Zaslayskaia et al., J. Phycol., 36:379-386, 2000), spectinomycin (Cerutti et al., Genetics, 145: 97-110, 1997; Doetsch et al., Curr. Genet., 39, 49-60, 2001; Fargo, Mol. Cell. Biol., 19:6980-90, 1999), streptomycin (Berthold et al., Protist, 153:401-412, 2002), paromomycin (Jakobiak et al., Protist, supra.; Sizova et al., Gene, 277:221-229, 2001), nourseothricin (Zaslayskaia et al., 2000, supra), G418 (Dunahay et al., 1995, supra; Poulsen and Kroger, FEBS Lett., 272:3413-3423, 2005, Zaslayskaia et al., 2000, supra), hygromycin (Berthold et al., 2002, supra), chloramphenicol (Poulsen and Kroger, 2005, supra), and many others. Additional selectable markers for use in microalgae such as Chlamydomonas can be markers that provide resistance to kanamycin and amikacin resistance (Bateman, Mol. Gen. Genet. 263:404-10, 2000), zeomycin and phleomycin (e.g., ZEOCIN™ pheomycin D1) resistance (Stevens, Mol. Gen. Genet. 251:23-30, 1996), and paramomycin and neomycin resistance (Sizova et al., 2001, supra). Other fluorescent or chromogenic markers that have been used include luciferase (Falciatore et al., J. Mar. Biotechnol., 1: 239-251, 1999; Fuhrmann et al., Plant Mol. Biol., 2004; Jarvis and Brown, Curr. Genet., 19: 317-322, 1991), β-glucuronidase (Chen et al., 2001, supra; Cheney et al., 2001, supra; Chow and Tung, 1999, supra; El-Sheekh, 1999, supra; Falciatore et al., 1999, supra; Kubler et al., J. Mar. Biotechnol., 1:165-169, 1994), β-galactosidase (Gan et al., J. Appl. Phycol., 15:345-349, 2003; Jiang et al., Plant Cell Rep., 21:1211-1216, 2003; Qin et al., High Technol. Lett., 13:87-89, 2003), and green fluorescent protein (GFP) (Cheney et al., 2001, supra; Ender et al., Plant Cell, 2002, Franklin et al., Plant J., 2002; 56, 148, 210).

One skilled in the art will readily appreciate that a variety of known promoter sequences can be usefully deployed for transformation systems of microalgal species in accordance with the present invention. For example, the promoters commonly used to drive transgene expression in microalgae include various versions of the of cauliflower mosaic virus promoter 35S (CaMV35S), which has been used in both dinoflagellates and chlorophyta (Chow et al, Plant Cell Rep., 18:778-780, 1999; Jarvis and Brown, Curr. Genet., 317-321, 1991; Lohuis and Miller, Plant J., 13:427-435, 1998). The SV40 promoter from simian virus has also reported to be active in several algae (Gan et al., J. Appl. Phycol., 151 345-349, 2003; Qin et al., Hydrobiologia 398-399, 469-472, 1999). The promoters of RBCS2 (ribulose bisphosphate carboxylase, small subunit) (Fuhrmann et al., Plant J., 19:353-361, 1999) and PsaD (abundant protein of photosystem I complex; Fischer and Rochaix, FEBS Lett. 581:5555-5560, 2001) from Chlamydomonas can also be useful. The fusion promoters of HSP70A/RBCS2 and HSP70A/β2TUB (tubulin) (Schroda et al., Plant J., 21:121-131, 2000) can also be useful for an improved expression of transgenes, in which HSP70A promoter may serve as a transcriptional activator when placed upstream of other promoters. High-level expression of a gene of interest can also be achieved in, for example diatoms species, under the control of a promoter of an fcp gene encoding a diatom fucoxanthin-chlorophyll a/b binding protein (Falciatore et al., Mar. Biotechnol., 1:239-251, 1999; Zaslayskaia et al., J. Phycol. 36:379-386, 2000) or the vcp gene encoding a eustigmatophyte violaxanthin-chlorophyll a/b binding protein (see U.S. Pat. No. 8,318,482, incorporated by reference herein). If so desired, inducible promoters can provide rapid and tightly controlled expression of genes in transgenic microalgae. For example, promoter regions of the NR genes encoding nitrate reductase can be used as such inducible promoters. The NR promoter activity is typically suppressed by ammonium and induced when ammonium is replaced by nitrate (Poulsen and Kroger, FEBS Lett 272:3413-3423, 2005), thus gene expression can be switched off or on when microalgal cells are grown in the presence of ammonium/nitrate. An ammonium-repressible Nannochloropsis promoter referred to as the “Ammonia repressible Nitrite/Sulfite Reductase” promoter is disclosed in US 2017/0073695, incorporated herein by reference. Additional algal promoters that can find use in the constructs and transformation systems provided herein include those disclosed in U.S. Pat. Nos. 8,835,419; 8,883,993; U.S. Patent Appl. Pub. No. US 2013/0023035; U.S. Patent Application Pub. No. US 2013/0323780; U.S. Patent Application Pub. No. US 2014/0363892, U.S. Patent Application Pub. No. US 2017/0152520, and U.S. Patent Application Pub. No. US 2017/0114107, all incorporated herein by reference in their entireties.

Host cells or organisms can be either untransformed cells or organisms or can be cells or organisms that are already transfected with at least one exogenous nucleic acid molecule. For example, an algal or plant host cell that is engineered to have attenuated expression of an SGI1 gene can further include one or more transgenes that may confer or contribute to any desirable trait, such as, but not limited to, increased production of biomolecules of interest, such as one or more proteins, pigments, alcohols, or lipids.

Transformation of algal cells by microbombardment is described, for example, in U.S. Pat. No. 8,883,993 and US Patent Application publication no. US 2017/0130238, both of which are incorporated by reference in their entireties. Bombardment of cells using the BioRad Helios® Gene Gun is also described in the Helios Gene Gun System Instruction Manual, M1652411 available at bio-rad.com/en-us/product/helios-gene-gun-system?tab=Documents).

In various aspects, gene-attenuating mutations are generated in a plant. The methods include, for example introducing CRISPR system as described herein to target one or more plant SGI1 genes. A wide variety of plants and plant cell systems may be engineered for the desired physiological and agronomic characteristics described herein using the nucleic acid constructs of the present disclosure and the various transformation methods known in the art (See Guerineau F., Methods Mol Biol. (1995) 49:1-32). In preferred embodiments, target plants and plant cells for engineering include, but are not limited to, those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot potato, sugar beets, yam), leafy vegetable crops (e.g., lettuce, spinach); flowering plants (e.g., petunia, rose, chrysanthemum), conifers and pine trees (e.g., pine fir, spruce), plants used in phytoremediation (e.g., heavy metal accumulating plants); oil crops (e.g., sunflower, rape seed) and plants used for experimental purposes (e.g., Arabidopsis). Thus, SGI1 mutants can be generated in a broad range of plants, such as for example with dicotyledonous plants belonging to the orders Magniolales, Miciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapens ales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales.

SGI1 mutants can be also be generated in monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g those belonging to the orders Pinales, Ginkgoales, Cycadales, Araucariales, Cupressales and Gnetales.

Alternatively or in addition to any of the embodiments set forth above, included herein are the following embodiments.

Embodiment 1 is a mutant photosynthetic organism having an attenuated gene encoding an SGI1 polypeptide, wherein the the photosynthetic organism produces more biomass or more of at least one bioproduct than a control photosynthetic organism grown or cultured under the substantially the same conditions, optionally wherein one or more of the following are satisfied:
a) the mutant photosynthetic organism has reduced chlorophyll with respect to the control photosynthetic microorganism;
b) the mutant photosynthetic organism has an increased chlorophyll a:b ratio with respect to the control photosynthetic organism;
c) the mutant photosynthetic organism has a reduced photosystem II (PSII) antenna size with respect to the control photosynthetic organism;
d) the mutant photosynthetic organism according to claim 1, wherein the mutant photosynthetic organism has a higher ¹⁴C Pmax with respect to the control photosynthetic organism;
e) the mutant photosynthetic organism according to claim 1, wherein the mutant photosynthetic organism has a higher Fv/Fm than the control organism;
f) the mutant photosynthetic organism has a higher protein content than the control organism; and
g) the the mutant photosynthetic organism has a higher rubisco activase content than the control organism.

Embodiment 2 is a mutant photosynthetic organism according to embodiment 1, wherein the mutant photosynthetic organism produces at least 10%, at least 15%, at least 20%, at least 25%, at least 30%, at least 35%, at least 40%, at least 45%, at least 50%, at least 60%, at least 70%, at least 80%, at least 90% or at least 100% more biomass or at least 10%, at least 15%, at least 20%, at least 25%, at least 30%, at least 35%, at least 40%, at least 45%, at least 50%, at least 60%, at least 70%, at least 80%, at least 90% or at least 100% more of at least one bioproduct than a control photosynthetic organism grown or cultured under the substantially the same conditions, optionally wherein the bioproduct is one or more lipids, one or more carbohydrates, one or more proteins or peptides, one or more nucleic acids, one or more sugars, one or more alcohols, one or more amino acids, one or more nucleotides, one or more antioxidants, one or more pigments or colorants, one or more vitamins, one or more terpenoids, or one or more polymers.

Embodiment 3 is a mutant photosynthetic organism according to embodiment 1 or embodiment 2, wherein the SGI1 gene encodes an SGI polypeptide having a myb domain C-terminal to a Response Regulator receiver domain, optionally wherein the SGI1 polypeptide:
a) comprises an amino acid sequence that encodes a Response Regulator receiver (RR) domain having least 50%, at least 55%, at least 60%, at least 65%, at least 70%, at least 75%, at least 80%, at least 85%, at least 90%, or at least 95% identity to any of SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, or SEQ ID NO:57; and/or
b) comprises an amino acid sequence that encodes a myb domain having least 50%, at least 55%, at least 60%, at least 65%, at least 70%, at least 75%, at least 80%, at least 85%, at least 90%, or at least 95% identity to any of SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, or SEQ ID NO:75.

Embodiment 4 is a mutant photosynthetic organism according to any of embodiments 1-3, wherein the SGI1 gene is mutated, optionally wherein:
a) the SGI1 gene comprises an indel that inactivates the gene;
b) the SGI1 gene comprises a mutation that changes at least one amino acid of the SGI1 polypeptide encoded by the gene;
c) the SGI1 gene comprises an insertion of an introduced DNA fragment that inactivates the gene;
d) the SGI1 gene comprises an insertion of an introduced DNA fragment in the 5′ or 3′ untranslated region of the gene that reduces expression of the gene; and/or
e) the SGI1 gene comprises an indel in the 5′ or 3′ untranslated region of the gene that reduces expression of the gene.

Embodiment 5 is a mutant photosynthetic organism according to any of embodiments 1-3, wherein the mutant photosynthetic organism comprises an RNAi construct, an antisense construct, or a ribozyme construct that targets the SGI1 gene transcript.

Embodiment 6 is a mutant photosynthetic organism according to any of embodiments 1-5, wherein the mutant photosynthetic organism is a plant, optionally a monocot, dicot, or moss.

Embodiment 7 is a mutant photosynthetic organism according to any of embodiments 1-5, wherein the mutant photosynthetic organism is an alga, optionally a microalga, optionally a Chlorophyte or Charyophyte alga.

Embodiment 7 is a mutant photosynthetic organism according to embodiment 7, wherein the mutant photosynthetic microorganism is a Chlorophyte alga of the Chlorophyceace, the Chlorodendrophyceae, the Prasinophyceace, or the Trebouxiophyceae class.

Embodiment 8 is a biomass comprising a mutant photosynthetic organism of any of the above embodimets.

Embodiment 9 is a method of producing biomass or a bioproduct comprising cultivating any of the mutant photosynthetic organisms of embodiments 1-7 and isolating biomass.

### EXAMPLES

### Media

PM119 is a nutrient replete medium that includes: 35 ppt Instant Ocean Salts (Aquatic Eco Systems; Apopka, FL), 5× Guillard's F/2 marine water enrichment solution (50× stock from Sigma-Aldrich, St. Louis, MO, cat. No. G0154; final concentrations of components in media: 4.413 mM Sodium nitrate; 0.16 mM Sodium phosphate monobasic; 0.103 μM Biotin; 0.240 μM Cobalt chloride·6H₂O; 0.200 μM Cupric sulfate 5H₂O; 0.0585 mM Disodium EDTA·2H₂O; 4.54 μM Manganese chloride·4H₂O; 0.124 μM Sodium molybdate·2H₂O; 1.48 μM Thiamine·HCl; 0.0185 μM Vitamin B₁₂; 0.382 μM Zinc sulfate·7H₂O).

PM074 is a nutrient replete medium that is made by adding 1.3 ml PROLINE® F/2 Algae Feed Part A (Aquatic Eco-Systems) and 1.3 ml PROLINE® F/2 Algae Feed Part B (Aquatic Eco-Systems) to a final volume of 1 liter of a solution of Instant Ocean salts (35 g/L) (Aquatic Eco Systems, Apopka, FL). Proline A and Proline B together include 8.8 mM NaNO₃, 0.361 mM NaH₂PO₄·H₂O, 10× F/2 Trace metals, and 10× F/2 Vitamins (Guillard (1975) Culture of phytoplankton for feeding marine invertebrates. in “Culture of Marine Invertebrate Animals.” (eds: Smith W. L. and Chanley M. H.) Plenum Press, New York, USA. pp 26-60).

PM147 is a nutrient replete medium that is based on PM074 but has 50% of the salt. It is made by adding 1.3 ml PROLINE® F/2 Algae Feed Part A (Aquatic Eco-Systems) and 1.3 ml PROLINE® F/2 Algae Feed Part B (Aquatic Eco-Systems) to a final volume of 1 liter of a solution of Instant Ocean salts (17.5 g/L) (Aquatic Eco Systems, Apopka, FL).

PM153 is a nutrient replete medium that is based on PM074 but includes urea instead of nitrate as the nitrogen source. It is made by adding 1.3 ml PROLINE® F/2 Algae Feed Part A (Aquatic Eco-Systems) and 1.3 ml ‘Solution C’ to a final volume of 1 liter of a solution of Instant Ocean salts (17.5 g/L) (Aquatic Eco Systems, Apopka, FL), and then adding 4 ml if 1.1 M filter-sterilized urea. Solution C is 38.75 g/L NaH₂PO₄ H₂O, 758 mg/L Thiamine HCl, 3.88 mg/L vitamin B12, and 3.84 mg/L biotin.

### Example 1

### UV Mutagenesis of a Parachlorella Strain

To isolate mutants from a chlorophyte, or green algal species, cells of Parachlorella strain WT-1185 were mutagenized with UV and selected based on low chlorophyll fluorescence after low light acclimation. The Parachlorella strain used for mutagenesis, WT-1185, was isolated from a marine environment. Parachlorella WT-1185 cells were grown to mid-log phase and then diluted to 1×10⁶ cells/mL with growth medium PM119. The cell suspensions were transferred by pipet to a 100 mm Petri dish and placed within a STRATALINKER® 2400 UV crosslinker (Agilent Technologies, Santa Clara, CA) with the plate lid removed. UV irradiation was carried out with 10,000, 25,000, and 50,000 μJ/cm². After irradiation, cell suspensions were pipetted into a shake flask wrapped in foil to prevent light exposure for twenty-four hours during recovery.

### Example 2

### Screens for Low Chlorophyll Parachlorella Sp. Strain WT-1185 Mutants

Following mutagenesis and recovery as described in Example 1, cells from pale-colored colonies were selected and allowed to grow from between one and five days in low (100 μmol photons m⁻² sec⁻¹) light, after which they were sorted by flow cytometry using a BD FACSAria II flow cytometer (BD Biosciences, San Jose, CA) to select cells having low chlorophyll fluorescence. In general, the portion of cells with the lowest approximately 0.5 to 2% of chlorophyll fluorescence compared to the total population of cells was selected. Further primary screening of antenna-reduced lines isolated through flow cytometry was conducted through the selection of pale green or yellow colonies visually after sorted cells were plated. In order to screen putative antenna-reduced lines from other reduced pigment mutants and false positives, selected colonies were subjected to a medium-throughput secondary cultivation screen to acclimate the isolates to low light conditions prior to photo-physiological measurements. Chlorophyll fluorescence was monitored during low light acclimation to select colonies that retained the reduced chlorophyll fluorescence characteristic of the high light acclimated state. Clones that were selected demonstrated only small increases in chlorophyll (relative to wild type cells) when transferred from high to low light.

Semicontinuous culture assays in constant high light (approximately 1,700 μmol photons m⁻² sec⁻¹) using 165 ml cultures in 75 cm² tissue culture flasks were performed to identify strains having increased productivity (increased rate of biomass production, measured as TOC accumulation) with respect to the wild type progenitor strain WT-1185. Two 75 cm² flasks were inoculated with seed culture of a given mutant strain. The flasks had stoppers having tubing connected with syringe filters for delivering CO₂-enriched air (1% CO₂) that was bubbled through the cultures. The flasks were aligned with the width (narrowest dimension) against an LED light bank. The depth of the cultures (the distance from the wall of the flask nearest the light source to the wall at back of the flask) was approximately 8.0 cm. The cultures were diluted daily at the beginning of the light period by removing 65% of the culture volume and replacing it with fresh PM119 media diluted to adjust for the increase in salinity due to evaporation occurring in the cultures (212 ml di H₂O to 1 L PM119 medium). Samples for TOC analysis were taken from the culture removed for the dilution. Two isolates, NE-7843 and NE-13380, that were identified in this assay as having increased productivity were analyzed further.

### Example 3

### Semi-Continuous Productivity Assays of Algal Mutants NE-7843 and NE-13380

Among the Parachlorella strains that were found to have reduced chlorophyll under low light conditions were two isolates that were analyzed for increased productivity: mutants NE-7843 and NE-13380. In the productivity assay, photoautotrophic cultures of the mutants were grown over several days in constant light semi-continuous mode (CL-SCPA) with culture samples removed daily for biomass determination. The light was kept at a constant 1900-2000 μmol photons m⁻² sec⁻¹ for 24 hours per day. In this assay PM119 culture medium in a 225 cm² flask was inoculated with seed culture of a given mutant strain. Three cultures were initiated per strain. The flasks included stir bars and had stoppers having tubing connected with syringe filters for delivering CO₂ enriched air (1% CO₂) that was bubbled through the cultures. The flasks were aligned with the width (narrowest dimension) against an LED light bank. The “depth” dimension of the flasks, extending back from the light source, was 13.7 cm. Taking into account the positioning of the flasks the farthest distance of the cells in the flasks from the surface of the light source was approximately 15.5 cm. The cultures were diluted daily by removing 65% of the culture volume and replacing it with fresh PM119 culture medium diluted to adjust for the increase in salinity due to evaporation occurring in the cultures. Samples for TOC analysis were taken from the culture removed for the dilution. The semi-continuous productivity assay was run for 12 days once the cultures had reached steady state.

Productivity for the assay was assessed by measuring total organic carbon (TOC) from the samples that were removed daily. Total organic carbon (TOC) was determined by diluting 2 mL of cell culture to a total volume of 20 mL with DI water. Three injections per measurement were injected into a Shimadzu TOC-Vcsj Analyzer for determination of Total Carbon (TC) and Total Inorganic Carbon (TIC). The combustion furnace was set to 720° C., and TOC was determined by subtracting TIC from TC. The 4 point calibration range was from 2 ppm to 200 ppm corresponding to 20-2000 ppm for non-diluted cultures with a correlation coefficient of r²>0.999.

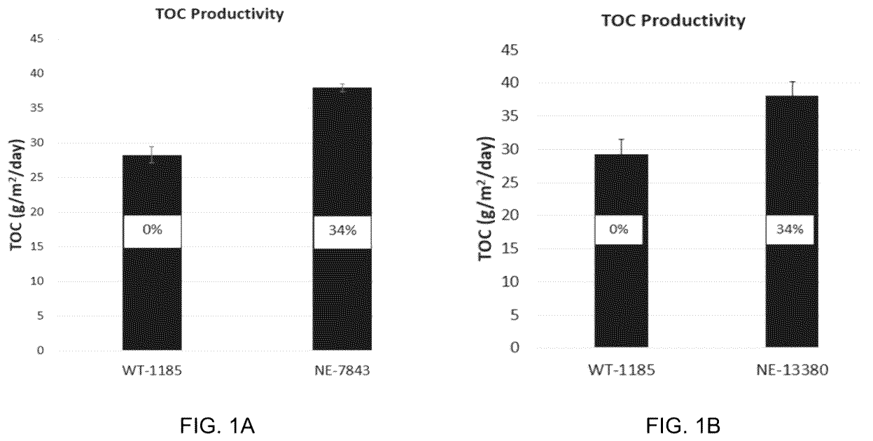

Exemplary results of the small-scale productivity assays are provided in FIG. 1A for mutant NE-7843 and FIG. 1B for mutant NE-13380, where the average daily productivity of duplicate cultures over 12 days of the culturing is shown. FIG. 1A and FIG. 1B demonstrate that mutants NE-7843 and NE-13380, respectively, had higher productivity in this CLSCPA assay, each demonstrating an average 34% increase over wild type in TOC over the course of the 12 day assay.

### Example 4

### Genotyping of Parachlorella Lihla Mutants

The genomes of NE-13380 and NE-7843 were sequenced to identify mutations such as single nucleotide polymorphisms (SNPs) or insertion/deletion mutations (indels), any of which could form the genetic basis for the phenotypes observed in these two strains. SNPs were identified in the genome of NE-13380 that caused mutations in the coding regions of four different genes (first four rows of Table 1).

TABLE 1
SNPs in NE-13380 Genome

| Mutated | | | Coding Sequence |
|---|---|---|---|
| Gene(s) | Description | SNP Type | Modification |
| T-0699 | Winged helix DNA-binding | Missense variant | Trp13Arg |
| | domain | | |
| | chain C domain 1 | | |
| T-4339 | conserved predicted protein | Stop-gained | Arg575Stop |
| T-0841, | Flagellar-associated protein | Missense variant | Ser1019Leu |
| T-1004 | calcium transporting ATPase | | |
| T-2261 | CheY-like protein | Splice acceptor variant | potential intron retention |
| | | & intron variant | |
| T-0898, | Conserved predicted protein | intergenic region | none |
| T-0899, | Dynein light chain 4 | | |
| T-0900 | axonemal | | |
| T-2627 | RNA helicase ATP-dependent | intron variant | none |
| | SK12/DOB1 protein isoform 1 | | |
| T-9629 | ADP-ribosylation factor | Upstream gene | none |
| | GTPase−activating protein | variant, intron variant | |
| | AGD5 isoform 1 and | | |
| | conserved predicted protein | | |

While this analysis of NE-13380 mutations was underway, analysis of the NE-7843 genome revealed that this strain also harbored distinct SNPs (Table 2), including an SNP in the T-2261 gene (gene sequence provided as SEQ ID NO:1; coding sequence provided as SEQ ID NO:2 encoding the protein sequence of SEQ ID NO:3), a gene also mutated in NE-13380 (row 4 of Table 1)—providing strong evidence that mutations in the T-2261 gene were the cause of the highly desirable high productivity phenotype of these strains.

TABLE 2
SNPs in NE-7843 Genome

| Mutated | | | Coding |
|---|---|---|---|
| | | | Sequence |
| Gene(s) | Description | SNP Type | Modification |
| T-0936 | Zn finger Ran-binding family | Stop-gained | *329Lys |
| | protein | | |
| T-4448 | Cytochrome P450 CYP | Missense variant | Leu508Cys |
| | 711clan | | |
| T-2261 | Type b response regulator | Missense variant | Leu250Pro |
| | Sensor histidine kinase/Che Y | | |
| T-3935 | Pyruvate−flavodoxin | Missense variant | Lys577Glu |
| | oxidoreductase | | |
| T-8682 | Dynein light chain LC8 type 2 | Missense variant | Tyr77His |
| T-4805 | Sm-like ribonucleoproteins | Missense variant | Asp32Asn |
| T-5398 | Aldolase class I | Missense variant | Gly26Cys |

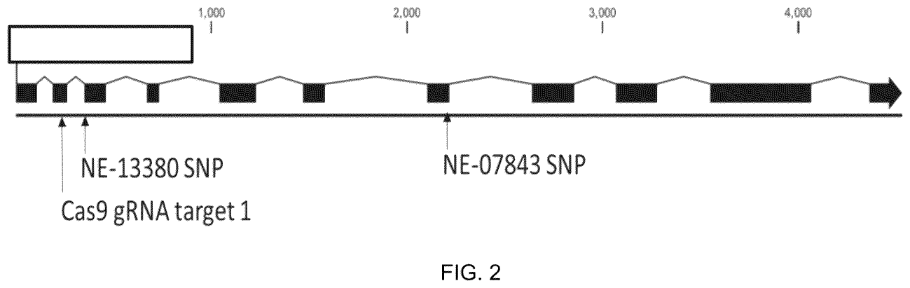

FIG. 2 shows the location of the nutations in the Parachlorella T-2661 gene having a response regulator CheY-like domain.

### Example 5

### Generating Targeted Mutations in the Parachlorella T-2661 “Response Regulator Chey-Like Protein” Gene

To definitively demonstrate that mutation of the T-2261 gene resulted in the high productivity phenotype, three strains were generated in which the T-2261 gene was knocked out by Cas9-mediated insertional mutagenesis with a guide RNA targeting the 3′end of the second exon of the T-2261 gene (FIG. 2). The single guide RNA (sg RNA) (SEQ ID NO:5) included the target sequence of SEQ ID NO:4 (i.e., the CRISPR sequence or sequence corresponding to the target sequence of the T-2261 gene), and was produced by annealing two complementary DNA oligomers that incorporated the sequence of the T7 promoter to generate a double-stranded template for in vitro transciption. In vitro transcription reactions were performed using the MEGAshortscript™ T7 Kit (Life Technologies cat #AM1354M; Carlsbad, CA) according to the manufacturer's protocol. The resulting RNA was purified using Zymo-Spin™ V-E columns (Zymo Research; Irvine, CA; cat #C1024-25) according to the manufacturer's protocol.

Purified guide RNA was transformed into cells of the Parachlorella Cas9-expressing Editor cell line GE-15699. The Cas9-expressing Editor line was produced by transforming wild type Parachlorella strain WT-1185 with a construct that included: 1) a Cas9 expression cassette which contained an engineered Cas9 gene codon optimized for Parachlorella and containing introns from Parachlorella, that also included an N-terminal FLAG tag, nuclear localization signal, and peptide linker (SEQ ID NO:76) operably linked to the Parachlorella RPS17 promoter (SEQ ID NO:77) and the Parachlorella RPS17 terminator (SEQ ID NO:78); 2) a selectable marker expression cassette, which contained the blasticidin resistance gene from Aspergillus terreus codon optimized for Parachlorella and containing Parachlorella introns (SEQ ID NO:79), operably linked to the Parachlorella RPS4 promoter (SEQ ID NO:80) and the Parachlorella RPS4 terminator (SEQ ID NO:81); and 3) a GFP reporter expression cassette, which contained the TurboGFP gene (Evrogen, Moscow, Russia) (SEQ ID NO:82), driven by the Parachlorella ACP1 promoter (SEQ ID NO:83) and terminated by the Parachlorella ACP1 terminator (SEQ ID:84). (See for example copending commonly-owned US Patent application publication US 2016/0304896 as well as US patent application publication US 2017/0073695 and US patent application publication US 2017/0152520, all of which are incorporated herein by reference in their entireties.)

The guide RNA (SEQ ID NO:5) was transformed in Editor line GE-15699 along with a DNA donor fragment that included a bleomycin resistance “BleR” gene codon-optimized for Parachlorella and containing Parachlorella introns (SEQ ID NO:6) operably linked to the Parachlorella RPS4 promoter (SEQ ID NO:7) and the Parachlorella RPS4 terminator (SEQ ID NO:8). Transformation was by electroporation, essentially as described in US 2016/0304896. Following electroporation, cells were plated on agar medium containing 250 μg/ml zeocin to select for transformants that incorporated the BleR cassette. Transformants were screened by colony PCR using primers designed to amplify across the native targeted locus and three strains were selected that demonstrated the insertion of the BleR cassette into the T-2661 gene locus: GE-16391, GE-16392, and GE-16393.

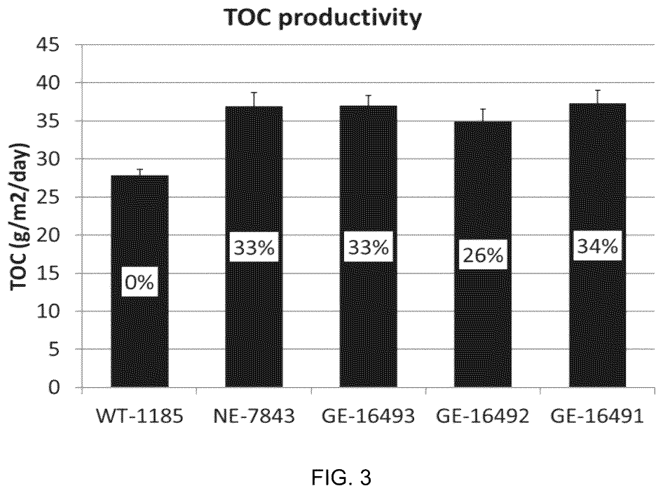

Cas9-engineered mutants GE-16391, GE-16392, and GE-16393, and classically-derived mutant NE-7843 were assayed under continuous light in a semi-continuous dilution productivity assay (SCPA) under conditions described in Example 3 (the CL2000 assay: the light was kept at a constant 1900-2000 μmol photons M⁻² sec⁻¹ for 24 hours per day and the culture was diluted back by 65% daily). The Cas9-engineered mutants were found to exhibit TOC productivity increases over wild type strains at a magnitude essentially identical to that observed in the original strains isolated from classical mutagenesis (FIG. 3), with the GE-16392 demonstrating about a 26% increase in biomass productivity and the GE-16391 and GE-16393 mutants demonstrating biomass productivity increases of 33% and 34%. From this it was concluded that knockout or severe reduction of T-2661 expression or function in Parachlorella WT1185 can lead to 25-35% increases in biomass productivity under continuous high light conditions. Because of its influence on productivity, the T-2661 gene was named SGI1, or Significant Growth Improvement 1.

### Example 6

### Parachlorella SGI1 (T-2661) Gene and Orthologs in Other Photosynthetic Organisms

The Parachlorella SGI1 gene (SEQ ID NO:1, coding sequence provided as SEQ ID NO:2) was found to encode a polypeptide (SEQ ID NO:3) that includes two major functional domains, both occurring in the N-terminal half of the 619 amino acid protein. The presence of a Response Regulator receiver or “RR” domain (Pfam PF00072), extending from approximately amino acid 36 to amino acid 148 of the Parachlorella SGI1 polypeptide (SEQ ID NO:3), is responsible for the bioinformatic annotation of SGI1 as a CheY-like polypeptide (see Tables 1 and 2). This RR domain is also characterized as a “signal receiver domain”, cd00156, in the conseved domain database (CDD), extending approximately from amino acid 37 through amino acid 154. This domain is also characterized as a “CheY-like receiver (REC) domain”, COG0784, in the Clusters of Orthologous Groups of proteins database and as an Interpro “CheY-like superfamily” domain, IPR011006, with both of these characterized domains extending from approximately amino acid 33 to approximately amino acid 161 of the Parachlorella SGI1 polypeptide of SEQ ID NO:3. The RR domain (sometimes referred to as a “receiver” or often, a “REC” domain) is found in bacterial two-component regulatory systems (like the bacterial chemotaxis two-component system that includes a polypeptide known as CheY), in which it receives a signal from a sensor partner. The RR domain of such systems is often found N-terminal to a DNA binding domain and typically includes a phosphoacceptor site that can be phosphorylated, where phosphorylation may be responsible for its activation or deactivation.

The Parachlorella SGI1 gene (T-2661 gene) also has a myb domain, positioned C-terminal to the RR domain. The myb domain is identified as pfam PF00249: “Myb-like DNA-binding domain”, extending from approximately amino acid position 204 to approximately amino acid position 254 of SEQ ID NO:3, and is also identified as conserved domain TIGRO1557 “myb-like DNA-binding domain, SHAQKYF class”, which extends from approximately amino acid position 202 to amino acid position 255 of SEQ ID NO:3 (Parachlorella SGI1 polypeptide). When SEQ ID NO:3 is queried against the Interpro protein sequence analysis and classification database, an Interpro Homeobox-like domain superfamily domain (IPR009057), extending from amino acid position 201 to amino acid position 259 of SEQ ID NO:3, and an Interpro Myb domain (IPR017930), extending from amino acid position 199 to amino acid position 258 of SEQ ID NO:3, are identified.

In addition, a nuclear localization signal could be identified in the Parachlorella SGI1 polypeptide positioned between the RR domain and the Myb domain, within a region of low amino acid conservation referred to herein as the linker between the conserved domains.

No conserved protein domains could be found in the region of the Parachlorella SGI1 polypeptide C-terminal to the myb domain. In contrast, the SGI1 architecture of an RR and a myb domains, where the myb domain is positioned C-terminal to the RR domain, can be found in many proteins coded for in Viridiplantae genomes. Bioinformatic analysis was used to identify likely orthologs of Parachlorella SGI1 in additional plant and algal species based on this conserved architecture.

To identify a class of SGI1 proteins in additional photosynthetic organisms, a Hidden Markov Model (HMM) was built for the ‘RR domain-myb domain’ architecture found in the Parachlorella SGI1 polypeptide. The amino acid sequence used to develop the HMM included the contiguous stretch of amino acids sequence that included both the RR domain and the myb domain, as well as the linker region between the two domains.

As a first step, the Parachlorella SGI1 polypeptide sequence (SEQ ID NO:3) was used to BLAST search the JGI Phytozome database v.12 that included the genomes of plants and algae. Three proprietary algal genomes (from Parachlorella, Tetraselmis, and Oocystis species) were also added to the database that was searched. The search was halted when it reached approximately 2,000 hits. These results were then analyzed by InterProScan (available from the EMBL-EBI [European Molecular Biolgy Laboratories-European Bioinformatics Institute, for example, at ebi.ac.uk]) to ensure that selected results had both the Interpro CheY-like superfamily domain (IPR011006) and the Interpro Homeobox-like or Myb domain (IPR009057 or IPR017930). Candidates that did not have both domains were eliminated. This step reduced the number of selected hits to between 900 and 1,000, with polypeptides having the two domain architecture (RR domain N-terminal to myb domain) clearly identifed in polpeptides of both algae and higher plants. The resulting sequences were used to assemble a phylogenetic tree based on sequence homology. The phylogenetic tree showed a clear grouping of SGI1-homologous polypeptides from algal species.

In a given species of algae, the number of SGI-like genes is low. Typically a single gene was identified in a given algal species, although in Tetraselmis three highly homologous genes were identified, which were in all likelihood alleles of the same gene the strain that was assessed to be triploid. The low number of SGI1 genes in a given algal species indicates that the identified SGI1 gene or genes in that algal species is in fact the functional SGI1 gene of that species, as the function of the single SGI1 polypeptide found in Parachlorella, which is haploid, and the three highly similar SGI1 polypeptides identified in Tetraselmis, which is thought to be triploid, were functionally validated (see Examples 5 and 7 and 16-18). (The Parachlorella and Tetraselmis genomes were sequenced in-house.)

To establish a criterion for likely SGI1 orthologs in other photosynthetic organisms then, a Hidden Markov Model (HMM) was developed based on the algal cluster of SGI1 polypeptide sequences. The HMM was developed based on the N-terminal portion of the SGI1 polypeptide that encompasses both the RR and myb domains, including the linker region between the two conserved domains. The sequence of the polypeptides C-terminal to the myb domain that did not include any recognizable conserved structure were excluded from the model-building. HMMER 3.1b2 was used to build the HMM using Multiple Sequence Alignments (MSAs) from proprietary sequences of Parachlorella, Oocystis, and Tetraselmis polypeptides as well as sequences of public databases of polypeptides of Chlamydomonas reinhardtii, Volvox carteri, Chromochloris zofingiensis, Coccomyxa subellipsoidea, Micromonas sp. RCC299, and Ostreococcus luminarinus. Multiple sequence alignments (MSAs) of the N-terminal portions of the proteins as described above were generated using the ETE3 toolkit and eggnog4l workflow. This program internally uses the programs Muscle, MAFFT, Clustal Omega, and M-coffee for alignment, trimAl for alignment trimming, and PhyML for phylogeny interference. All of these programs are publicly available. An HMM, unlike a single protein sequence used for homology comparison, for example, captures information from multiple protein sequences and is therefore able to distinguish highly conserved from highly divergent residues within a polypeptide sequence and take that into account when determining relatedness of sequences. When an HMM is used to score a sequence, highly conserved residues receive more weight that highly divergent residues, thereby providing superior sensitivity and acuracy than simpler PSAs.

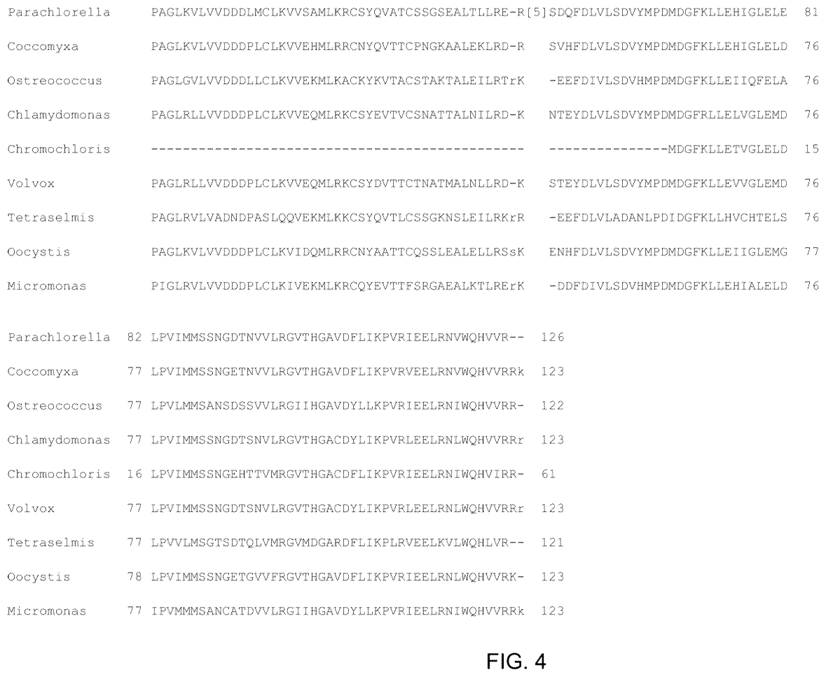

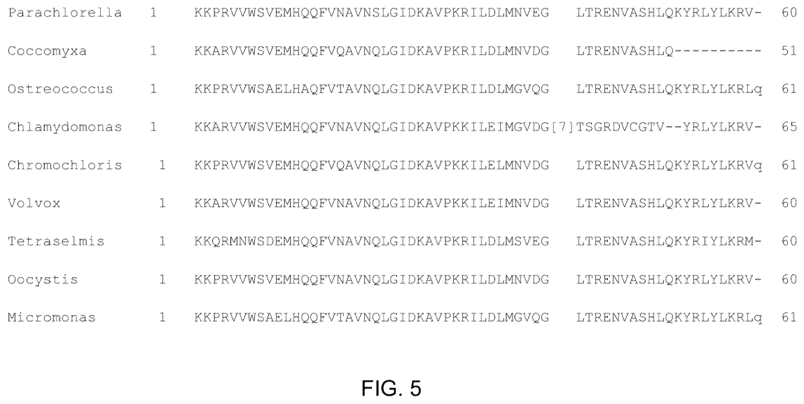

The SGI1 HMM was used to assign a score to the polypeptides identified in the BLAST search that also were verified as having the two conserved domains (RR and myb). The highest scores (with the exception of the very high-scoring Arabidopsis halleri homolog, SEQ ID NO:23) were found in algal species, with HMM scores for putative orthologs ranged from about 475 to less than 200 in the photosynthetic organism database. Algal homolog HMM scores in this sample generally ranged from about 400 to about 450. As shown in Table 3, the SGI1 homologs in Oocystis, Tetraselmis, Parachlorella and almost all additional algal homologs queried had scores of about 400 or greater. The unusually low score of the Chromochloris zofingiensis polypeptide is likely to be the result of flawed genome annotation; an alignment of RR domains of algal SGI1 polypeptides (FIG. 4) shows the sequence of the C. zofingiensis RR domain (SEQ ID NO:44) appears to be missing a continuous stretch of amino acids at the N-terminal region of this domain. Sequence homologies of the RR domains of the algal SGI1 homologs shown in FIG. 4 with the Parachlorella SGI1 RR domain (SEQ ID NO:40) ranged from about 55% to about 80% amino acid sequence identity. FIG. 5 provides an alignment of myb domain sequences of algal SGI1 polypeptides, with sequence homologies with the Parachlorella myb domain (SEQ ID NO: 58) ranging from about 85% to about 97% amino acid sequence identity among this set of algal homologs.

TABLE 3
SGI1 Orthologs in Algal Species

| | Polypeptide | RR | Myb | HMM | |
|---|---|---|---|---|---|
| Organism | Sequence | Domain | Domain | Score | E value |
| Parachlorella sp. | SEQ ID | SEQ ID | SEQ ID | 400.20 | 8.5e−118 |
| 1185 | NO: 3 | NO: 40 | NO: 58 | | |
| Coccomyxa | SEQ ID | SEQ ID | SEQ ID | 403.0 | 1.2e−118 |
| subellipsoidea | NO: 9 | NO: 41 | NO: 59 | | |
| Ostreococcus | SEQ ID | SEQ ID | SEQ ID | 425.8 | 1.4e−125 |
| lucimarinus | NO: 10 | NO: 42 | NO: 60 | | |
| Chlamydomonas | SEQ ID | SEQ ID | SEQ ID | 413.3 | 8.4e−122 |
| reinhardtii | NO: 11 | NO: 43 | NO: 61 | | |
| Chromochloris | SEQ ID | SEQ ID | SEQ ID | 292.6 | 6.1e−85 |
| zofingiensis | NO: 12 | NO: 44 | NO: 62 | | |
| Volvox carteri | SEQ ID | SEQ ID | SEQ ID | 441.4 | 2.3e−130 |
| | NO: 13 | NO: 45 | NO: 63 | | |
| Tetraselmis sp. 105 | SEQ ID | SEQ ID | SEQ ID | 403.6 | 7.9e−119 |
| (T-5172) | NO: 14 | NO: 46 | NO: 64 | | |
| Tetraselmis sp. 105 | SEQ ID | SEQ ID | SEQ ID | 403.0 | 1.2e−118 |
| (T-5185) | NO: 15 | NO: 46 | NO: 64 | | |
| Tetraselmis sp. 105 | SEQ ID | SEQ ID | SEQ ID | 402.9 | 1.3e−118 |
| (T-5230) | NO: 16 | NO: 46 | NO: 64 | | |
| Oocystis sp. | SEQ ID | SEQ ID | SEQ ID | 426.9 | 6e−126 |
| | NO: 17 | NO: 47 | NO: 65 | | |
| Micromonas sp. | SEQ ID | SEQ ID | SEQ ID | 418.4 | 2.4e−123 |
| RC299 | NO: 18 | NO: 48 | NO: 66 | | |
| Micromonas pusilla | SEQ ID | SEQ ID | SEQ ID | 405.9 | 1.6e−119 |
| | NO: 19 | NO: 49 | NO: 67 | | |

Plant species had homologs whose HMM scores ranged from 475.9 (Arabidopsis halleri) to below 200. Examples of plant homologs having HMM scores of 370 or greater are provided in Table 4. Growth effects on plants having a mutation in a gene encoding one of these homologs, the Arabidopsis gaditana “ARR2” gene having an HMM score of 371, is demonstrated in Example 19.

TABLE 4
SGI1 Homologs in Plant Species

| | Polypeptide | RR | Myb | | |
|---|---|---|---|---|---|
| Organism | Sequence | Domain | Domain | HMM Score | E value |
| Sphagnum fallax | SEQ ID NO: 20 | SEQ ID | SEQ ID | 397.3 | 6.8e−117 |
| | | NO: 50 | NO: 68 | | |
| Physcomitrella patens | SEQ ID NO: 21 | SEQ ID | SEQ ID | 372.3 | 2.8e−109 |
| | | NO: 51 | NO: 69 | | |
| Arabidopsis_thaliana | SEQ ID NO: 22 | SEQ ID | SEQ ID | 371.1 | 6.4e−109 |
| | | NO: 52 | NO: 70 | | |
| Arabidopsis halleri | SEQ ID NO: 23 | SEQ ID | SEQ ID | 475.9 | 6.9e−141 |
| | | NO: 53 | NO: 71 | | |
| Arabidopsis lyrata | SEQ ID NO: 24 | | SEQ ID | 395.5 | 2.4e−116 |
| | | | NO: 71 | | |
| Helianthus annus | SEQ ID NO: 25 | | | 391.2 | 4.9e−115 |
| Vitis vinifera | SEQ ID NO: 26 | SEQ ID | SEQ ID | 390.6 | 7.3e−115 |
| | | NO: 54 | NO: 72 | | |
| Amborella trichopoda | SEQ ID NO: 27 | | | 390.1 | 1e−114 |
| Ricinus communis | SEQ ID NO: 28 | | | 390.1 | 1.1e−114 |
| Solanum lycopersicum | SEQ ID NO: 29 | | | 388.4 | 3.4e−114 |
| Solanum tuberosum | SEQ ID NO: 30 | | | 387.2 | 7.9e−114 |
| Gossypium hirsutum | SEQ ID NO: 31 | | | 385.8 | 2.1e−113 |
| Theobroma cacao | SEQ ID NO: 32 | | | 383.0 | 1.6e−112 |
| Phaseolus vulgaris | SEQ ID NO: 33 | | | 381.6 | 4.2e−112 |
| Glycine max | SEQ ID NO: 34 | SEQ ID | SEQ ID | 381.4 | 4.6e−112 |
| | | NO: 55 | NO: 73 | | |
| Chenopodium quinoa | SEQ ID NO: 35 | | | 373.7 | 1.1e−109 |
| Malus domestica | SEQ ID NO: 36 | | | 372.6 | 2.4e−109 |
| Zea mays | SEQ ID NO: 37 | SEQ ID | SEQ ID | 371.5 | 4.9e−109 |
| | | NO: 56 | NO: 74 | | |
| Brassica rapa | SEQ ID NO: 38 | | | 370.5 | 1e−108 |
| Oryza sativa | SEQ ID NO: 39 | SEQ ID | SEQ ID | 369.6 | 1.9e−108 |
| | | NO: 57 | NO: 75 | | |

In all of the algal and plant homologs listed in Tables 2 and 3, the RR and myb domains occur within the first (N-terminal-most) amino acids of the polypeptide sequences as annotated in the JGI genomes, and no conserved domains are identified carboxy-terminal to the myb domain in these SGI1 homologs.

### Example 7

### Chlorophyll Content, Antenna Size, and Photophysiology of SGI1 Mutants Ne-7843 and Ne-13380

Chlorophyll content of the high productivity mutants was determined by extracting cells with methanol and analyzing the supernatant by spectrophotometry. Briefly, 500 μl aliquots of culture were pipetted into 2.0 ml twist top tubes and pelleted using a table top microcentrifuge at 15,000 rpm for 10 minutes. The supernatants were aspirated from the pellets, and each pellet was resuspended in 1.5 ml 99.8% methanol (previously neutralized with magnesium carbonate). 0.2 ml of glass beads (0.1 mm diameter) were added to each vial and bead beat for 3 min. 1.0 ml of supernatant was transferred to new 1.7 ml flip-top tubes and were centrifuged in a table top microcentrifuge at 15,000 rpm for 10 minutes. The resulting pellets were white indicating that a complete extraction had been performed. 0.8 ml of each supernatant was pipetted into an optical glass cuvette and absorption wavelengths were read immediately at 720 nm, 665 nm and 652 nm wavelengths. Spectrophotometric measurements were carried out in dual-beam mode using a 99.8% methanol blank. The following equations were used to calculate the concentration of chlorophyll: Chlorophyll a [g m⁻³]=16.72(A665-A720)+9.16 (A652-A720) and Chlorophyll b [g m⁻³]=34.09(A652-A720)−15.28(A665-A720). The amount of chlorophylls a and be were standardized on a per cell and per TOC basis. Table 5 shows that while the amount of total chlorophyll per cell varied somewhat among the SGI1 mutants, it was universally decreased with respect to wild type cells by an amount ranging from about 30% to about 65%, consistent with the observed reduction in antenna size. On a per TOC basis, the reduction in total chlorophyll in SGI1 mutants with respect to wild type cells ranged from about 30% to about 50%.

TABLE 5
Chlorophyll Content of SGI1 Mutants and Wild Type Algae

| | Chl a/ | Chl a, | Chl b/ | Chl b, | Total Chl/ | Total Chl/ |
|---|---|---|---|---|---|---|
| | cell, | % | cell, | % | TOC | TOC % |
| Strain | pg | change | pg | Change | (%) | Change |
| WT-1185 | 0.284 | 0% | 0.097 | 0% | 6.9% | 0% |
| NE-7843 | 0.306 | 8% | 0.058 | −40% | 4.7% | −32% |
| NE-13380 | 0.287 | 1% | 0.063 | −35% | 4.4% | −36% |
| GE-16491 | 0.250 | −12% | 0.045 | −54% | 3.7% | −46% |
| GE-16492 | 0.211 | −26% | 0.035 | −64% | 3.4% | −50% |
| GE-16493 | 0.246 | −14% | 0.043 | −55% | 3.6% | −47% |

In addition to chlorophyll content, SGI1 knockout strains NE-7843, NE-13380, GE-16391, GE-16392, and GE-16393 were analyzed for PSII antenna size, PSI antenna size, 1/τ′_{Qa} (the light saturated rate of electron transport on the acceptor side of photosystem II at light saturation, a measure of the efficiency of linear photosynthetic electron transport) as well as a Pmax for carbon fixation. Cells of the wild type and SGI1 mutant strains were cultured in the constant light semicontinuous culture assay (CL-SCPA) described in Example 3. Data on Parachlorella wild type and SGI1 classical mutants (NE-7843 and NE-13380) were obtained in separate experiments from the that included the three targeted SGI1 knockouts (GE-16491, GE-16492, GE-16493). Analysis of various photosynthetic parameters was performed using the Fluorescence Induction and Relaxation (FIRe) technique developed to measure a comprehensive series of photosynthetic and physiological characteristics of photosynthetic organisms (Gorbunov and Falkowski (2005) “Fluorescence Induction and Relaxation (FIRe) Technique and Instrumentation for Monitoring Photosynthetic Processes and Primary Production in Aquatic Ecosystems” in: Photosynthesis: Fundamental Aspects to Global Perspectives, Proc. 13th International Congress of Photosynthesis, Montreal, Aug. 29-Sep. 3, 2004. (Eds: A. van der Est and D. Bruce), Allen Press, V.2, pp. 1029-1031). The FIRe technique relies on measurement and analysis of chlorophyll “variable fluorescence” profiles (reviewed by Falkowski et. al., 2004 “Development and Application of Variable Chlorophyll Fluorescence Techniques in Marine Ecosystems” in: Chlorophyll a Fluorescence: A Signature of Photosynthesis (C. Papageorgiou and Govingjee, eds), Springer, pp. 757-778) which depend on the relationship between chlorophyll fluorescence and the efficiency of photosynthetic processes. This technique provides a set of parameters that characterize photosynthetic light-harvesting processes, the photochemistry in Photosystem II (PSII), and photosynthetic electron transport down to carbon fixation. The measurements performed herein used a mini-FIRe device produced by Maxim Gorbunov aof Rutgers University, East Brunswick, NJ. A commercially available FIRe device is available from Sea-Bird Scientific (Halifax, Canada, satlantic.com and planet-ocean.co.uk). Further information regarding the use of the FIRe device is available in company manuals. All measurements were taken using constant light (2000 μmol photons·m⁻²·sec⁻¹) semicontinuous cultures (CL-SCPA) cultures (see Example 3). To obtain F_{V}/F_{M} and σ_{PSII} measurements of Fluorescence Induction and Relaxation (FIRe) kinetics were performed in the dark. The values for Fv/Fm and σ_{PSII} presented in Table 7 were calculated as an average of 6 measurements (3 measurements of each of the 2 biological replicates), errors for these parameters did not exceed 5%.

Measurements of PSI cross-section were performed using a modified JTS-10 spectrometer with a filter set to measure the electrochromic shift (ECS) at 520 nm equipped with a custom-built single turnover flasher (STF). The peak power density in the sample chamber was high enough to ensure full closure of reaction centers within approximately 10 μs. The resulting excitation rate was ˜1 to 3 hits per reaction center per 10 μs (depending on the functional absorption cross section of the photosystem). The STF generated short ultra-bright pulses of blue light (455 nm, with 30 nm half bandwidth), and the pulse timing was controlled by the trigger from the JTS-10 Spectrometer. The pulse duration was controlled by the STF Pulse Control Box and was adjustable in the range from 1 μs to 50 μs using the potentiometer on the front panel. To measure the PSI cross-section, we diluted cultures to an OD of about 0.2 at the chlorophyll maximum (˜440 nm) based on measurement of absorption spectra of cell suspension using a Perkin Elmer Lambda 650 spectrophotometer equipped with an integrating sphere. The ECS was measured using 10 μs flashes with intensities ranging from 4000 to 120,000 μmol photons m⁻² s⁻¹ in the presence of DCMU and hydroxylamine. The experimental curve was fitted with a simple exponential function

ECS=ECS_{M}×(1−e^{lt×σ}^{_{PSI}})

where ECS M is the maximal ECS signal; It is photon density in photons/m²; and σ_{PSI} is functional cross-section of PSI. Obtained values for functional cross-section of PSI for the wild type of Parachlorella (WT-1185) was (4.0±0.5)×10⁻¹⁸ m². These values are close to those obtained for th functional cross-section of PSII grown under the same conditions (σ_{PSII}=(4.3±0.1)×10⁻¹⁸ m²). Errors for these parameters were estimated not to exceed 20%.

Carbon fixation rates (C¹⁴ Pₘₐₓ) were measured using cultures normalized to 5 μg chl ml⁻¹ in media containing 0.5 g l⁻¹ (5.95 mM) sodium bicarbonate. 20.4 μCi ml⁻¹ C¹⁴ labeled sodium bicarbonate was added to each culture and exposed to 2500 μE for a duration of 10 minutes. Samples were immediately acidified with 2N HCl and allowed to off-gas overnight. The following day samples were measured using a Beckman LS6500 scintillation counter and quantified.

τ′_{Qa} (the time of electron transport on the acceptor side of PSII measured under saturating light conditions−effectively determined by the slowest step of linear photosynthetic electron transport) was measured from FIRe light curves and dark induced relaxation kinetics (DIRK) profiles. Volumetric PSII concentration relative to wild type was estimated as (Fv/σ⁵³⁰_{PSII}). Errors for these parameters were estimated not to exceed 15%. Optical absorption cross section (averaged over emission spectrum of a light source) was estimated using the following equation:

$${a}_{chl / TOC} = \frac{1}{[ Chl / TOC ]} {\int }_{400}^{700} ln ( 10 ) \times \frac{OD ( λ )}{Δ l} \times \frac{I ( λ )}{{\int }_{400}^{700} I ( λ ) d λ} d λ$$

where [Chl/TOC] is the chlorophyll/TOC of the sample, OD(λ) is the measured optical density of the sample at wavelength λ, Δl is the measuring beam pathlength in the cuvette (1 cm), I(λ) is the intensity of the light source used to grow algae at wavelength λ.

TABLE 6
Fluorescent and Photosynthetic Parameters Measured with the FIRe Technique

| FIRe, JTS-10 | |
|---|---|
| Retrieved | |
| Parameters | Description |
| Fv/Fm | Maximum quantum yield of photochemistry in PSII, measured in a dark- |
| | adapted state (dimensionless). This parameter characterizes the efficiency |
| | of primary photosynthetic reactions. |
| OPSII | Functional absorption cross section of PSII (Å²) in a dark-adapted state. |
| | The parameter is the product of the optical absorption cross section of PSII |
| | (i.e., the physical size of the PSII unit) and the quantum yield of |
| | photochemistry in PSII. Could be measured using different excitation |
| | wavelengths |
| OPSI | Functional absorption cross section of PSI (Å²) in a dark-adapted state. The |
| | parameter is the product of the optical absorption cross section of PSII (i.e., |
| | the physical size of the PSI unit) and the quantum yield of photochemistry |
| | in PSI. |
| 1/t'Qa | Light saturated rate of electron transport on the acceptor side of |
| | photosystem II. This parameter indicates efficiency of linear photosynthetic |
| | electron transport |

Table 7 shows that all of the SGI1 mutants demonstrated increased Fv/Fm (by about 10-14%) while exhibiting reduced antenna cross-sectional size as compared to the wild type strain from which they were derived. The SGI1 mutants also had reduced antenna size of PSII and PSI (down 40-50% with respect to wild type), high rates of electron transport on the acceptor side of PSII_{(1/τ′}_{_{Qa}}₎ under saturating light (increased between about 35% and about 130%, and by at least approximately 100% with respect to wild type in the engineered mutants) and high rates of carbon uptake (Pₘₐₓ) (up at least 30-40% with respect to wild type), while, as determined by Multiple Reaction Monitoring protein determination, the number of photosystems on a per TOC basis was maintained (see Example 7). Strains were also characterized by higher chlorophyll a to chlorophyll b ratio compared to the wild type (increased by approximately 70%), which is indicative of loss of peripheral light harvesting complexes and is consistent with the observed reduced size of the functional cross-section of PSII. (Table 7). The recapitulated SGI1 KO mutants (GE-16491, GE-16492, and GE-16493, engineered using CRISPR/Cas9) showed a very similar photophysiological phenotype to the original mutants, but with even greater reductions in the antenna of PSII (up to 55%), faster rates of electron transport on the acceptor side of PSII under saturating light (increased by up to 130%), and higher rates of carbon uptake (up to 80% higher in GE-16491) (Table 7).

TABLE 7
Photophysiology of Parachlorella SGI Mutants

| | | | | | | ¹⁴C | Cell |
|---|---|---|---|---|---|---|---|
| | FIRe/JTS-10 | | | | | Pₘₐₓ | Size |
| | | σ_{PSII} | σ_{PSII} | σ_{PSI} | | (nmol ¹⁴C/ | Cell |
| | | (Å², at | (Å², at | (Å², at | 1/τ'_{Qa} | μg TOC/ | size, |
| Strain | F_{V}/F_{M} | 450 nm) | 530 nm) | 440 nm) | (s⁻¹) | hour) | um³ |
| WT-1185 | 0.598 | 428 | 153 | 380 | 94 | 10.1 | 40 |
| NE-7843 | 0.667 | 277 | 89 | — | 194 | 14.8 | 63 |
| NE-13380 | 0.677 | 261 | 84 | 240 | 197 | 17.3 | 57 |
| GE-16491 | 0.678 | 253 | 75 | — | 211 | 19.6 | 83 |
| GE-16492 | 0.688 | 235 | 69 | — | 225 | 15.4 | 85 |
| GE-16493 | 0.687 | 243 | 71 | — | 250 | 16.2 | 76 |

### Example 8

### Photophysiological Screening for High Productivity Algal Mutants

Based on the discovery of high productivity mutants NE-7843 and NE-13380 using a screen for reduced antenna strains and productivity assays, and the subsequent discovery that such high productivity mutants had distinctive photophysiological characteristics as set forth in Tables 5 and 6, including higher maxium photosynthetic yield (F_{V}/F_{M}), reduced PSII antenna size (σ_{PSII}), and increased PSII turnover rate (1/τ′_{Qa}), a screen for high productivity algal mutants was devised, referred to herein as the “FACS-FIRE” screen. The screen included a selection for low chlorophyll mutants from a progenitor algal population using FACS, and screening of individual lines from the population selected by FACS for low chlorophyll fluorescence using the FIRe device described in Example 6 to determine PSII antenna size (σ_{PSII}) at one or more wavelengths, F_{V}/F_{M}, and the rate of electron transport at the acceptor side of PSII (1/τ′_{Qa}). Lines having reduced PSII antenna size, increased F_{V}/F_{M} and increased 1/τ′_{Qa} were expanded, re-tested, and assayed for carbon uptake rate (Pm (¹⁴C)) and productivity in culture.

In pilot experiments, wild type Parachlorella cells (strain WT-1185) were mutagenized by UV as described in Example 1. Mutagenesis can also be effected by chemical mutagens, random insertional mutagenesis (see, for example, US 2014/0220638, incorporated herein by reference) or even targeted mutagenesis, such as by CRISPR/Cas9 (see, for example US 2016/0304896, incorporated herein by reference).

As described in Example 2, mutagenized algal cells were allowed to recover in low light and then subjected to fluorescence activated cell sorting (FACS), where low chlorophyll fluorescence cells having the lowest approximately 0.5 to 2% of chlorophyll fluorescence compared to the total population of cells were selected. (Sticter, less strict, broader, or narrower ranges of chlorophyll fluorescence can be imposed on the FACS selection at the user's discretion.) The FACS sorted cells were plated, and after colonies grew up they were individually picked and inocculated into culture medium (lacking a reduced carbon source) in 96 well plates and the cultures were allowed to grow photoautotrophically in the light (500 μE m⁻² s⁻¹. Small-scale (approximately 100-200 μl) cultures were initiated in ten 96 well plates, resulting in approximately 960 individual cultures of low chlorophyll-selected lines.

The second phase of the screen for high productivity mutants used the FIRe device with an attached fiber optic cable that transferred light pulses for probing cell fluorescence and transmitted collected fluorescence to the instrument detector. The fiber optic cable was held over each individual well to probe and measure fluorescence from isolated cell lines, which was recorded and used to calculate F_{V}/F_{M}, PSII antenna size (σ_{PSII}), and PSII turnover rate (rate of electron transport at the acceptor side of PSII, 1/τ′_{Qa}) by the FIRe device. Lines exhibiting F_{V}/F_{M} increased by at least 10%, PSII antenna size reduced by at least 30%, and PSII turnover rate increased by at least 50% were selected for further study. Cutoff values for such parameters can be set to different values at the user's discretion, for example, F_{V}/F_{M} increased by at least 5%, PSII antenna size reduced by at least 20%, and PSII turnover rate increased by at least 20% or stricter criteria can be used for selecting strains of interest.

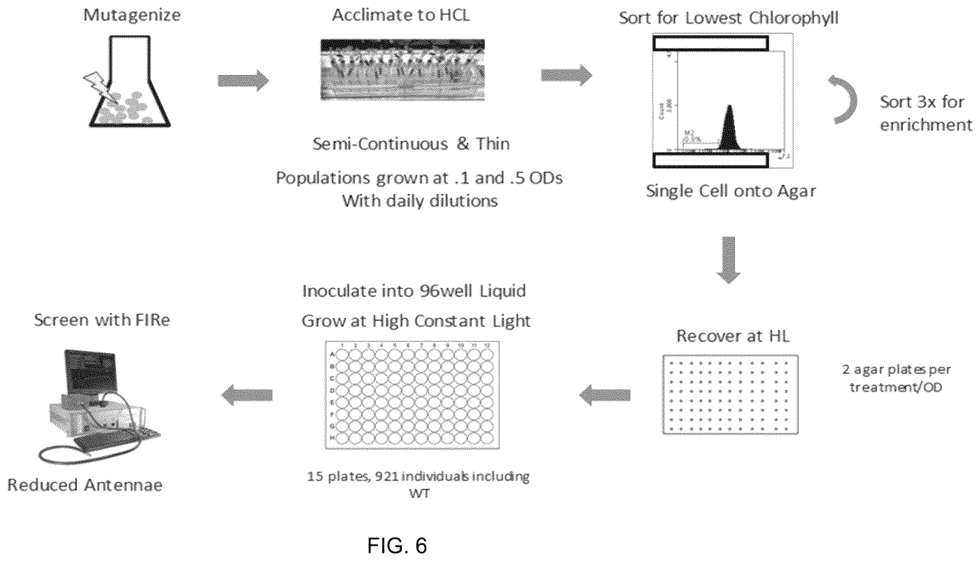

A schematic of the screeing process is shown in FIG. 6. The screen, involving FACS-based selection of low chlorophyll lines and FIRe-based screening of small scale culture can be further automated, for example, by the FACS device directing low chlorophyll cells directly into wells of multiwell plates and/or by an automated mechanical arm to successively position the FIRe fiber optic cable or an emitting LED with fluorescence detector system—or another system capable of providing microsecond high intensity light flashes and collecting fluorescence signals over individual wells of multiwell plates.

Algal lines demonstrating low chlorophyll fluorescence and reduced antenna size, increased Fv/Fm, and increased PSII turnover rate (1/τ′_{Qa}) can be further assessed as provided in Example 6, above, for example, for low chlorophyll b content and/or increased carbon uptake and for higher productivity in culture, for example, in semicontinuous culture (e.g., Example 3).

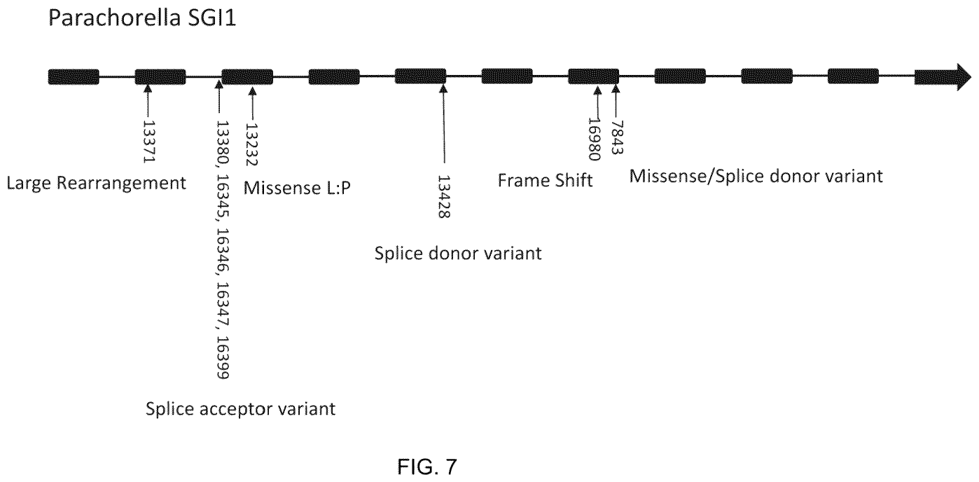

Several mutant strains were identified using the FACS-FIRE screening methods, among them NE-16980, whose genome was sequenced. NE-16980 was found to have a frameshift mutation in the SGI1 (T-2661) gene (FIG. 7).

### Example 9

### Microproximate Analysis of SGI1 Mutants NE-7843 and NE-13380

To determine the overall biomass composition of the SGI1 attenuation mutants, quantitative analysis of samples from cultures grown in semicontinuous mode with 40% daily dilution was performed to determine the lipid, protein, and carbohydrate content of the cells in semi-continuous culture. After the cultures reached steady state, aliquots of the culture removed for daily dilution was used for analysis of lipid, protein, and carbohydrate. Total organic carbon (TOC) of the algal culture samples was determined by diluting 2 mL of cell culture to a total volume of 20 mL with DI water. Three injections per measurement were injected into a Shimadzu TOC-Vcsj Analyzer for determination of Total Carbon (TC) and Total Inorganic Carbon (TIC). The combustion furnace was set to 720° C., and TOC was determined by subtracting TIC from TC. The 4 point calibration range was from 2 ppm to 200 ppm corresponding to 20-2000 ppm for non-diluted cultures with a correlation coefficient of r²>0.999.

To determine lipid content, FAME analysis was performed on 2 mL samples that were dried using a GeneVac HT-4X. To the dried pellets the following was added: 500 μL of 500 mM KOH in methanol, 200 μL of tetrahydrofuran containing 0.05% butylated hydroxyl toluene, 40 μL of a 2 mg/ml C11:0 free fatty acid/C13:0 triglyceride/C23:0 fatty acid methyl ester internal standard mix and 500 μL of glass beads (425-600 μm diameter). The vials were capped with open top PTFE septa-lined caps and placed in an SPEX GenoGrinder at 1.65 krpm for 7.5 minutes. The samples were then heated at 80° C. for five minutes and allowed to cool. For derivatization, 500 μL of 10% boron trifluoride in methanol was added to the samples prior to heating at 80° C. for 30 minutes. The tubes were allowed to cool prior to adding 2 mL of heptane and 500 μL of 5 M NaCl. The samples were then vortexed for five minutes at 2 krpm and finally centrifuged for three minutes at 1 krpm. The heptane layer was sampled using a Gerstel MPS Autosampler. Quantitation used the 80 μg of C23:0 FAME internal standard.

To determine protein content, isolated biomass samples were hydrolyzed and the amino acids were derivatized to propoxycarbonyl propyl esters (AAPE's), analyzed via GC/MS, and quantitated against an internal standard, as detailed below. Aliquots (0.5 ml) of wild type Parachlorella (WT-1185) and SGI1 knockout strain GE-13380 from the semicontinuous cultures were centrifuged and the pellets were washed twice with phosphate buffered saline (PBS). The cells were finally resuspended to a final volume of 0.5 ml (the starting volume) and transferred to a 4 ml glass vial. To the culture sample, 800 μl of 6M HCl with TGA was added (400 μl of thioglycolic acid (TGA) was added to 19.6 ml of 6M HCl just before use). Ten μl of beta mercaptoethanol were then added to the vial, followed by 200 μl of 20 mM norvaline, used as an internal standard. Each vial was blanketed with N₂ for 10 seconds, after which the vials were vortexed for 1 min at 2500 rpm to homogenize the samples. The vials were then placed in a 110° C. oven for 22 hours. At the end of the hydrolysis incubation, the vials were vortexed for 10 min at 2500 rpm, and then centrifuged up to 1000 rpm after which the centrifuge was stopped. A 50 aliquot was removed from each vial and dried by placing in an acid safe EZ-2 Genevac which was run on the HCl method for at least 3 hours prior to derivatization.

For derivativization, 250 μl of milli-Q H₂O was added to the dried acid hydrolysates, followed by 10 μl of antioxidant mix and then 120 μl of 0.5M NaOH. The antioxidant mix was made by adding 0.25 ml of n-propanol, 50 μl of thiodiglycol, and a few granules of phenol to 2.20 ml of Milli-Q H₂O, and vortexing. 80 μl of the catalyst, a 4:1 mix of pyridine and n-propanol was then added, and the vial was capped and vortexed at 2500 rpm for 1 min. 50 μl of propyl chloroformate was then added, and the vial was capped and vortexed at 2500 rpm for 1 min. After a 1 min incubation, the vial was again vortexed at 2500 rpm for 1 min. 500 μl of a 4:1 mixture of isooctane and chloroform was then added to the vial which was again capped and vortexed at 2500 rpm for 1 min. The rack of sample vials was then covered with another sample rack and shaken 20 times to ensure emulsion of the samples. The samples were then centrifuged until the centrifuge reached 1000 rpm and then the centrifuge was stopped. 200 μl of the organic layer was removed into a new GC vial with a glass insert and analyzed by GC/MS.

The samples were analyzed by GC/MS using a ZB-AAA 10×0.25 mm ID Amino Acid Analysis GC column and quantitated using the internal norvaline standard. The needle Wash 1 solvent was acetone and the needle Wash 2 solvent was isooctane/chloroform (80/20) with a program of 110° C., hold 0 min, 30° C./min to 320° C., hold 0.5 min, using a 4 μl injection at 15:1 split, 250° C. at 1.1 ml/min with a 300° C. transfer line.

The GC-MS data was multiplied by 0.0005 L to obtain μmol values, and multiplied by the molecular weight of the amino acid. The value was divided by 5 to correct for the volume to obtain μg/ml of each amino acid. Asparagine is converted to aspartic acid during acid hydrolysis, thus asparagine plus aspartic acid are determined as aspartic acid in these methods. Tryptophan is not measured by these methods but does not make up a significant fraction of the amino acids in Parachlorella proteins.

For total carbohydrate quantitation, biomass was hydrolyzed for one hour in 6N hydrochloric acid to convert polysaccharides to monosaccharides. The resulting monosaccharides were converted to trimethylsilyl ethers using MSTFA N-methyl-N-trinethylsilyltrifluoroacetamide with 1% trimethylchlorosilane, and the ethers were resolved and quantitated using GC-MS analysis.

For acid hydrolysis of culture samples, 500 μl of Milli-Q H₂O was added to 500 μl culture samples in 4 ml vials, or, where the culture sample was more concentrated (higher TOC), 800 μl of Milli-Q H₂O was added to 200 μl of culture sample. 20 μl of 2.5 mg/ml ribitol and U-¹³C-glucose as an internal standard was added to the 1 ml diluted culture samples in 4 ml vials. 1 ml of concentrated HCl was then added to each of the vials, the vials were capped and placed in a 105° C. dry bath for 1 hour. The samples were then allowed to cool to room temperature, and 100 μl was transferred to a glass insert inside a 1.5 ml microcentrifuge tube.

For derivatization, the microfuge tubes that included glass inserts containing the samples were place in an acid safe EZ-2 Genevac which was run on the HCl method for at least 3 hours. After drying, 100 μl of the derivatization reagent, which consisted of 800 μl of dry pyridine added to 1 ml of freshly opened MFSTA-1% TMCS, was added to each sample. The samples were incubated for 1 hour at 40° C. while mixing at 1000 rpm in an Eppendorf Thermomixer. Following incubation, the samples were directly analyzed by GC/MS.

The samples were analyzed by GC/MS using a DB5-MS 30 m×250 μm×25 μm GC column and quantitated using the internal U-¹³C-glucose standard. The needle wash solvent was pyridine with a program of 1 min equilibration, 170° C. for 8 min, 10° C./min to 210° C. for 0 min, then 50° C./min to 325° C. for 2 min (total run time 16.3 min).

FIG. 8 shows the results of this analysis on the wild type Parachlorella strain as well as the SGI1 gene attenuation knockout mutants GE-13380. Unexpectedly, SGI1 knockout mutant NE-13380 was observed to have had an increased percentage of its total organic carbon as protein with respect to wild type, demonstrating an approximately 20% increase in protein on a per TOC basis with respect to the wild type strain (WT-1185). The SGI1 mutant, which as demonstrated in Example 3 had increased total organic carbon accumulation as compared to wild type in the semi-continuous assay, demonstrated correspondingly reduced lipid and carbohydrate. The amino acid composition of the protein of SGI1 mutants was found to be highly similar to that of wild type cells. The SGI1 mutant strains therefore provide a genetic means of increasing the protein content of algae and algal biomass. Depending on culture conditions, an increase of approximately 40% to approximately 60% in areal protein productivity can be anticipated when compounding the increased percentage of protein with the increased biomass productivity demonstrated by the SGI1 mutants.

### Example 10

### Transcriptomics and Proteomics of SGI1 Mutants NE-7843 and NE-13380

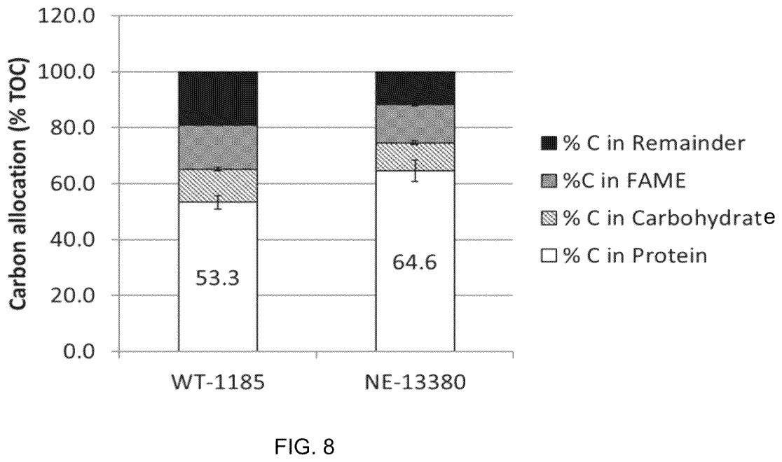

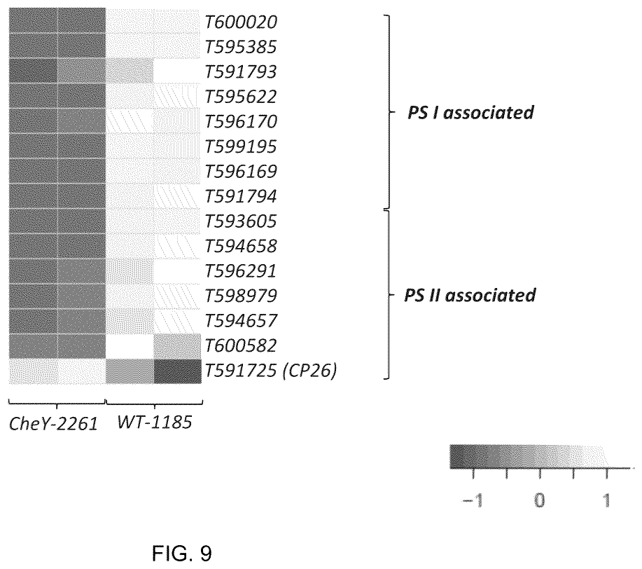

Transcriptomic analysis was performed on mRNA extracts of the wild type and SGI1 mutant strains NE-7843 and NE-13380 cultured in the constant light semicontinuous culture assay (CL-SCPA or “CL2000” assay) described in Example 3. Transcriptomic analysis of SGI1 mutants showed a large number of differentially regulated genes with respect to wild type. As the SGI1mutants demonstrated reduced chlorophyll and reduced photosynthetic antenna, it was of interest to see whether the light harvesting chlorophyll-binding protein (LHC) genes had lower transcript abundance in the mutants with respect to the wild type progenitor strain. Fifteen LHC genes were identified in the Parachlorella genome, and strikingly, with the exception of the gene encoding CP26, an LHC polypeptide associated with the PSII reaction center, all of the identified Parachlorella LHC genes were found to be downregulated in the SGI1 mutants with respect to wild type expression levels (FIG. 9). On average, the LHCs mRNAs showed about a 1.7 fold reduction in abundance in SGI1 relative to WT-1185. Another striking observation from these transcriptomics data was that Rubisco activase was found to have approximately 6 fold higher transcript abundance in the SGI1 mutant.

Global proteomic analysis was also performed for the WT-1185 and SGI1 mutant NE-7843 cultured in the constant light semicontinuous culture assay (CL-SCPA or “CL2000” assay) described in Example 3. Mass spectrometry analysis (Michigan State University Proteomics Core Facility, East Lansing, MI) was performed to determine global protein abundance. Consistent with the transcriptomics dataset, the SGI1 mutant showed reduced LHC protein abundance and elevated Rubisco activase abundance with respect to wild type cells.

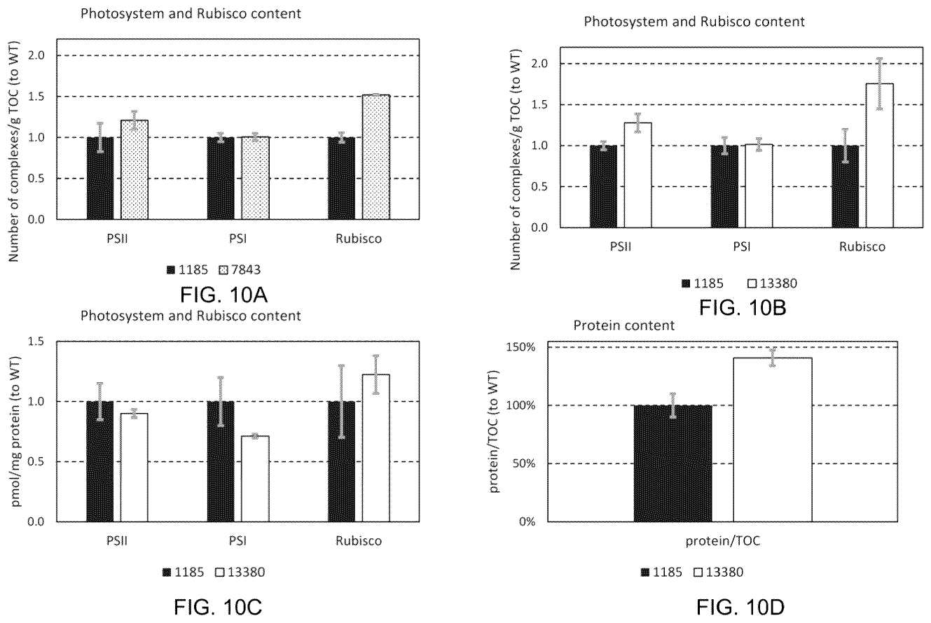

Targeted proteomic analysis was also performed for wild type and SGI1 mutant strains (NE-7843 and NE-13380) cultured in the constant light semicontinuous culture assay (CL-SCPA or “CL2000” assay) described in Example 3. Proteomic analysis was performed using Multiple Reaction Monitoring (MRM), a mass spectrometry approach than enables targeted quantification of proteins of interest with high specificity and sensitivity using internal standards for each protein of interest (see, for example, Qu et al. (2006) Anal Chem. 78:4543-4552; Domon et al. (2006) Mol. Cell. Proteomics 5:1921-1926; Wolf-Yadlin et al (2007) Proc. Natl. Acad. Sci. USA 104:5860-5865; Stahl-Zeng et al. (2007) Mol. Cell. Proteomics 6:1809-1817). High quality peptides from PsaD (a PS I subunit) PsbB (a PS II subunit) and RbcL (the Rubisco large subunit), were used to quantitate PSI, PSII and Rubisco abundance in wild type Parachlorella (WT-1185) and SGI1 mutants (NE-7843 and NE-13380). The results of the MRM proteomics analysis (JadeBio, La Jolla, CA) indicated that the SGI1 mutants are characterized by a slightly elevated PSII/TOC content and PSII:PSI ratio (FIGS. 10A and 10B). Higher Rubisco/TOC content was also observed in the SGI1 mutants compared to the wild type (FIGS. 10A and 10B), which, consistent with the microproximate analysis (Example 6), had higher protein content per TOC. However, quantitative Westerns comparing the levels for PsbD (a PS II polypeptide), the Rubisco large subunit, and Rubisco activase showed that, when normalized to total protein, PS II and Rubisco levels were comparable in wild type and SGI1 mutants, as the SGI1 mutant has a higher protein/TOC content overall (FIGS. 10C and 10D).

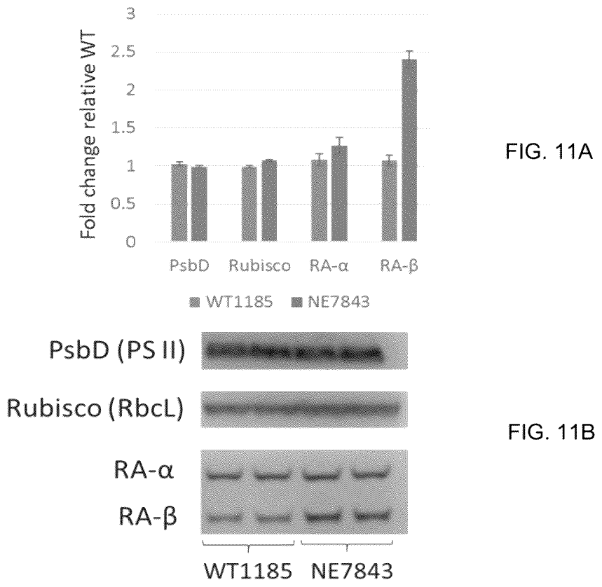

An increase in the abundance of the Rubisco activase (RA) protein was observed in both transcriptomics and proteomics analysis (FIGS. 11A and 11B). These observations were confirmed through quantitative Western analysis. The two Rubisco activase isozymes (alpha and beta) mainly differ in their N-termini, with RA-α having an extended N-terminus with respect to RA-β. FIG. 11B shows a Western blot in which the alpha isozyme of the Rubisco activase protein (RA-α) was found to be increased slightly and the beta isozyme of the Rubisco activase protein (RA-β) was found to be significantly increased in abundance to approximately 2.5 fold the wild type protein level, consistent with the increased abundance of the RA transcript in the mutant as determined by transcriptomic analysis.

### Example 11

### Productivity of SGI1 Mutants in Semicontinuous Culture Simulating Natural Irradiance Conditions

To assess productivity of SGI1 mutants in a semicontinuous system under a light regime that mimicked natural daylight conditions on a spring day in Southern California, scale-up cultures were used to inoculate 225 cm² rectangular tissue culture flasks, each of which contained a final total volume of 550 ml of culture after inoculation. A typical inoculum volume was approximately 200 ml of scale-up culture that was added to approximately 350 ml of PM119 culture medium. Cultures were diluted daily at mid-day, when the light intensity was at its peak, by removing 65% of the volume and replacing it with the same volume of the assay medium plus an additional 10 ml of deionized water to make up for evaporation (included in the make-up medium). Semi-continuous assays were typically run for 10-14 days. Daily lipid and biomass productivities were only calculated for cultures that had reached steady state (where the increase in growth was equal to the dilution factor for the assay).

Three cultures were initiated per strain. The flasks included stir bars and had stoppers having inserted tubing connected with syringe filters for delivering CO₂ enriched air (1% CO₂, flow rate, 300 ml per min) that was bubbled through the cultures. The flasks were set in a water bath programmed to maintain a constant temperature of 25° C. on stir plates set to 575 rpm during the assay period. Culture flasks were masked with an opaque white plastic to provide a 31.5 cm² rectangular opening for irradiance to reach the culture. The flasks were aligned with the width (narrowest dimension) against an LED light bank that was programmed with a light/dark cycle and light profile that increased until “solar noon” and then declined to the end of the light period. The light profile was designed to mimic a spring day in Southern California: 14 h light:10 h dark, with the light peaking at approximately 2000 μE. The flasks included stir bars and had stoppers with inserted tubing connected with syringe filters for delivering CO₂ enriched air (1% CO₂, flow rate, 300 ml per min). The flasks were set in a water bath programmed to maintain a constant temperature of 25° C. on stir plates set to 575 rpm during the assay period.

Daily biomass (TOC) productivities were calculated from cultures that had reached steady state. Volumetric TOC productivities in (mg/L/day) were calculated by multiplying the volumetric TOC amounts by the dilution rate. Areal productivities (g/m²/day) were calculated by dividing the total productivity of the culture by the size of the aperture through which irradiance was permitted:

$$\frac{( volumetric productivity ) mg}{L * day} * \frac{0.55 L}{0.00315 {m}^{2}} * \frac{g}{1000 mg} = \frac{g}{{m}^{2} * day}$$

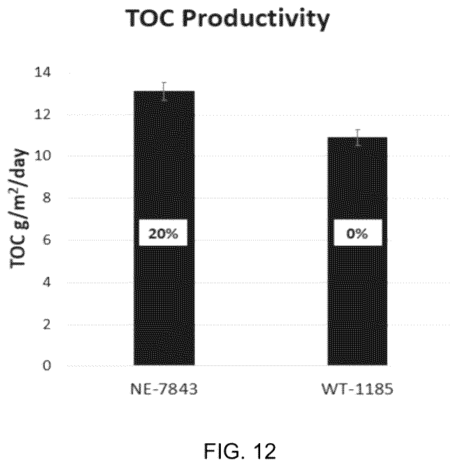

FIG. 12 shows the average daily TOC productivity for triplicate cultures of the NE-7843 SGI1 mutant and wild type strain WT-1185 in the semicontinuous diel assay, showing that under these conditions the mutant exhibited a 20% increase in productivity with respect to wild type.

### Example 12

### Productivity of Batch Cultures Under Nutrient Replete Conditions

Productivity of SGI1 mutant NE-13380 was also assessed over one week while cultured in nitrogen replete batch mode. Cultures of NE-13380 and wild type WT-1185 were inoculated to an initial OD730 of 0.5 from seed (scale-up) cultures that were grown in nitrogen replete culture medium.

After inoculation, SGI1 knockout strain NE-13380 and wild type strain WT-1185 were grown in triplicate cultures in a batch assay in 75 cm² rectangular tissue culture flasks containing 175 ml of PM119 medium for seven days. The flasks were positioned with their narrowest “width” dimension against an LED light source. The culture flasks were masked with an opaque white plastic to provide a 21.1 cm² rectangular opening for irradiance to reach the cultures. Incident irradiance was programmed at a 16 h light:8 hour dark cycle with a linear ramp up of irradiance from 0 to 1200 uE over 4 hous, after which the irradiance was held at for six hours at 1200 uE, and then a linear ramp down in irradiance from 1200 to 0 uE over a 4 h period (increasing or decreasing in 15 min intervals). Deionized H₂O was added to the cultures daily to replace evaporative losses. The temperature of the cultures was regulated by a water bath set at 25° C. Samples (5 mls) were removed on daily for assessing total organic carbon (TOC) as described in Example 3. Sampling was done 30 minutes prior to the end of the light cycle.

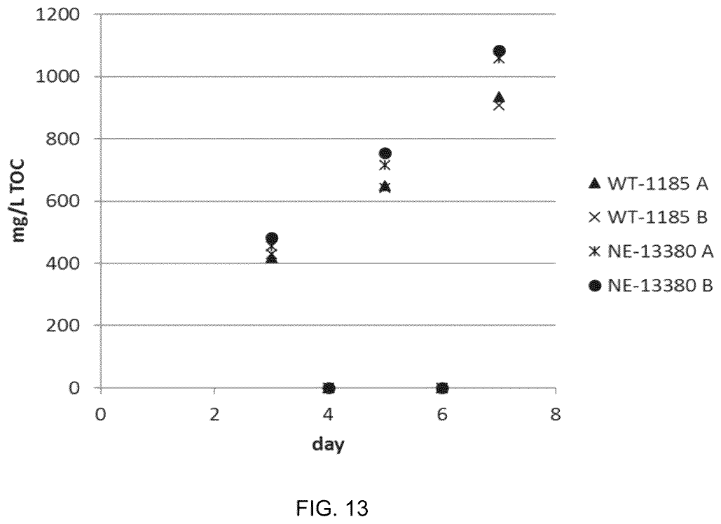

FIG. 13 shows that under these batch, nitrogen replete conditions the NE-13380 SGI1 mutant outperformed the wild type in biomass accumulation.

### Example 13

### TOC and Lipid Productivity of Batch Cultures Under Nitrogen Limitation

The SGI1 mutant was also assessed for biomass and lipid accumulation under nitrogen limitation. SGI1 knockout strain NE-7843 and the wild type progenitor strain WT-1185 were cultured in a batch productivity assay in nitrogen deplete medium, i.e., the culture medium had no source of nitrogen for cell growth. The production cultures were inoculated to an initial OD730 of 0.5 from seed (scale-up) cultures that were grown in PM074 medium that included 8.8 mM nitrate.

After inoculation, ZnCys knockout strain NE-7843 and wild type strain WT-1185 were grown in triplicate 175 ml cultures in a batch assay in 75 cm² rectangular tissue culture flasks for seven days.

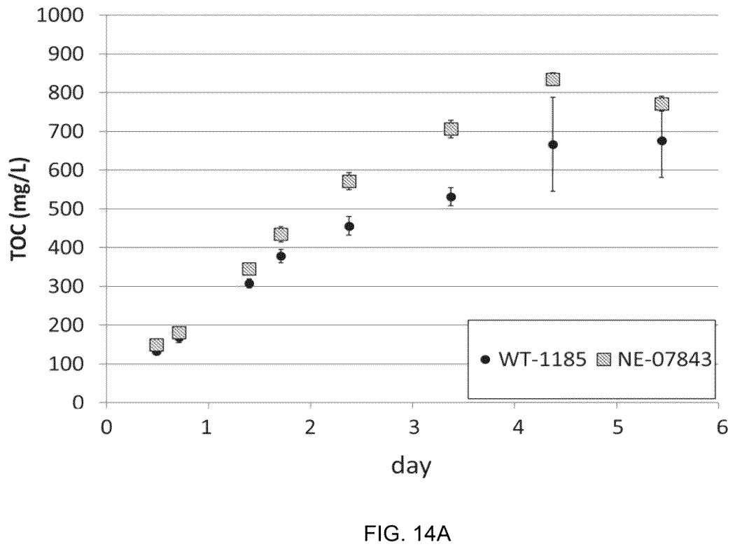

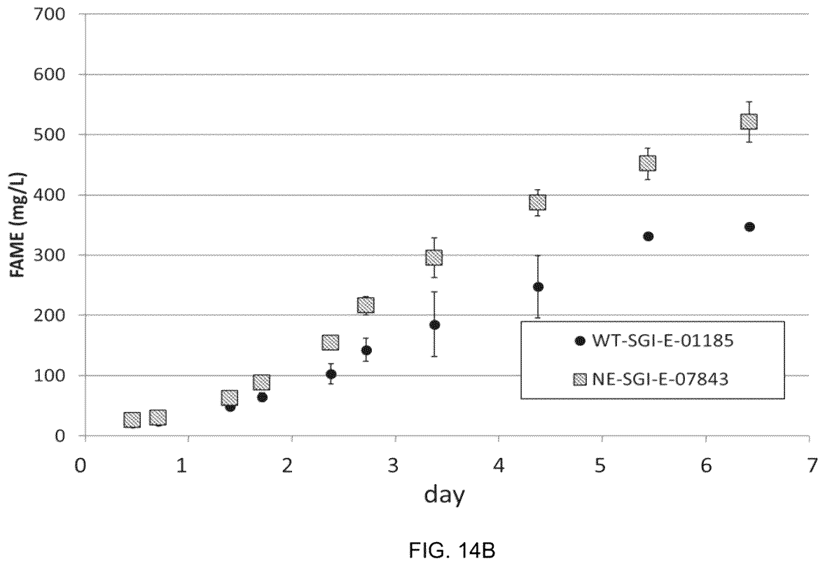

FIG. 14A shows the accumulation of volumetric TOC under N-replete batch conditions. Biological duplicate data are shown for WT-1185 and NE-13380, where the NE-13380 mutant outperformed the wild type strain in biomass accumulation. FIG. 14B shows the SGI1 mutants also outperforms the wild type strain in lipid (FAME) accumulation, accumulating approximately 50% more FAME than wild type over the course of the one week assay.

### Example 14

### High Irradiance Cultures

Growth rate improvements of the SGI1 mutants disclosed herein have been observed in multiple culture conditions (Table 8). Cultures were maintained at high dilution under constant high light by a continuous dilution system that introduced fresh nutrient replete culture medium and discharged an equal volume of culture. Parachlorella SGI1 mutants demonstrated an 18-20% increased growth rate in moderate to high light conditions (500-1,400 μE), and in extremely high light (5,000 μE) the SGI1 mutant was observed to grow at approximately 5-fold the rate of the wildtype progenitor strain (Table 8). The dramatic difference in growth rate under high light conditions can be used to select for SGI1 mutants.

TABLE 8
Growth Rates of Parachlorella Strains in Low Density, High Light
Conditions

| | Specific Daily Growth Rate | | | Rate of Division (h) | | |
|---|---|---|---|---|---|---|
| | 500 | 1,400 | 5,000 | 500 | 1,400 | 5,000 |
| Strain | μE^{a} | μE^{b} | μE^{c} | μE^{a} | μE^{b} | μE^{c} |
| WT-1185^{d} | 1.75 | 1.92 | 0.16 | 9.5 | 8.7 | 104.0 |
| NE-13380^{e} | 2.1 | 2.27 | 0.85 | 7.9 | 7.3 | 19.6 |
| Improvement^{f} | 20.0% | 18.2% | 431.3% | 20.0% | 18.2% | 431.3% |

^{a}Growth rate in shake flask with daily subcultures and 500 μE constant light.
^{b}Growth rate in continuous culture kept at low density (OD₇₂₀ = 0.1) with 1,400 μE in T225 photobioreactor (CL-SCPA).
^{c}Growth rate in continuous culture kept at low density (OD₇₂₀ = 0.1) with 5,000 μE in FMT150 photobioreactor.
^{d}Wildtype Parachlorella.
^{e}Parachlorella with SGI1 mutation.
^{f} Increase in growth rate in SGI1 mutant in comparison with wildtype parent strain.

A small number of NE-13380 SGI1 mutant cells were inoculated into a photobioreactor with 400×10⁶ wild type Parachlorella WT-1185 cells that were being grown in continuous culture in low density conditions (1×10⁶ cells per ml) with high light (1,400 μE). Over a period of 60 days the growth rates slowly increased, such that after 60 days the culture had reached a specific daily growth rate of 2.27. The culture was then plated on agar plates for single colonies. Individual clones were picked and analyzed for growth rate and presence of SGI1 mutation. Remarkably, all of the clones isolated from the plates contained the SGI1 mutation suggesting that it is possible to select for SGI1 type mutations through the use of low density, high light, continuous culture. For example, UV, chemical mutagenesis, random insertional mutagenesis, or other mutagenesis procedures can oprionally be performed, followed by low density, high light, continuous culturing for one, two, three, four, five, six, seven, eight, or more weeks to select for high productivity mutants such as SGI1-type mutants.

FIG. 7 shows a map of the SGI1 gene with positions of mutations found in various mutants marked. Strain NE-7843 was isolated in the screens disclosed in Examples 1-6, and strain NE-16980 was isolated using the screen disclosed in Example 7. Several additional mutants whose mutations are identified in FIG. 7 were isolated under the high light, low culture density selection of the present example.

### Example 15

### Identification and Targeting of the Tetraselmis SGI1 Gene

The genome of an environmental isolate of a wild type alga of a Tetraselmis species, referred to herein as Tetraselmis WT-105, was completely sequenced in-house (Synthetic Genomics, Inc., La Jolla, CA, USA). Analysis of the genome was most consistent with Tetraselmis sp. WT-105 being triploid. Based on reciprocal BLAST using the Parachlorella SGI polypeptide sequence as a query for translated sequences of the Tetraselmis WT-105 genome, three genes were identified: T-5172 (SEQ ID NO:85, having the protein-encoding sequence (or cDNA sequence) of SEQ ID NO:86), T-5185 (SEQ ID NO:87, having the protein-encoding sequence (or cDNA sequence) of SEQ ID NO:88), and T-5230 (SEQ ID NO:89, having the protein-encoding sequence (or cDNA sequence) of SEQ ID NO:90), considered to be alleles of the Tetraselmis WT-105 SGI1 gene. The proteins encoded by alleles T-5185 (SEQ ID NO:14) and T-5230 (SEQ ID NO:16) are highly homologous, demonstrating 98% amino acid sequence identity by BLAST. The protein encoded by the T-5172 allele (SEQ ID NO:15) is 90% identical to the protein encoded by the T-5230 allele (SEQ ID NO:16) and 92% identical to the protein encoded by the T-5185 allele (SEQ ID NO:14) by BLAST analysis. Each allele was determined to have a RR domain and a myb domain (see Table 3). As also provided in Table 3, the Tetraselmis SGI1 allelelic polypeptides T-5172, T-5185, and T-5230 were found to have scores of at least 400, namely, 403.0 (e-value 2e-118), 403.6 (e-value 9e-119), and 402.9 (e-value 3e-118), respectively, against an HMM developed for identifying SGI1 polypeptides (Example 6).

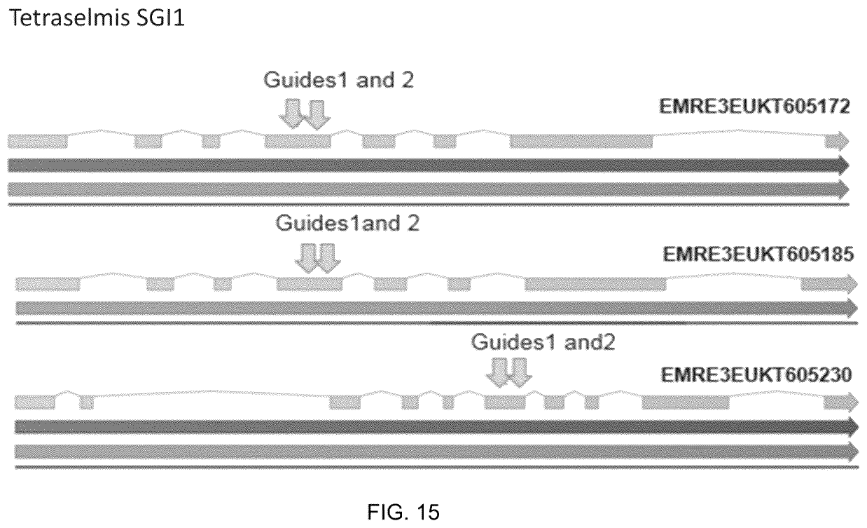

To generate a Tetraselmis SGI knockout strain, a Cas9 ribonucleoprotein complex (RNP) that included a CRISPR-Cas9 guide RNA (crRNA) and a CRISPR-Cas9 tracr RNA (trRNA) complexed with a Cas9 protein was introduced into Tetraselmis algal cells by particle bombardment. A selectable marker cassette to select transformants was introduced along with the Cas9 RNP on the gold microparticles. The tracr RNA used to make the RNPs for Tetraselmis transformation was a chemically-modified 67 nucleotide Alt-R® CRISPR-Cas9 tracrRNA purchased from IDT (Coralville, IA, USA). The crRNAs used in RNP transformations were chemically modified Alt-R® CRISPR-Cas9 crRNAs also purchased from IDT (Coralville, IA, USA) that included a 20 nucleotide target sequence (MA1, SEQ ID NO:91 or JC2, SEQ ID NO:92) present in the fourth exon of all three alleles of the SGI1 gene (see FIG. 15). The crRNAs also included a 16 nucleotide sequence complementary to a sequence of the tracrRNA.

Prior to forming the RNP complexes, the crRNAs and tracrRNA were preincubated to allow the crRNAs to hybridize with the tracrRNA. Each RNA was resuspended at a concentration of 100 μM and equal amounts of crRNA and tracrRNA RNA were combined in separate tubes in 20 μM aliquots, such that each RNA had a concentration of 50 μM in each annealing mixture, except that where two crRNAs were used in a single experiment, each crRNA had a concentration of 25 μM in the annealing mixture. The combined RNAs were heated to 95° C. for 5 minutes and then allowed to cool on the benchtop. (Annealed crispr and tracer RNAs that were not used immediately were stored at −20° C.)

A mixture of the annealed crispr and tracrRNAs (10 μl of the annealing mixture) with Cas9 protein was made in phosphate buffered saline (PBS), where the final concentration of Cas9 protein was 100 ug/mL and the final concentration of RNAs was 10 μM. The annealed crRNA(s) and trRNA and Cas9 protein were incubated 15 min. at room temperature to form RNPs.

To prepare the gold microparticles, protamine (100 μl of 1 mg/ml solution in PBS, made by heating to 55° C. to 65° C. and cooling to room temperature before use) was added to 5 mg gold particles (0.6 μm, BioRad), and the gold particles/protamine mixture was vortexed and then sonicated. A DNA fragment (SEQ ID NO:93) that included a selectable marker gene operably linked to the Tetraselmis RB40 promoter (SEQ ID NO:94) and Tetraselmis RB40 terminator (SEQ ID NO:95) was then added to the protamine/gold suspension, and the mixture was vortexed. The selectable marker DNA fragment was a PCR product that included a nourseothricin resistance gene codon optimized for Tetraselmis (SEQ ID NO:96) operably linked to the RB40 promoter and terminator. The five 5-most nucleotides of each primer (primer MCA1818, SEQ ID NO:97 and primer MCA1819, SEQ ID NO:98) used to produce the selectable marker cassette by PCR using a plasmid template included phosporothioate groups, such that the final selectable marker cassette fragment used in transformations was phosphorothioate-modified on each end.

The Cas9 RNP preparation (annealed RNAs/Cas9 protein) was added to the gold particles plus selectable marker DNA fragment in protamine solution immediately after addition of the selectable marker DNA fragment to the gold particles and the mixture was vortexed and then placed on ice for 2-4 hours. After the incubation on ice, the gold microparticle preparations were spun down briefly in a microfuge, the supernatant was removed, and 400 μL of 1.5 mg/ml PVP in PBS was added to each microparticle sample. The samples were vortexed, sonicated for 5-10 sec, and again vortexed, after which the PVP-suspended particles were pulled into a pre-dried segment of Tefzel™ tubing by attaching a syringe to flexible tubing that was connected to the Tefzel™ tubing, and drawing the particle mixture into the opposite end of the Tefzel™ tubing by application of suction via the syringe. The Tefzel™ tubing that included the gold particle preparation was set down for 5 min to allow the gold particles to settle, after which the PVP solution was gently pushed back out of the tubing via the syringe. The tubing was then rotated to distribute the gold particles over the inner circumference of the tubing, and allowed to air dry for approximately 5 minutes (during which time the tube was rotated one or more additional times to distribute the particles), after which the Tefzel™ tubing was attached of a nitrogen gas source and nitrogen gas was allowed to flow through the tubing at 0.1 L per min for one minute, and then at from about 0.3 to about 0.4 L per minute for 5-10 minutes to complete the drying. The Tefzel™ tubing coated on the interior surface with gold particles to which the selectable marker DNA and RNPs was adhered was then cut into 0.5 inch segments which were used as cartridges in a BioRad Helios® Gene Gun.

For microbombardment transformation, Tetraselmis strain WT-105 was cultured for approximately three days in a volume of 100 mls PM074 culture medium inocculated to an OD730 of 0.2. The starter culture was used to inocculate a 1 liter cutlure at 3×10⁵ cells/ml, and the one liter culture was allowed to grow out for another 3 days. Cells were resupsended to a concentration of 10⁸ cells per ml, with approximately 10⁷ cells (100 μl) plated within a 4 cm diameter circle on 2% agar PM074 medium plates for each bombardment. Plated cells were allowed to dry in the hood, and were then bombarded with the prepared cartridges from a distance of approximately 5 cm. using helium set at 600 psi. The following day the plates were flooded with PM147 medium and replated on PM147 plates that included 200 μg/ml nourseothricin.

After 10-14 days of growth on selective plates, dozens of colonies were repatched onto selective PM147 plates that included 200 μg/ml nourseothricin. Surviving patches were PCR screened for mutations in the SGI1 locus. It was found that the selectable marker gene cassette (SEQ ID NO:93) did not insert into the Cas9-disrupted locus of transformed Tetraselmis cells, therefore, the SGI1-targeted mutations were mostly small insertions or deletions (indels) at the SGI1 target site. PCR fragments generated from the clones were Sanger sequenced to detect these mutation events. Many of the transformants were found to have a mutation at the target site. Ten of these were chosen for cloning of genomic DNA in E. coli to sequence each allele. All of the clones in Table 8 were determined by sequencing to be triple knockouts (i.e., homozygous for the mutation) with the exception of clone 8, which was heterozygous for the mutant allele.

TABLE 9
Mutant Alleles in Isolated Tetraselmis Clones after RNP
Transformation Using SGI-targeting Guide RNAs

| Clone | | Guide(s) Used in | |
|---|---|---|---|
| Number | Strain ID | Transformation | Mutation Type |
| 1 | | MA1 | large insertion |
| 2 | STR24094 | MA1 + JC2 | 7 bp insertion |
| 3 | | MA1 + JC2 | large insertion |
| 4 | | MA1 + JC2 | 5 bp insertion |
| 5 | | MA1 + JC2 | 13 bp insertion |
| 6 | STR24095 | MA1 | 5 bp deletion |
| 7 | STR24096 | MA1 | 9 bp deletion |
| 8 | STR24097 | MA1 | 1 bp insertion |
| 9 | | MA1 + JC2 | large insertion |
| 10 | STR24098 | MA1 + JC2 | 15 bp insertion |

### Example 16

### Photophysiology of Targeted Tetraselmis SGI1 Mutants

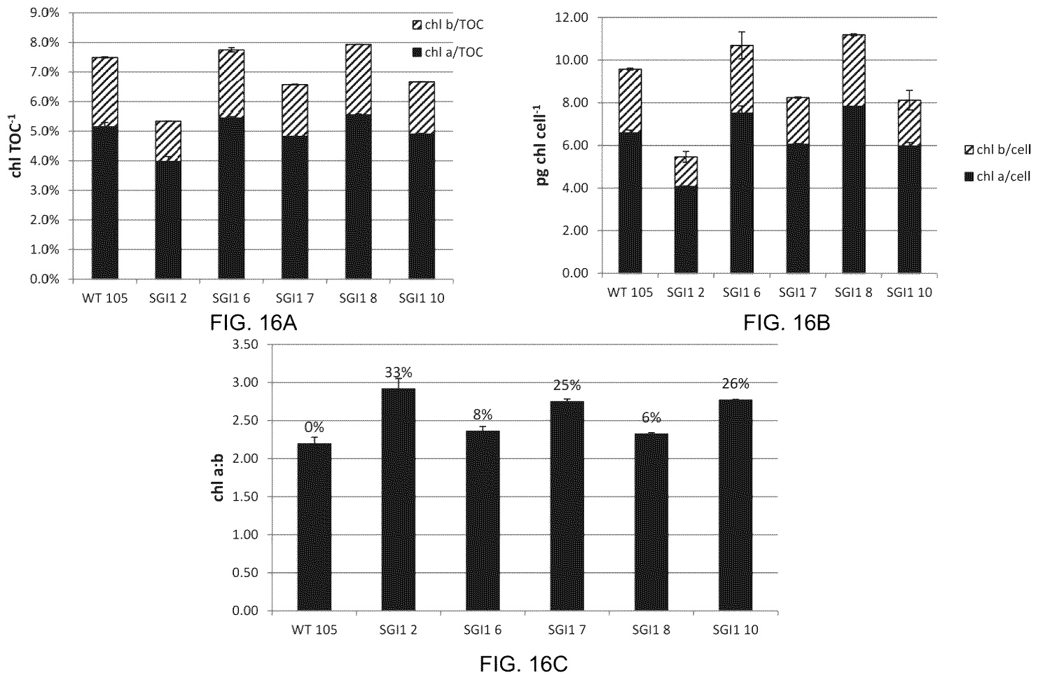

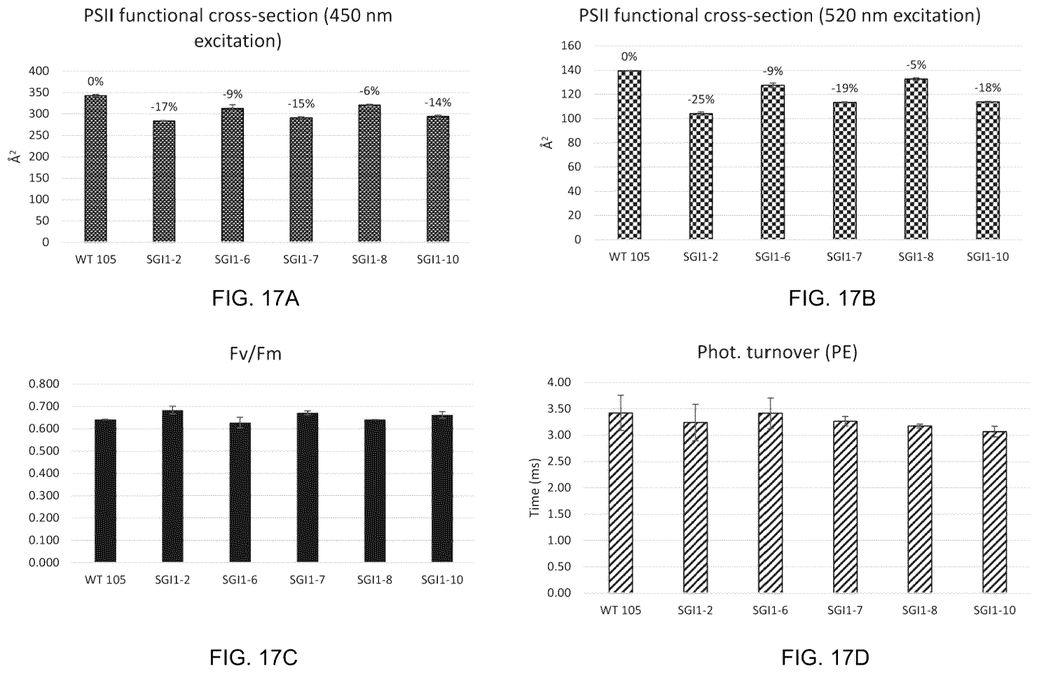

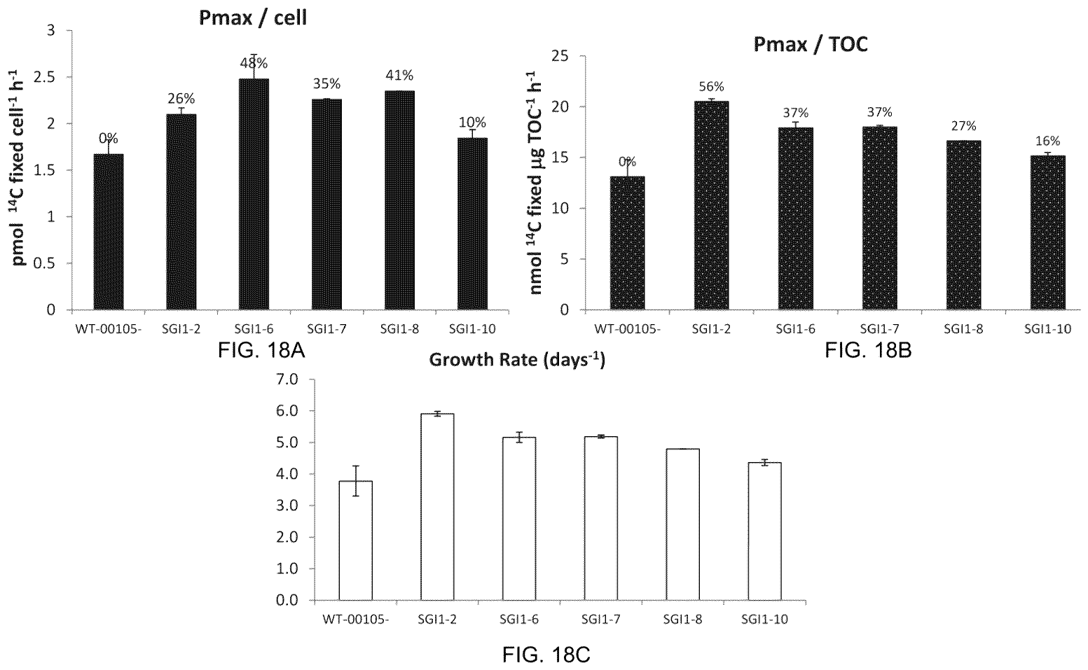

To characterize these clones, five of the ten clones listed in Table 8 (clones 2, 6, 7, 8, and 10) were cultured photoautotrophically under low, 24 hour/day light (140 mol photons m⁻² s⁻¹) in replete PM074 culture medium that did not include a reduced carbon source and included nitrate as the nitrogen source. The cultures were diluted daily to maintain a fixed cell number. Chlorophyll was extracted and FIRe and ¹⁴C Pₘₐₓ measurements were performed as described in Example 7. The results are shown in FIGS. 16A, 16B, and 16C; FIGS. 17A, 17B, 17C; and 17D, and FIGS. 18A, 18B, and 18C. In these low light conditions, the chorophyll a:b ratio was increased with respect to the wild type strain for all of the mutants (FIG. 16C), with clones 2, 7 and 10 demonstrating decreases in total chlrophyll and particularly in chlorophyll b on both a per TOC (FIG. 16A) and per cell (FIG. 16B) basis. All of the clones demonstrated a reduction in the PSII cross-sectional size measured at both 450 and 520 nm excitation, with clones 2, 7, and 10 showing the greatest reduction in PSII cross-sectional size with respect to wild type (FIGS. 17A and 17B). Clones 2, 7 and 10 also demonstrated the greatest increases in Fv/Fm (FIG. 17C), while all clones demonstrated reduced photosynthetic turnover times PSII (FIG. 17D). ¹⁴C Pₘₐₓ was increased for all clones on both a per cell and per TOC basis (FIGS. 18A and 18B), and all clones had higher growth rates than wild type (FIG. 18C).

Clones 2 and 7, given the strain names STR24094 and STR24096, respectively, were then tested in a semi-continuous assay that used a diel light program with a 14 hour light period that simulated the light intensity of a spring day in Southern California, peaking in intensity at approximately 2,000 mol photons m⁻² s⁻¹ at solar noon. Three biological replicates of each strain were cultured simultaneously and the results of the three cultures were averaged. The culture bottles were partially wrapped in foil to reflect light not initially absorbed back into the cultures. The culture medium was PM153, which included urea as the nitrogen source and did not include a reduced carbon source (i.e., the strains were cultured photoautotrophically), and cultures were diluted back by 60% daily. As summarized in Table 10, that under these conditions, SGI1 mutant strains STR24094 and STR24096 have increased Fv/Fm, reduced PSII antenna size (measured with 520 nm excitation), and increased PSII per TOC. Chlrophyll as a percentage of TOC is reduced in the Tetraselmis SGI1 mutants, and the ratio of chlorophyll a to chlorophyll b is increased. The photosynthetic rate, measured as ¹⁴C incorporation, is increased from about 15 to 20% in these mutants. Productivity, reported as grams TOC produced per meter per day, is increased approximately 6.5% in STR24094 and approximately 10% in STR24096.

TABLE 10
Photophysiology of Tetraselmis SGI1 Mutants in Semi-Continuous Diel Cultures

| | | | | | | | ¹⁴C Pₘₐₓ | |
|---|---|---|---|---|---|---|---|---|
| | FIRe | | | | Chlorophyll | | nmol | |
| | | | τ'Qa | | | Chl | ¹⁴C/ug | Productivity |
| Strain | Fv/Fm | PSII | (ms) | PSII/TOC | Chl/TOC | a:b | TOC/h | gm⁻²day⁻¹ |
| WT-105 | 0.621 | 148 | 5.0 | 17 | 7.8% | 2.0 | 25.6 | 15.5 |
| STR24094 | 0.683 + | 108 − | 3.6 − | 20 + | 6.4% − | 2.8 + | 30.8 + | 16.5 + |
| | 10% | 27% | 28% | 17.64% | 17.9% | 40% | 20.3% | 6.45% |
| STR24096 | 0.677 + | 117 − | 4.3 − | 21 + | 7.6% − | 2.5 + | 29.7 + | 17.1 + |
| | 9.0% | 21% | 14% | 23.5% | 2.5% | 25% | 16% | 10.3% |

### Example 17

### Productivity of Tetraselmis SGI1 Mutants in a Constant Light Semi-Continuous Culture System

In the CL-SCUBA (Constant Light Semi-Continuous Urea-Based Assay), photoautotrophic cultures of the Tetraselmis SGI1 mutants were grown over several days in constant light semi-continuous mode with culture samples removed at the same time each day for biomass determination. The light was programmed at 2000 μmol photons m⁻² sec⁻¹. In this assay 420 ml of PM153 culture medium (that included urea as a nitrogen source) in a 500 ml square flask was inoculated with seed culture of a given strain. Three cultures were initiated per strain. The flasks included stir bars and had stoppers having tubing connected with syringe filters for delivering CO₂ enriched air (1% CO₂) that was bubbled through the cultures. The flasks were aligned with an aperture of 0.0875 m² toward the light and “depth” dimension of the flasks, extending back from the light source, was 8 cm. The cultures were diluted daily by removing 40% of the culture volume and replacing it with fresh PM153 culture medium diluted to adjust for the increase in salinity due to evaporation occurring in the cultures. Samples for TOC analysis were taken from the culture removed for the dilution after the cultures had reached steady-state.

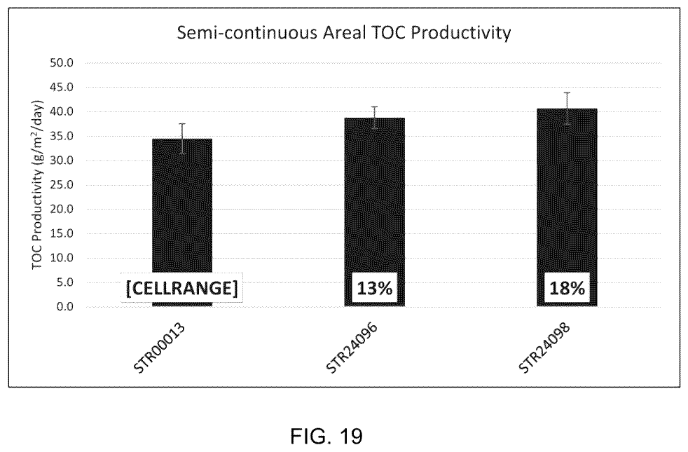

FIG. 19 shows that under these constant light semi-continuous culture conditions, Tetraselmis SGI1 mutants STR24096 and STR24096 demonstrated 13% and 18% increased biomass, respectively, with respect to wild type cells (strain STR00013).

### Example 18

### Productivity of Tetraselmis and Parachlorella SGI1 Mutants in a Diel Semi-Continuous Culture System

In the diel SCUBA (Semi-Continuous Urea Based Assay), photoautotrophic cultures of the mutants were grown over several days in diel light semi-continuous mode. The light was programmed to mimic an average spring day in the Imperial Valley of California ranging from darkness to 2000 μmol photons m⁻² sec⁻¹ at noon. Samples were taken at “dusk” each day. In this assay 420 ml of urea based PM153 culture medium in a 500 ml square flask was inoculated with seed culture of a given mutant strain. Three cultures were initiated per strain. The flasks included stir bars and had stoppers having tubing connected with syringe filters for delivering CO₂ enriched air (1% CO₂) that was bubbled through the cultures. The flasks were aligned with a aperture towards the light of 0.0875 m² and “depth” dimension of the flasks, extending back from the light source, was 8 cm. The cultures were diluted daily by removing 40% of the culture volume and replacing it with fresh PM153 culture medium diluted to adjust for the increase in salinity due to evaporation occurring in the cultures. Culture samples removed daily for biomass determination after the cultures had reached steady state. Samples for TOC analysis were taken from the culture removed for the dilution.

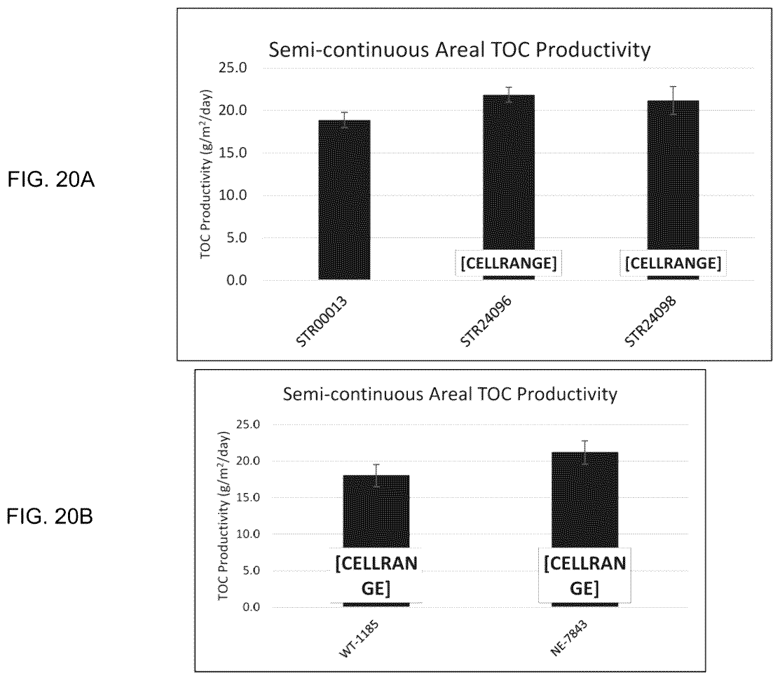

FIG. 20A shows that under these conditions, Tetraselmis SGI1 mutants demonstrated between 12% and 16% increased biomass with respect to wild type cells.

In a parallel diel SCUBA assay performed according to the same protocol, the classically-derived Parachlorella SGI1mutant NE-7843 was tested in triplicate cultures alongside triplicate cultures of the Parachlorella wild type strain WT-1185. FIG. 20B shows that under this culutre regime, Parachlorella SGI1 mutant NE-7843 demonstrates an 18% increase in productivity with respect to the wild type strain.

### Example 19

### Analysis of Arabidopsis SGI1 Mutant

To determine the effects of an SGI1 mutation in higher plants we first identified the closest relatives of the Parachlorella SGI1 gene in Arabidopsis thaliana. Using the bioinformatic analysis disclosed in Example 6, the closest A. thaliana homolog to the Parachlorella SGI1 was determined to be ARR2 (SEQ ID NO:22).

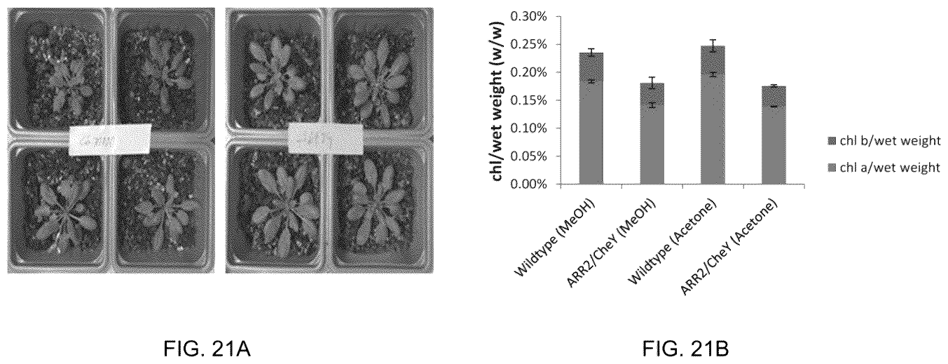

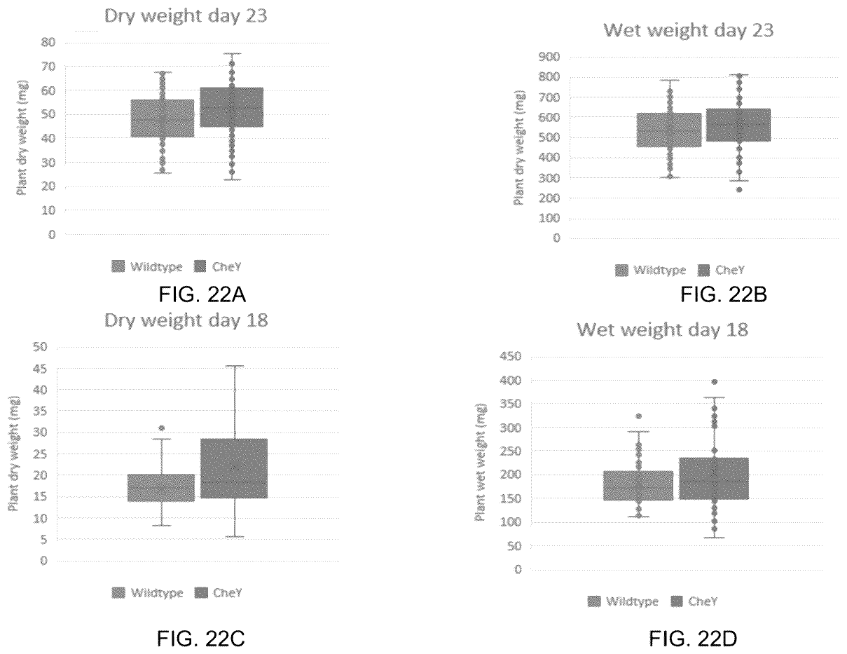

Seeds from A. thaliana plants with mutations in ARR1 and ARR2 as well as a wildype control were obtained from the Arabidopsis resource center (ABRC). Seeds were planted in soil (50% MiracleGro fertilized seed starting potting mix, 25% perlite, 25% vermiculite). The planted seeds were vernalized at 4° C. for five days prior to transfer to a conviron for growth. The plants were grown side by side on four different occasions in a conviron set at 25° C. The plants received ˜70 μE of light with a 12 hour dark period. Growth of the ARR1 mutant was substantially slower than both the wildtype and the ARR2 plants and we therefore focused on comparisons between the wildtype and ARR2/SGI1mutant plants (hereafter referred to as SGI1 plants). During the first two growth experiments, we observed a visual improvement in growth of the SGI1 mutant plants (FIG. 21A). Parachlorella SGI1 mutants have reduced chlorophyll and we therefore determined the chlorophyll content in Arabidopsis wildtype and SGI1 leaves. Chlorophyll was extracted using with both acetone and methanol from three wildtype and three ARR2 mutant plants (two leaves from each plant). We observed a significant reduction in the amount of chlorophyll in the SGI1 mutant plants (FIG. 21B). In the last two growth experiments we harvested plants at day 23 and day 18 to determine wet weights and dry weights of the plant shoots. Although we observed a high variation between individual plants and a high overlap between wildtype and SGI1 plants, the SGI1 plants consistently had a higher average shoot weight of about 10% at 23 days (FIGS. 22A and 22B) and about 40% at 18 days (FIGS. 22C and 22D). As found in the green algae (Parachlorella and Tetraselmis) with SGI1 mutations, we observed reduced chlorophyll and an increase in average biomass during vegetative growth in an A. thaliana SGI1 mutant, indicating that SGI mutations can be associated with increased productivity in other higher plants as well.

Although the invention has been described with reference to the above examples, it will be understood that modifications and variations are encompassed within the spirit and scope of the invention. Accordingly, the invention is limited only by the following claims.

What is claimed is:

1. A mutant Chlorophyte algal organism having an attenuated gene encoding an SGI1 polypeptide having at least 90% sequence identity to a SEQ ID NO: 17; and wherein the photosynthetic organism has at least 25% higher lipid productivity than a control organism grown or cultured under the substantially the same conditions.

2. The mutant Chlorophyte algal organism of claim 1 wherein the attenuation is a deletion or a disruption in the gene encoding the SGI1 polypeptide.

3. The mutant Chlorophyte algal organism of claim 2 wherein the gene encoding the SGI1 polypeptide has at least 95% sequence identity to a SEQ ID NO: 17.

4. The mutant Chlorophyte algal organism of claim 1 wherein the algal organism has at least 40% higher lipid productivity than the control organism.

5. The mutant photosynthetic organism according to claim 1, wherein the mutant photosynthetic organism has reduced chlorophyll with respect to the control photosynthetic microorganism.

6. The mutant photosynthetic organism according to claim 4, wherein the mutant photosynthetic organism has an increased chlorophyll a:b ratio with respect to the control photosynthetic organism.

7. The mutant photosynthetic organism according to claim 1, wherein the mutant photosynthetic organism has a reduced photosystem II (PSII) antenna size with respect to the control photosynthetic organism.

8. The mutant photosynthetic organism according to claim 1, wherein the mutant photosynthetic organism has a higher 14C Pmax with respect to the control photosynthetic organism.

9. The mutant photosynthetic organism according to claim 1, wherein the mutant photosynthetic organism has a higher Fv/Fm than the control organism.

10. The mutant photosynthetic organism according to claim 1, wherein the mutant photosynthetic organism has a higher protein content than the control organism.

11. The mutant algal microorganism according to claim 1, wherein the mutant algal microorganism is a Chlorophyte of the Class Trebouxiophyceae.

12. The mutant algal microorganism according to claim 11, wherein the mutant algal microorganism is a species of the genus Oocystis.

13. The mutant algal microorganism according to claim 12, wherein the cross-sectional unit size of the PSII antenna size is reduced by at least 25%, with respect to the cross-sectional unit size of the PSII antenna of the control microorganism.

14. The mutant algal microorganism according to claim 12, wherein the Pmax (14C) is increased by at least 25% with respect to the Pmax (14C) of the control microorganism.

15. The mutant algal microorganism according to claim 1, wherein the mutant algal microorganism has at least 20% less chlorophyll with respect to the control algal microorganism.

16. The mutant algal microorganism according to claim 1, wherein the mutant algal microorganism has at least 20% less chlorophyll b with respect to the control algal microorganism.

17. The mutant algal microorganism according to claim 16, wherein the mutant algal microorganism has at least 20% more protein as a percent of total organic carbon than the control algal microorganism.

18. The mutant algal microorganism according to claim 1, wherein the mutant algal microorganism has reduced expression of at least 10 LHC genes with respect to the control algal microorganism.

19. A method of making an lipid product comprising culturing the algal mutant of claim 1 to produce the lipid product.

20. The method according to claim 19, further comprising recovering the lipid product from the culture.
